(19) United States
(12) Patent Application Publication
(10) Pub. No.: US 2026/0258408 A1
(43) Pub. Date: Sep. 3, 2026

(54) MAPT SIRNA AND USES THEREOF

(71) Applicant: Janssen Pharmaceutica NV, Beerse (BE)

(72) Inventors: Lujia Zhou, Beerse (BE); Diederik Moechars, Beerse (BE); Jin Li, Brisbane, CA (US); Marija Prhavc, Brisbane, CA (US); Sijin Guo, Brisbane, CA (US); Mahesh Ramaseshan, Brisbane, CA (US)

(21) Appl. No.: 18/846,684

(22) PCT Filed: Mar. 16, 2023
(86) PCT No.: PCT/EP2023/056783

§ 371 Date: Sep. 13, 2024

Related U.S. Application Data
(60) Provisional application No. 63/320,687, filed on Mar. 16, 2022.
(60) Provisional application No. 63/320,684, filed on Mar. 16, 2022.

(51) Int. Cl. C12N 15/113

(52) U.S. Cl. CPC C12N 15/113; C12N 2310/14; C12N 2310/315; C12N 2310/317; C12N 2310/321; C12N 2310/322; C12N 2310/346; C12N 2310/3515

(57) ABSTRACT

Provided herein are small interfering ribonucleic acid (siRNA) agents targeting a microtubule-associated protein tau (MAPT) gene and compositions comprising such siRNA agents.

### 1. CROSS-REFERENCE TO RELATED APPLICATIONS

This application claims the benefit of U.S. Provisional Application No. 63/320,684, filed on Mar. 16, 2022, and U.S. Provisional Application No. 63/320,687, filed on Mar. 16, 2022, each of which is incorporated herein by reference in its entirety.

### 2. INTRODUCTION

Provided herein are small interfering ribonucleic acid (siRNA) agents targeting a microtubule-associated protein tau (MAPT) gene and compositions comprising such siRNA agents.

### 3. BACKGROUND

Tau is normally a soluble protein associated with microtubules in neurons with its most well-characterized function being microtubule polymerization under physiological conditions. Under pathological conditions, however, Tau becomes hyperphosphorylated and dissociated from the microtubules, forms intracellular soluble oligomer species and insoluble aggregates rich in 3-sheet structure. Certain Tau assemblies can be released and transmitted to neurons in anatomically connected regions to induce or “seed” the native monomeric Tau to oligomerize into aggregates, thus propagating Tau aggregates in a prion-like manner. The molecular mechanism whereby Tau exerts its pathogenic action in Alzheimer's disease is not known today. What is however clear is that the pathogenic action of Tau involves toxic gain-of-functions exerted by small or large aggregated forms Tau protein species.

There is a need for compounds and agents that inhibit the expression of the MAPT gene, for example, to study Alzheimer's disease.

### 4. SUMMARY

In one aspect, provided herein are non-naturally occurring small interfering ribonucleic acid (siRNA) agents comprising a sense strand and an antisense strand forming a double stranded region.

In some embodiments, provided herein is a non-naturally occurring small interfering ribonucleic acid (siRNA) agent comprising a sense strand and an antisense strand forming a double stranded region, wherein: (a) the antisense strand comprises or consists of a nucleotide sequence corresponding to any one of the antisense strand nucleotide sequences in Table 1; (b) the sense strand comprises or consists of a nucleotide sequence corresponding to any one of the sense strand nucleotide sequences in Table 1; or (c) the antisense strand comprises or consists of a nucleotide sequence corresponding to an antisense nucleotide sequence in a row of Table 1, and the sense strand comprises or consists of a nucleotide sequence corresponding to the sense strand nucleotide sequence in the same row of Table 1.

In some embodiments, the antisense strand, the sense strand, or both of a siRNA agent described herein comprise at least one modified nucleotide. In some embodiments, the antisense strand and the sense strand of a siRNA agent described herein each comprises between three to twenty-three modified nucleotides. In some embodiments, the antisense strand of a siRNA agent described herein comprises all modified nucleotides. In some embodiments, the sense strand of a siRNA agent described herein comprises all modified nucleotides. In some embodiments, the antisense strand and sense strand of a siRNA agent described herein each comprise all modified nucleotides. In specific embodiments, the modified nucleotides are selected from the group consisting of a 2′O-methyl modified nucleotide, a deoxy-nucleotide, a 2′-fluoro modified nucleotide, a 2′-O-methyl-uridine, an inverted abasic nucleotide, a nucleotide comprising S-glycol nucleic acid (GNA), an unlocked nucleotide, a 5′-vinylphosphonate-2′-O-methyl-uridine, and combinations thereof.

In some embodiments, the antisense strand, the sense strand, or both of a siRNA agent described herein comprise at least one modified internucleoside linkage. In some embodiments, the antisense strand and the sense strand of a siRNA agent described herein each comprise a modified internucleoside linkage. In some embodiments, the antisense strand and the sense strand of a siRNA agent described herein each comprise two to five modified internucleoside linkages. In some embodiments, the modified internucleoside linkage is phosphorothioate.

In some embodiments, provided herein is a small interfering ribonucleic acid (siRNA) agent comprising a sense strand and an antisense strand forming a double stranded region, wherein: (a) the antisense strand comprises a nucleotide sequence of any one of the antisense strand nucleotide sequences in Table 2; (b) the sense strand comprises a nucleotide sequence of any one of the sense strand nucleotide sequences in Table 2; or (c) the antisense strand comprises a nucleotide sequence of an antisense strand nucleotide sequence in a row in Table 2, and the sense strand comprises a nucleotide sequence of the sense strand nucleotide sequence in the same row.

In some embodiments, provided herein is a small interfering ribonucleic acid (siRNA) agent comprising a sense strand and an antisense strand forming a double stranded region, wherein: (a) the antisense strand comprises a nucleotide sequence of any one of the antisense strand nucleotide sequences in Table 3; (b) the sense strand comprises a nucleotide sequence of any one of the sense strand nucleotide sequences in Table 3; or (c) the antisense strand comprises an antisense strand nucleotide sequence in a row in Table 3, and wherein the sense strand comprises the sense strand nucleotide sequence in the same row of Table 3.

In some embodiments, the antisense strand, the sense strand, or both the antisense and sense strand is conjugated to one or more lipophilic moieties. In some embodiments, the one or more lipophilic moieties is conjugated to one or more terminal positions of the siRNA agent, or internal positions of the double stranded region of the siRNA agent. In some embodiments, the one or more lipophilic moieties is conjugated via a linker or carrier. In some embodiments, the one or more lipophilic moieties is selected from the group consisting of cholesterol, L1, L2, L3, L4 (also referred to herein as “J2-CONC16U”), L5, L6, L7, L8, L9, L10, L11, L12, L13, L14, L15, L16, L17, L18, L19, L20, L21 (also referred to herein as “J2-CONCi6A”), L22 (also referred to herein as “J2-CONCi6C”), L23 (also referred to herein as “J2-CONCi6G”), and/or L24.

In some embodiments, provided herein is a small interfering ribonucleic acid (siRNA) agent comprising a sense strand and an antisense strand forming a double stranded region, wherein: (a) the antisense strand comprises a nucleotide sequence of any one of the antisense nucleotide sequences in Table 4; (b) the sense strand comprises a nucleotide sequence of any one the sense strand nucleotide sequences in Table 4, wherein the sense strand is conjugated to cholesterol or lipid moiety as shown in Table 4 for the sense strand nucleotide sequence; or (c) the antisense strand comprises an antisense strand nucleotide sequence in a row in Table 4, and the sense strand comprises the sense strand in the same row in Table 4, wherein the sense strand is conjugated to cholesterol or lipid as shown in Table 4 for the sense strand nucleotide sequence.

In some embodiments, provided herein is a small interfering ribonucleic acid (siRNA) agent comprising a sense strand and an antisense strand forming a double stranded region, wherein: (a) the antisense strand comprises a nucleotide sequence of any one of the antisense strand nucleotide sequences in Table 5; (b) the sense strand comprises a nucleotide sequence of any one of the sense strand nucleotide sequences in Table 5, wherein the sense strand is conjugated to cholesterol or lipid moiety as shown in Table 5 for the sense strand nucleotide sequence; or (c) the antisense strand comprises a nucleotide sequence of an antisense strand nucleotide sequence in a row in Table 5, and the sense strand comprises a nucleotide sequence of the sense strand nucleotide sequence in the same row of Table 5, and wherein the sense strand is conjugated to cholesterol or lipid moiety as shown in Table 5 for the sense strand.

In some embodiments, each strand of the siRNA agent is no more than 30 nucleotides in length. In some embodiments, each strand of the siRNA agent is 18, 19, 20, 21, 22, 23, 24, 25, 26, 27, 28 or 29 nucleotides in length.

In some embodiments, at least one strand of the siRNA agent comprises a 3′ overhang of at least 1 nucleotide. In some embodiments, at least one strand of the siRNA agent comprises a 3′ overhang of at least 2 nucleotides. In some embodiments, at least one strand of the siRNA agent comprises a 3′ overhang of at least 3 nucleotides. In some embodiments, both strands of the siRNA agent comprise a 3′ overhang of at least 1 or at least 2 nucleotides.

In some embodiments, the double stranded region of the siRNA agent is 15 to 19 nucleotides in length. In some embodiments, the double stranded region of the siRNA agent is 20 to 25 nucleotides in length. In some embodiments, the double stranded region of the siRNA agent is 15 to 25 nucleotides in length. In some embodiments, the double stranded region is 16, 17, 18, 19, 20, 21, 22, 23 or 24 nucleotides in length.

In some embodiments, a siRNA agent described herein comprises a targeting ligand.

In another aspect, provided herein is a cell containing the siRNA agent described herein. In a specific embodiment, the cell is in vitro or ex vivo.

In another aspect, provided herein is a composition comprising the siRNA agent described herein, and a carrier.

In one aspect, provided herein is an siRNA comprising a sense strand and an antisense strand forming a double stranded duplex, wherein
(a) the sense strand comprises 21 nucleotides having the sequence: UGCAAAUAGUCUACAAACCAA, wherein from the 5′end
   (i) the nucleotide at one or more positions selected from 1-6, 8 and 12-21 is optionally modified with 2′-O-methyl;
   (ii) the nucleotide at position 6 or 7 optionally comprises a lipophilic moiety;
   (iii) the nucleotide at one or more positions selected from 7 and 9-11 is optionally modified with 2′-deoxy-2′fluoro;
   wherein the sense strand optionally further comprises an inverse abasic nucleotide (InvAb) at the 5′ end linked to the nucleotide at position 1 and/or an inverse abasic nucleotide (InvAb) at the 3′ end linked to the nucleotide at position 21, and
   wherein the sense strand optionally comprises at least two phosphorothioate linkages at the 5′end and/or at least two phosphorothioate linkages at the 3′end; and
(b) the antisense strand comprises 21 nucleotides having the sequence: UUGGNNNGUAGACUAUUUGCA, wherein from the 5′ end
   (i) the nucleotide N at position 5 is U or T; the nucleotide N at position 6 is U or T; and the nucleotide N at position 7 is U or T;
   (ii) the nucleotide at position 1 is optionally modified with vinyl phosphonate 2′-O-methyl or cis-cyclobutyl phosphonate 2′-O-methyl;
   (iii) the nucleotide at one or more positions selected from 2, 6, 8, 9, 14, and 16 is optionally modified with 2′-deoxy-2′fluoro;
   (iv) the nucleotide at one or more positions selected from 1, 3-13, 15, and 17-21 is optionally modified with 2′-O-methyl;
   (v) optionally, the nucleotide at one or more positions selected from 4-7 is a deoxynucleotide;
   (vi) optionally, the nucleotide at position 7 is a 3′-O-methyl modified nucleotide with 2′-5′ linked phosphate, a (L)-α-threofuranosyl modified nucleotide, or a glycol nucleic acid (GNA), or an unlocked nucleic acid (UNA);
   wherein the antisense strand optionally further comprises two nucleotides CA linked to the nucleotide at position 21 such that the antisense strand comprises 23 nucleotides having the sequence UUGGNNNGUAGACUAUUUGCACA, wherein the nucleotide C at position 22 and/or the nucleotide A at position 23 is optionally modified with 2′-O-methyl, and
   wherein the antisense strand optionally comprises at least two phosphorothioate linkages at the 5′end and/or at least two phosphorothioate linkages at the 3′end; and
   wherein the base pair at position 1 of the 5′-end of the antisense strand of the duplex is an AU base pair.

In some embodiments, in the sense strand, the nucleotide at two or more positions selected from 1-6, 8 and 12-21 is modified with 2′-O-methyl. In some embodiments, in the sense strand, the nucleotide at three or more positions selected from 1-6, 8 and 12-21 is modified with 2′-O-methyl. In some embodiments, in the sense strand, the nucleotide at five or more positions selected from 1-6, 8 and 12-21 is modified with 2′-O-methyl. In some other embodiments, in the sense strand, the nucleotide at each of positions 1-5, 8, and 12-20 is modified with 2′-O-methyl. In some embodiments, in the sense strand, the nucleotide at two or more positions selected from 7 and 9-11 is modified with 2′-deoxy-2′fluoro. In some embodiments, in the sense strand, the nucleotide at each of positions 9-11 is modified with 2′-deoxy-2′fluoro. In yet some other embodiments, in the sense strand, the nucleotide at position 6 is modified with 2′-O-methyl and the nucleotide at position 7 comprises a lipophilic moiety. In some embodiments, the lipophilic moiety is L4. In some embodiments, in the sense strand, the nucleotide at position 6 comprises a lipophilic moiety and the nucleotide at position 7 is modified with 2′-deoxy-2′fluoro. In some embodiments, the lipophilic moiety is L21. In some embodiments, the sense strand further comprises an InvAb at the 5′ end linked to the nucleotide at position 1. In some other embodiments, the sense strand comprises two phosphorothioate linkages at both the 5′end and the 3′end.

In some embodiments, in the antisense strand, the nucleotide at two or more positions selected from 1, 3-13, 15, and 17-21 is modified with 2′-O-methyl. In some embodiments, in the antisense strand, the nucleotide at three or more positions selected from 1, 3-13, 15, and 17-21 is modified with 2′-O-methyl. In some embodiments, in the antisense strand, the nucleotide at five or more positions selected from 1, 3-13, 15, and 17-21 is modified with 2′-O-methyl. In some embodiments, in the antisense strand, the nucleotide at each of positions 1, 3, 10-13, 15, and 17-21 is modified with 2′-O-methyl. In some other embodiments, in the antisense strand, the nucleotide at two or more positions selected from 2, 6, 8, 9, 14, and 16 is modified with 2′-deoxy-2′fluoro. In some embodiments, in the antisense strand, the nucleotide at each of positions 2, 14 and 16 is modified with 2′-deoxy-2′fluoro. In some other embodiments, in the antisense strand, the nucleotides at position 4 and at position 5 are each modified with 2′-O-methyl. In some embodiments, in the antisense strand, the nucleotide at position 4 is modified with 2′-O-methyl and the nucleotide at position 5 is a deoxynucleotide. In some embodiments, in the antisense strand, the nucleotide at position 4 is a deoxynucleotide and the nucleotide at position 5 is modified with 2′-O-methyl. In some embodiments, in the antisense strand, the nucleotides at positions 4, 5, and 7 are each modified with 2′-O-methyl, and the nucleotide at position 6 is modified with 2′-deoxy-2′fluoro. In some embodiments, in the antisense strand, the nucleotides at positions 8 and 9 are either both modified with 2′-O-methyl, are both modified with 2′-deoxy-2′fluoro. In some embodiments, the antisense strand comprises two phosphorothioate linkages at both the 5′end and the 3′end.

In some embodiments, the sense strand comprises nucleotides having the sequence of SEQ ID NO: 335, 339, 341, 507, 509, 511, 513, 515, 521, 527, 531, 533, or 567. In some embodiments, the antisense strand comprises nucleotides having the sequence of SEQ ID NO: 336, 340, 342, 508, 510, 512, 514, 516, 522, 528, 532, 534, or 568.

In another aspect, provided herein is an siRNA comprising a sense strand and an antisense strand forming a double stranded duplex, wherein the sense strand comprises 21 nucleotides having the sequence: CAAGUCCAAGAUCGGCUCCAA, wherein from the 5′end
(i) the nucleotide at one or more positions selected from 1-6, 8, and 12-21 is optionally modified with 2′-O-methyl;
(ii) the nucleotide at one or more positions selected from 9-11 is optionally modified with 2′-deoxy-2′fluoro;
(iii) the nucleotide at position 7 optionally comprises a lipophilic moiety; wherein the sense strand optionally further comprises an inverse abasic nucleotide (InvAb) at the 5′ end linked to the nucleotide at position 1 and/or an inverse abasic nucleotide (InvAb) at the 3′ end linked to the nucleotide at position 21, and wherein the sense strand optionally comprises at least two phosphorothioate linkages at the 5′end and/or at least two phosphorothioate linkages at the 3′end; and
(a) the antisense strand comprises 21 nucleotides having the sequence: UUGGAGCCGAUCUUGGACUUG, wherein from the 5′end
   (i) the nucleotide at position 1 is optionally modified with vinyl phosphonate 2′-O-methyl or cis-cyclobutyl phosphonate 2′-O-methyl;
   (ii) the nucleotide at one or more positions selected from 2, 14, and 16 is optionally modified with 2′-deoxy-2′fluoro;
   (iii) the nucleotide at one or more positions selected from 1, 3-6, 8-13, 15, and 17-21 is optionally modified with 2′-O-methyl;
   (iv) optionally, the nucleotide at one or more positions selected from 4, 5, and 7 is a deoxynucleotide;
   (v) optionally, the nucleotide at position 7 is s a 3′-O-methyl modified nucleotide with 2′-5′ linked phosphate, a (L)-α-threofuranosyl (3′-2′) modified nucleotide, or a glycol nucleic acid (GNA), or an unlocked nucleic acid (UNA);
   wherein the antisense strand optionally comprises at least two phosphorothioate linkages at the 5′end and/or at least two phosphorothioate linkages at the 3′end; and
wherein the base pair at position 1 of the 5′-end of the antisense strand of the duplex is an AU base pair.

In some embodiments, in the sense strand, the nucleotide at two or more positions selected from 1-6, 8 and 12-21 is modified with 2′-O-methyl. In some other embodiments, in the sense strand, the nucleotide at three or more positions selected from 1-6, 8 and 12-21 is modified with 2′-O-methyl. In some embodiments, in the sense strand, the nucleotide at five or more positions selected from 1-6, 8 and 12-21 is modified with 2′-O-methyl. In some other embodiments, in the sense strand, the nucleotide at each of positions 1-6, 8, and 12-21 is modified with 2′-O-methyl. In some embodiments, in the sense strand, the nucleotide at two or more positions selected from 9-11 is modified with 2′-deoxy-2′fluoro. In some other embodiments, in the sense strand, the nucleotide at each of positions 9-11 is modified with 2′-deoxy-2′fluoro. In some embodiments, in the sense strand, the nucleotide at position 7 comprises lipophilic moiety L22. In some embodiments, the sense strand further comprises an InvAb at both the 5′ end linked to the nucleotide at position 1 and at the 3′ end linked to the nucleotide at position 21. In some embodiments, the sense strand comprises two phosphorothioate linkages at both the 5′end and the 3′end.

In some embodiments, in the antisense strand, the nucleotide at two or more positions selected from 1, 3-6, 8-13, 15, and 17-21 is modified with 2′-O-methyl. In some embodiments, in the antisense strand, the nucleotide at three or more positions selected from 1, 3-6, 8-13, 15, and 17-21 is modified with 2′-O-methyl. In some embodiments, in the antisense strand, the nucleotide at five or more positions selected from 1, 3-6, 8-13, 15, and 17-21 is modified with 2′-O-methyl. In some embodiments, in the antisense strand, the nucleotide at each of positions 1, 3, 6, 8-13, 15, and 17-21 is modified with 2′-O-methyl. In some embodiments, in the antisense strand, the nucleotide at two or more positions selected from 2, 14, and 16 is modified with 2′-deoxy-2′fluoro. In some embodiments, in the antisense strand, wherein in the antisense strand, the nucleotide at each of positions 2, 14, and 16 is modified with 2′-deoxy-2′fluoro. In some embodiments, in the antisense strand, the nucleotides at position 4 and at position 5 are each modified with 2′-O-methyl. In some embodiments, in the antisense strand, the nucleotide at position 4 is modified with 2′-O-methyl and the nucleotide at position 5 is a deoxynucleotide. In some embodiments, in the antisense strand, the nucleotide at position 4 is a deoxynucleotide and the nucleotide at position 5 is modified with 2′-O-methyl. In some embodiments, the antisense strand comprises two phosphorothioate linkages at both the 5′end and the 3′end. In some embodiments, the sense strand comprises nucleotides having the sequence of SEQ ID NO: 333, 541, 547, 557, or 569. In some embodiments, the antisense strand comprises nucleotides having the sequence of SEQ ID NO: 334, 542, 548, 558, or 570.

In one embodiment, provided is an siRNA comprising a sense strand and an antisense strand forming a double stranded duplex, wherein the sense strand comprises nucleotides having the sequence of SEQ ID NO: 333, and the antisense strand comprises nucleotides having the sequence of SEQ ID NO: 334. In one embodiment, provided is an siRNA comprising a sense strand and an antisense strand forming a double stranded duplex, wherein the sense strand comprises nucleotides having the sequence of SEQ ID NO: 335, and the antisense strand comprises nucleotides having the sequence of SEQ ID NO: 336. In another embodiment, provided is an siRNA comprising a sense strand and an antisense strand forming a double stranded duplex, wherein the sense strand comprises nucleotides having the sequence of SEQ ID NO: 339, and the antisense strand comprises nucleotides having the sequence of SEQ ID NO: 340. In another embodiment, provided is an siRNA comprising a sense strand and an antisense strand forming a double stranded duplex, wherein the sense strand comprises nucleotides having the sequence of SEQ ID NO: 341, and the antisense strand comprises nucleotides having the sequence of SEQ ID NO: 342. In another embodiment, provided is an siRNA comprising a sense strand and an antisense strand forming a double stranded duplex, wherein the sense strand comprises nucleotides having the sequence of SEQ ID NO: 507, and the antisense strand comprises nucleotides having the sequence of SEQ ID NO: 508. In another embodiment, provided is an siRNA comprising a sense strand and an antisense strand forming a double stranded duplex, wherein the sense strand comprises nucleotides having the sequence of SEQ ID NO: 509, and the antisense strand comprises nucleotides having the sequence of SEQ ID NO: 510. In another embodiment, provided is an siRNA comprising a sense strand and an antisense strand forming a double stranded duplex, wherein the sense strand comprises nucleotides having the sequence of SEQ ID NO: 511, and the antisense strand comprises nucleotides having the sequence of SEQ ID NO: 512. In another embodiment, provided is an siRNA comprising a sense strand and an antisense strand forming a double stranded duplex, wherein the sense strand comprises nucleotides having the sequence of SEQ ID NO: 513, and the antisense strand comprises nucleotides having the sequence of SEQ ID NO: 514. In another embodiment, provided is an siRNA comprising a sense strand and an antisense strand forming a double stranded duplex, wherein the sense strand comprises nucleotides having the sequence of SEQ ID NO: 515, and the antisense strand comprises nucleotides having the sequence of SEQ ID NO: 516. In another embodiment, provided is an siRNA comprising a sense strand and an antisense strand forming a double stranded duplex, wherein the sense strand comprises nucleotides having the sequence of SEQ ID NO: 521, and the antisense strand comprises nucleotides having the sequence of SEQ ID NO: 522. In another embodiment, provided is an siRNA comprising a sense strand and an antisense strand forming a double stranded duplex, wherein the sense strand comprises nucleotides having the sequence of SEQ ID NO: 527, and the antisense strand comprises nucleotides having the sequence of SEQ ID NO: 528. In another embodiment, provided is an siRNA comprising a sense strand and an antisense strand forming a double stranded duplex, wherein the sense strand comprises nucleotides having the sequence of SEQ ID NO: 531, and the antisense strand comprises nucleotides having the sequence of SEQ ID NO: 532. In another embodiment, provided is an siRNA comprising a sense strand and an antisense strand forming a double stranded duplex, wherein the sense strand comprises nucleotides having the sequence of SEQ ID NO: 533, and the antisense strand comprises nucleotides having the sequence of SEQ ID NO: 534. In another embodiment, provided is an siRNA comprising a sense strand and an antisense strand forming a double stranded duplex, wherein the sense strand comprises nucleotides having the sequence of SEQ ID NO: 541, and the antisense strand comprises nucleotides having the sequence of SEQ ID NO: 542. In another embodiment, provided is an siRNA comprising a sense strand and an antisense strand forming a double stranded duplex, wherein the sense strand comprises nucleotides having the sequence of SEQ ID NO: 547, and the antisense strand comprises nucleotides having the sequence of SEQ ID NO: 548. In another embodiment, provided is an siRNA comprising a sense strand and an antisense strand forming a double stranded duplex, wherein the sense strand comprises nucleotides having the sequence of SEQ ID NO: 557, and the antisense strand comprises nucleotides having the sequence of SEQ ID NO: 558. In another embodiment, provided is an siRNA comprising a sense strand and an antisense strand forming a double stranded duplex, wherein the sense strand comprises nucleotides having the sequence of SEQ ID NO: 567, and the antisense strand comprises nucleotides having the sequence of SEQ ID NO: 568. In another embodiment, provided is an siRNA comprising a sense strand and an antisense strand forming a double stranded duplex, wherein the sense strand comprises nucleotides having the sequence of SEQ ID NO: 569, and the antisense strand comprises nucleotides having the sequence of SEQ ID NO: 570.

In another aspect, provided herein is a composition comprising the siRNA agent of any one of the aspects or embodiments herein, and a carrier. In another aspect, provided herein is a method of inhibiting expression of MAPT gene in a cell or population of cells, the method comprising contacting the cell or population of cells with the siRNA of any one of the aspects or embodiments herein, or with the composition of any one of the aspects or embodiments herein.

### 5. BRIEF DESCRIPTION OF THE FIGURES

FIGS. 1A-1B demonstrate the effects of cholesterol-siRNA conjugates on MAPT mRNA expression in human iPSC-neurons. FIG. 1A depicts a graph showing the knockdown of MAPT mRNA in human iPSC-neurons by the cholesterol-siRNA conjugates, and FIG. 1B provides a table showing the IC50 (95% CI confidence interval) for the same cholesterol-siRNA conjugates.

FIGS. 2A-2B demonstrate the effects of lipid-siRNA conjugates on MAPT mRNA expression in human iPSC-neurons. FIG. 2A depicts a graph showing the knockdown of MAPT mRNA in human iPSC-neurons by the lipid-siRNA conjugates, and FIG. 2B provides a table showing the IC₅₀ (95% CI confidence interval) for the same lipid-siRNA conjugates.

FIG. 3 provides the chemical structures of 3′- and/or 5′-terminal lipid derivatives, and internal 2′-O-lipid derivatives. Also depicted is the structure of Chol4.

FIGS. 4A-4D demonstrate the effects of a lipid-siRNA conjugate on MAPT mRNA and Tau protein expression in the brain of Tau mouse model hTAU KI mice. FIGS. 4A-4B depict graphs showing the level (as % vehicle control) of human MAPT mRNA in the cortex and hippocampus, respectively, of the hTAU KI mice at different time points after the lipid-siRNA conjugate injection. FIGS. 4C-4D depict graphs showing the level (as % vehicle control) of human Tau protein in the cortex and hippocampus, respectively, of the hTAU KI mice at different time points after the lipid-siRNA conjugate injection.

FIGS. 5A-5B demonstrate the effects of different lipid-siRNA conjugates on MAPT mRNA expression in the brain of Tau mouse model hTAU KI mice. FIGS. 5A-5B depict graphs showing the level (as % vehicle control) of human MAPT mRNA in the cortex and hippocampus, respectively, of the hTAU KI mice at different time points after injection of the lipid-siRNA conjugates.

FIG. 6 demonstrates the efficiency of MAPT lipid-siRNA conjugates in knocking down MAPT mRNA in human iPSC-neurons. FIG. 6 depicts a graph showing MAPT mRNA expression (as % vehicle control) in human iPSC-neurons after incubation for 7 days with 1 μM and 250 nM of the MAPT lipid-siRNA conjugates.

### 6. DETAILED DESCRIPTION

The siRNA agents of the invention inhibit the expression of the MAPT gene. As used herein, the term “MAPT” gene, which is also known as MTBT1 gene, PPP1R103 gene, FTDP-17 gene, Tau-40 gene, MTBT2 gene, MAPTL gene, PPND gene, MSTD gene, Tau gene, and microtubule associated protein Tau gene, refers to the gene encoding a protein called microtubule associated protein Tau.

The human MAPT gene that encodes Tau protein is located on chromosome 17q23.1, spans ~150 kb and comprises 16 exons. 11 out of the 16 exons are expressed in the central nervous system (CNS). In human brain, six isoforms of Tau with 0, 1, or 2N-terminal repeats (0N, 1N, or 2N) and 3 or 4 microtubule-binding repeats (3R or 4R) are generated by alternative splicing of exons 2, 3, and 10. Bigger splicing variants that include exon 4a or exon 6 are mostly found in the peripheral nervous system, or in the spinal cord and skeletal muscle, respectively. Tau protein is predominantly expressed in the CNS neurons and located to the axons and dendrites. The half-life of Tau is reported as approximately 7 days in human iPSC-derived neuronal cultures and 23±6.4 days in human CNS (Sato, C., et al. (2018). “Tau Kinetics in Neurons and the Human Central Nervous System.” Neuron 98(4): 861-864). It is suggested that a fraction of newly synthesized Tau protein is C-terminally truncated and released from neurons with a delay of 3 days to the cerebrospinal fluid (CSF), and the estimated CSF Tau production rates from amyloid-negative and -positive cohorts are 22.9±7.2 pg/mL/day and 27.8±7.0 pg/mL/day, respectively (Sato, C., et al. (2018). “Tau Kinetics in Neurons and the Human Central Nervous System.” Neuron 98(4): 861-864).

As used herein, the term “small interfering RNA agent” or “siRNA agent” means an RNA or RNA-like (e.g., chemically modified RNA) oligonucleotide molecule that contains both a sense and an anti-sense strand. In some embodiments, the siRNA sense strand is 30, 29, 28, 27, 26, 25, 24, 23, 22, 21, 20, 19, 18 or 17 nucleotides in length. In some embodiments, the siRNA anti-sense strand is 30, 29, 28, 27, 26, 25, 24, 23, 22, 21, 20, 19, 18 or 17 nucleotides in length. The sense strand and/or antisense strand of a siRNA agent may comprise another moiety (e.g., a lipid moiety). For example, a lipid moiety may be incorporated into the sense strand of a siRNA agent. The sense strand and/or antisense strand of a siRNA agent may be linked or conjugated, directly or indirectly, to another moiety (e.g., a lipid moiety). For example, the sense strand of the siRNA agent may be linked or conjugated, directly or indirectly, to another moiety (e.g., a lipid moiety). In a specific embodiment, a siRNA agent is a double-stranded RNA or RNA-like (e.g., chemically modified RNA) oligonucleotide comprising a sense stand and an antisense strand forming a double-stranded region. The double-stranded region may be the entire length of the sense strand, antisense strand, or both. Alternatively, the double-stranded region may be less than the entire length of the sense strand, antisense strand, or both. The double-stranded region may be the result of the antisense strand being fully complementary, partially complementary, or substantially complementary to the sense strand. In a specific embodiment, the antisense strand of a siRNA agent is partially complementary to a target RNA transcript. In another specific embodiment, the antisense strand of a siRNA agent is substantially complementary to a target RNA transcript. In another specific embodiment, the antisense strand of a siRNA agent is fully complementary to a target RNA transcript.

The two strands forming the double-stranded region or duplex structure may be different portions of one larger RNA molecule, or they may be separate RNA molecules.

Where the two substantially complementary strands of a siRNA agent are comprised by separate RNA molecules, those molecules need not, but can be covalently connected. In certain embodiments where the two strands are connected covalently by means other than a contiguous chain of nucleotides between the 3′-end of one strand and the 5′-end of the respective other strand forming the duplex structure, the connecting structure is referred to as a “linker”. The RNA strands of a siRNA agent may have the same or a different number of nucleotides. In some embodiments, one or both strands of a siRNA agent comprise an overhang. In other embodiments, the siRNA agent is blunt ended.

In a specific embodiment, a siRNA agent described herein mediates messenger RNA (mRNA) degradation or inhibition of translation of the mRNA in a sequence-specific manner. In specific embodiments, a siRNA agent described herein inhibits MAPT gene expression via an RNA-induced silencing complex (RISC) pathway

As used herein, term “complementary,” when used to describe a first nucleotide sequence (e.g., a sense strand of a siRNA agent, or targeted sequence) in relation to a second nucleotide sequence (e.g., antisense strand of a siRNA agent, or a single-stranded antisense oligonucleotide), means the ability of an oligonucleotide or polynucleotide including the first nucleotide sequence to hybridize (form base pair hydrogen bonds under mammalian physiological conditions (or similar conditions in vitro)) and form a duplex or double helical structure under certain conditions with an oligonucleotide or polynucleotide including the second nucleotide sequence. Complementary sequences include Watson-Crick base pairs or non-Watson-Crick base pairs and include natural or modified nucleotides or nucleotide mimics, at least to the extent that the above hybridization requirements are fulfilled. Sequence identity or complementarity is independent of modification. For example, fA and mA are complementary to U (or T) and identical to A for the purposes of determining identity or complementarity.

As used herein, the term “fully complementary” in the context of two nucleotide sequences means that all (100%) of the bases in a contiguous sequence of a first nucleotide sequence will hybridize to the same number of bases in a contiguous sequence of a second nucleotide sequence to form a duplex. Where two nucleotide sequences are designed to form, upon hybridization, one or more single stranded overhangs, such overhangs shall not be regarded as mismatches with regard to the determination of complementarity. For example, a siRNA agent comprising one oligonucleotide 21 nucleotides in length and another oligonucleotide 23 nucleotides in length, wherein the 23 nucleotides oligonucleotide comprises a sequence of 21 nucleotides that is fully complementary to the 21 nucleotides oligonucleotide, are considered “fully complementary” for the purposes described herein. In a specific embodiment, two nucleotide sequences are “fully complementary” when all (100%) of the bases of a first nucleotide sequence hybridize to all (100%) of the bases in a second nucleotide sequence to form a duplex. In one specific embodiment, the two nucleotide sequences hybridize under stringent conditions. In another specific embodiment, the two nucleotide sequences hybridize under very stringent conditions.

As used herein, the term “partially complementary” in the context of two nucleotide sequences means that at least 65% but less than 80% of the bases in a contiguous sequence of a first nucleotide sequence will hybridize to the same number of bases in a contiguous sequence of a second nucleotide sequence to form a duplex. In one specific embodiment, the two nucleotide sequences hybridize under stringent conditions. In another specific embodiment, the two nucleotide sequences hybridize under very stringent conditions.

As used herein, the term “substantially complementary” in the context of two nucleotide sequences means that at least 80% but less than 100% of the bases in a contiguous sequence of a first nucleotide sequence will hybridize to the same number of bases in a contiguous sequence of a second nucleotide sequence to form a duplex. In some embodiments, two nucleotide sequences are substantially complementary when at least 85%, at least 90%, at least 91%, at least 92%, at least 93%, at least 94%, at least 95%, at least 96%, at least 97%, at least 98%, but less than 100% of the bases in a contiguous sequence of a first oligonucleotide will hybridize to the same number of bases in a contiguous sequence of a second oligonucleotide to form a duplex. In one specific embodiment, the two nucleotide sequences hybridize under stringent conditions. In another specific embodiment, the two nucleotide sequences hybridize under very stringent conditions.

As used herein, the terms “about” and “approximate” when referring to a numerical value encompass the recited numerical value and variations within +/−20%. For example, about 20% would encompass 16% to 24% and values in between, including 20%. In one embodiment, the terms “about” and “approximate” when referring to a numerical value encompass the recited numerical value and variations within +1-15%. In another embodiment, the terms “about” and “approximate” when referring to a numerical value encompass the recited numerical value and variations within +/−10%. In another embodiment, the terms “about” and “approximate” when referring to a numerical value encompass the recited numerical value and variations within +/−5%.

As used herein, the term “stringent” when referring to hybridization means that under “stringent conditions”, or “stringent hybridization conditions”, a first nucleotide sequence will hybridize to a second nucleotide, with minimal hybridization to other sequences. In a specific embodiment, an antisense sequence will hybridize under stringent conditions to its target sequence, with minimal targeting to other sequences. Stringent conditions are sequence dependent (e.g., sequence length, complementarity), and vary under different environmental parameters (e.g., assay conditions, physiological environment). An example of stringent hybridization conditions may include: 400 mM NaCl, 40 mM PIPES pH 6.4, 1 mM EDTA, 50° C. or 70° C. for 12-16 hours followed by washing. A skilled person will understand that variations in the stringency of hybridization are inherently described.

As used herein, the term “very stringent” when referring to hybridization means that under “very stringent conditions” or “very stringent hybridization conditions”, a first nucleotide sequence will only be observed to hybridize to a second nucleotide. In a specific embodiment, an antisense sequence will be observed to only hybridize to its target sequence under very stringent conditions. Also, very stringent conditions may not allow hybridization to occur between partially complementary sequences. Very stringent conditions are sequence dependent (e.g., sequence length, complementarity), and vary under different environmental parameters (e.g., assay conditions, physiological environment). Very stringent conditions may include a higher temperature, lower ionic strength, and/or shorter reaction time compared to stringent conditions under the same circumstance. For example, very stringent conditions may include a hybridization temperature of about 71° C., about 72° C., about 73° C., about 74° C., about 75° C., about 76° C., about 77° C., about 78° C., about 79° C., about 80° C., or higher. A skilled person will understand that variations in the stringency of hybridization are inherently described.

As used herein, “target sequence” refers to a contiguous portion of a nucleotide sequence of a RNA molecule formed during the transcription of a MAPT gene, including mRNA that is a product of RNA processing of a primary transcription product (e.g., MAPT mRNA resulting from alternate splicing). In one embodiment, the contiguous portion of the nucleotide sequence is at least long enough to serve as a substrate for RNAi-directed cleavage at or near that portion of the nucleotide sequence of an mRNA molecule formed during the transcription of a MAPT gene. In specific embodiments, the target sequence is about 15-30 nucleotides in length. For example, the target sequence can be from about 15-30 nucleotides, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length. In certain embodiments, the target sequence is 19-25 nucleotides in length. In some embodiments, the target sequence is 21-23 nucleotides in length. Ranges and lengths intermediate to the above recited ranges and lengths are also contemplated to be part of the disclosure.

As used herein, the phrase “nucleotide sequence corresponding to any one of the antisense strand nucleotide sequences”, “nucleotide sequence corresponding to the antisense strand nucleotide sequence”, “nucleotide sequence corresponding to any one of the sense strand nucleotide sequences”, “nucleotide sequence corresponding to the sense strand nucleotide sequence”, “nucleotide sequence corresponding to any one of the nucleotide sequences”, or “nucleotide sequence corresponding to any one of SEQ ID NOS” refers to an oligonucleotide comprising a chain of nucleotides comprising the recited unmodified nucleotides, or one or more modified nucleotides, or one or more conjugated moieties (e.g., a moiety described herein, such as, e.g., a lipid, or a modified nucleotide conjugated to a moiety described herein). A skilled person is aware that the recited unmodified nucleotide may be replaced by other moieties without substantially altering the base pairing properties of an oligonucleotide comprising a nucleotide bearing such replacement moiety. For example, a nucleotide comprising inosine as its base can base pair with nucleotides containing adenine, cytosine, or uracil. Thus, a nucleotide containing uracil, guanine, or adenine can be replaced by a nucleotide containing, for example, inosine. In another example, adenine and cytosine may be replaced with guanine and uracil, respectively to form G-U Wobble base pairing with the target mRNA.

As used herein, the phrase “non-naturally occurring small interfering ribonucleic acid (siRNA) agent”, “non-naturally occurring small interfering ribonucleic acid agent”, or “non-naturally occurring siRNA agent” refers to a siRNA agent that is not found in nature. The non-naturally occurring siRNA may contain one or more modified nucleotides.

As used herein, the term “overhang” in the context of a 5′ or 3′ nucleotide overhang refers to at least one unpaired nucleotide that protrudes from the duplex structure of a siRNA agent. For example, when a 3′-end of one strand of a siRNA agent extends beyond the 5′-end of the other strand, or vice versa, there is a nucleotide overhang. In some embodiments, the overhang is present at the 3′-end of the sense strand, antisense strand, or both strands. In one embodiment, the 3′-overhang is present in the antisense strand. In another embodiment, the 3′-overhang is present in the sense strand. In some embodiments, the overhang is present at the 5′-end of the sense strand, antisense strand, or both strands. In one embodiment, the 5′-overhang is present in the antisense strand. In another embodiment, the 5′-overhang is present in the sense strand. The overhangs may be due to one strand being longer than the other, or the result of two strands of the same length being staggered. In some embodiments, the overhang forms a mismatch with the target sequence. In other embodiments, the overhang is complementary to the gene sequences being targeted. The nucleotides in the overhang region of a siRNA agent may each independently be a modified or unmodified nucleotide (e.g., 2′-fluoro-modified nucleotide, 2′-O-methyl modified nucleotide, deoxynucleotide, or a combination thereof).

In some embodiments, the 5′- or 3′—overhangs of the sense strand or antisense strand of a siRNA agent are phosphorylated. In certain embodiments, the 5′- or 3′-overhangs of the sense strand and the antisense strand of a siRNA agent are phosphorylated. In some embodiments, the overhang region(s) contains two nucleotides having a phosphorothioate between the two nucleotides, and those two nucleotides can be the same or different.

In some embodiments, a siRNA agent contains only a single overhang, which can strengthen the interference activity of the siRNA agent, without affecting its overall stability. For example, the single-stranded overhang may be located at the 3′-terminal end of the sense strand of the siRNA agent, or, alternatively, at the 3′-terminal end of the antisense strand of the siRNA agent. The siRNA agent may also have a blunt end, located at the 5′-end of the antisense strand (or the 3′-end of the sense strand) or vice versa. In some embodiments, the antisense strand of the siRNA agent has a nucleotide overhang at the 3′-end, and the 5′-end is blunt ended.

In some embodiments, one strand of a siRNA agent comprises a 5′-end, a 3′-end, or both a 5′-end and a 3′-end overhang of at least 1 nucleotide, at least 2 nucleotides, or at least 3 nucleotides. In certain embodiments, one strand of a siRNA agent comprises a 5′-end, a 3′-end, or both a 5′-end and a 3′-end overhang of at least 1 nucleotide, at least 2 nucleotides, or at least 3 nucleotides, but no more than 5 nucleotides. In some embodiments, one strand of a siRNA agent comprises a 5′-end, a 3′-end, or both a 5′-end and a 3′-end overhang of 1 nucleotide, 2 nucleotides, or 3 nucleotides. In certain embodiments, one strand of a siRNA agent comprises a 5′-end, a 3′-end, or both a 5′-end and a 3′-end overhang of 1 to 2 nucleotides, 1 to 3 nucleotides, 1 to 4 nucleotides, or 1 to 5 nucleotides. In some embodiments, one strand of a siRNA agent comprises a 5′-end, a 3′-end, or both a 5′-end and a 3′-end overhang of 2 to 3 nucleotides, 2 to 4 nucleotides, or 2 to 5 nucleotides. In certain embodiments, one strand of a siRNA agent comprises a 5′-end, a 3′-end, or both a 5′-end and a 3′-end overhang of 3 to 4 nucleotides, or 4 to 5 nucleotides. The strand may be an antisense strand or a sense strand. A nucleotide overhang may comprise or consist of a nucleotide analog or a nucleoside analog.

In some embodiments, each strand of a siRNA agent comprises a 5′-end, a 3′-end, or both a 5′-end and a 3′-end overhang of at least 1 nucleotide, at least 2 nucleotides, or at least 3 nucleotides. In certain embodiments, each strand of a siRNA agent comprises a 5′-end, a 3′-end, or both a 5′-end and a 3′-end overhang of at least 1 nucleotide, at least 2 nucleotides, or at least 3 nucleotides, but no more than 5 nucleotides. In some embodiments, each strand of a siRNA agent comprises a 5′-end, a 3′-end, or both a 5′-end and a 3′-end overhang of 1 nucleotide, 2 nucleotides, or 3 nucleotides. In certain embodiments, each strand of a siRNA agent comprises a 5′-end, a 3′-end, or both a 5′-end and a 3′-end overhang of 1 to 2 nucleotides, 1 to 3 nucleotides, 1 to 4 nucleotides, or 1 to 5 nucleotides. In some embodiments, each strand of a siRNA agent comprises a 5′-end, a 3′-end, or both a 5′-end and a 3′-end overhang of 2 to 3 nucleotides, 2 to 4 nucleotides, or 2 to 5 nucleotides. In certain embodiments, each strand of a siRNA agent comprises a 5′-end, a 3′-end, or both a 5′-end and a 3′-end overhang of 3 to 4 nucleotides, or 4 to 5 nucleotides. A nucleotide overhang may comprise or consist of a nucleotide analog or a nucleoside analog.

As used herein, the term “blunt” or “blunt ended” in the context of a siRNA agent means that there are no unpaired nucleotides or nucleotide analogs at a given terminal end of a siRNA agent, i.e., no nucleotide overhang. In some embodiments, one end of a siRNA agent is blunt ended. In other words, the 5′-end of one strand and the 3′-end of the other strand do not include an unpaired nucleotide or nucleotide analog. In some embodiments, both ends of a siRNA agent are blunt ended. In other words, there is no nucleotide overhang at either end of the siRNA agent.

As used herein, term “antisense strand” or “guide strand” in the context of a siRNA agent refers to the strand which includes a region that is complementary to a target sequence.

As used herein, the term “sense strand” or “passenger strand” in the context of a siRNA agent refers to the strand of a siRNA agent that includes a region that is complementary to a region of the antisense strand.

As used herein, the term “modified” in the context of a nucleobase of a siRNA agent, refers to a nucleobase not found in nature in an RNA molecule. Naturally occurring RNA sequences include purine bases adenine (A) and guanine (G), and pyrimidine bases cytosine (C) and uracil (U). See Section 6.2, infra, for examples of modified nucleobases.

As used herein, the term “modified” in the context of a nucleotide of a siRNA agent, refers to a nucleotide not found in nature in an RNA molecule. See Section 6.2, infra, for examples of modified nucleotides.

As used herein, and unless otherwise specified, the term “isomer” refers to different compounds that have the same molecular formula. “Stereoisomers” are isomers that differ only in the way the atoms are arranged in space. “Atropisomers” are stereoisomers from hindered rotation about single bonds. “Enantiomers” are a pair of stereoisomers that are non-superimposable mirror images of each other. A mixture of a pair of enantiomers in any proportion can be known as a “racemic” mixture. “Diastereoisomers” are stereoisomers that have at least two asymmetric atoms, but which are not mirror-images of each other.

“Stereoisomers” can also include E and Z isomers, or a mixture thereof, and cis and trans isomers or a mixture thereof. In certain embodiments, a compound described herein is isolated as either the E or Z isomer. In other embodiments, a compound described herein is a mixture of the E and Z isomers.

It should be noted that if there is a discrepancy between a depicted structure and a name for that structure, the depicted structure is to be accorded more weight.

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments.

As used herein, the term “or” is to be interpreted as an inclusive “or” meaning any one or any combination. Therefore, “A, B, or C” means any of the following: A; B; C; A and B; A and C; B and C; A, B, and C. An exception to this definition will occur only when a combination of elements, functions, steps or acts are in some way inherently mutually exclusive.

As used herein, the phrase “and/or” as used in a phrase such as “A and/or B” herein is intended to include both A and B; A or B; A (alone); and B (alone). Likewise, the phrase “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### 6.1 Nucleotide Sequences

Provided herein are siRNA agents which inhibit (e.g., partially or completely) the expression of a MAPT gene (e.g., a human MAPT gene). In a specific embodiment, a siRNA described herein inhibits expression of a MAPT gene (e.g., a human MAPT gene) by at least about 25%, at least about 30%, at least about 40%, at least about 50%, at least about 60% relative to a negative control (e.g., PBS or siRNA directed to an RNA molecule formed during the transcription of a gene other than MAPT) in an assay known to one of skill in the art, or described herein (e.g., in Section 6.8, infra, or Section 7, infra). In another specific embodiment, a siRNA described herein inhibits expression of a MAPT gene (e.g., a human MAPT gene) by at least about 70%, at least 75%, at least 80%, or at least 85% relative to a negative control (e.g., PBS or siRNA directed to an RNA molecule formed during the transcription of a gene other than MAPT) in an assay known to one of skill in the art, or described herein (e.g., in Section 6.8, infra, or Section 7, infra). In another specific embodiment, a siRNA described herein inhibits expression of a MAPT gene (e.g., a human MAPT gene) by at least about 90%, at least 95%, at least 96%, at least 97%, or at least 98% relative to a negative control (e.g., PBS or siRNA directed to an RNA molecule formed during the transcription of a gene other than MAPT) in an assay known to one of skill in the art, or described herein (e.g., in Section 6.8, infra, or Section 7, infra). In another specific embodiment, a siRNA described herein inhibits expression of a MAPT gene (e.g., a human MAPT gene) by 25% to 50%, 50% to 75%, 75% to 85%, 85% to 95%, 90% to 95%, or 95% to 99% relative to a negative control (e.g., PBS or siRNA directed to an RNA molecule formed during the transcription of a gene other than MAPT) in an assay known to one of skill in the art, or described herein (e.g., in Section 6.8, infra, or Section 7, infra). In another specific embodiment, a siRNA described herein inhibits expression of a MAPT gene (e.g., a human MAPT gene) by 100% relative to a negative control (e.g., PBS or siRNA directed to an RNA molecule formed during the transcription of a gene other than MAPT) in an assay known to one of skill in the art, or described herein (e.g., in Section 6.8, infra, or Section 7, infra). In a specific embodiment, inhibition of MAPT gene (e.g., a human MAPT gene) expression assessed using an in vitro assay described in Section 7, infra. In a specific embodiment, a siRNA agent described herein exhibits knockdown efficiency in vitro or ex vivo, such as, e.g., described in Section 7, infra. In a specific embodiment, a siRNA agent described herein exhibits knockdown efficiency in vivo in a mouse model, such as, e.g., described in Section 7, infra. In specific embodiments, a siRNA agent described herein exhibits one, two, three, or more of the properties of an RNA molecule described in Section 7, infra.

In a specific embodiment, a siRNA agent described herein inhibits the expression of a MAPT gene (e.g., human MAPT gene) in neurons (e.g., human iPSC-neurons) at an IC₅₀ of 10 nM to 200 nM in an assay described herein (e.g., Section 7, infra). In a specific embodiment, a siRNA agent described herein inhibits the expression of a MAPT gene (e.g., human MAPT gene) in neurons (e.g., human iPSC-neurons) at an IC₅₀ of 10 nM to 100 nM in an assay described herein (e.g., Section 7, infra). In a specific embodiment, a siRNA agent described herein inhibits the expression of a MAPT gene (e.g., human MAPT gene) in neurons (e.g., human iPSC-neurons) at an IC₅₀ of 10 nM to 50 nM in an assay described herein (e.g., Section 7, infra).

In a specific embodiment, a siRNA agent described herein is stable in vitro or ex vivo in one, two, or all of the following: mouse brain homogenates, human liver lysosomes and rat liver tritosomes. The stability of such a siRNA agent may be assessed using a method known to one of skill in the art, or as described herein (e.g., in Section 6.8, or Section 7, infra).

In a specific embodiment, a siRNA agent is a double-stranded RNA or RNA-like (e.g., chemically modified RNA) oligonucleotide comprising a sense stand and an antisense strand, which anneal to form a double-stranded region or duplex. The antisense strand includes a region that is substantially complementary, and generally fully complementary, to the target sequence. The sense strand includes a region that is complementary to the antisense strand, such that the two strands hybridize and form a duplex structure when combined under suitable conditions. As described herein and as known in the art, the complementary sequences of a siRNA agent can also be contained as self-complementary regions of a single nucleic acid molecule, as opposed to being on separate oligonucleotides. In a specific embodiment, the annealment of the sense strand and antisense strand sequences form a duplex that is stable as assessed by a melting temperature (Tm) of about 40° C., about 41° C., about 42° C., about 43° C., about 44° C., about 45° C., or higher. See, e.g., Section 6.8 below for melting temperature assays.

The sense strand and the antisense strand of a siRNA agent described herein may be the same length or different lengths. The sense strand and the antisense strand of a siRNA agent described herein can each be 15 to 30 nucleotides in length. In specific embodiments, the sense strand and the antisense strand of a siRNA agent described herein are each no more than 30 nucleotides in length. In some embodiments, the sense strand and the antisense strand of a siRNA agent described herein are each independently 15 to 30 nucleotides in length. In certain embodiments, the sense and the antisense strand of a siRNA agent described herein are each independently 16 to 30 nucleotides in length. In some embodiments, the sense and the antisense strand of a siRNA agent described herein are each independently 17 to 30 nucleotides in length. In certain embodiments, the sense and the antisense strand of a siRNA agent described herein are each independently 18 to 30 nucleotides in length. In some embodiments, the sense and the antisense strand of a siRNA agent described herein are each independently 19 to 30 nucleotides in length. In certain embodiments, the sense and the antisense strand of a siRNA agent described herein are each independently 20 to 30 nucleotides in length. In certain embodiments, the sense and the antisense strand of a siRNA agent described herein are each independently 21 to 30 nucleotides in length.

In some embodiments, the sense strand and the antisense strand of a siRNA agent described herein are each independently 15 to 25 nucleotides in length. In certain embodiments, the sense and the antisense strand of a siRNA agent described herein are each independently 16 to 25 nucleotides in length. In some embodiments, the sense and the antisense strand of a siRNA agent described herein are each independently 17 to 25 nucleotides in length. In certain embodiments, the sense and the antisense strand of a siRNA agent described herein are each independently 18 to 25 nucleotides in length. In some embodiments, the sense and the antisense strand of a siRNA agent described herein are each independently 19 to 25 nucleotides in length. In certain embodiments, the sense and the antisense strand of a siRNA agent described herein are each independently 20 to 25 nucleotides in length. In certain embodiments, the sense and the antisense strand of a siRNA agent described herein are each independently 21 to 25 nucleotides in length.

In some embodiments, the sense strand and the antisense strand of a siRNA agent described herein are each independently 19 to 23 nucleotides in length. In certain embodiments, the sense and the antisense strand of a siRNA agent described herein are each independently 19 to 22 nucleotides in length. In some embodiments, the sense and the antisense strand of a siRNA agent described herein are each independently 19 to 21 nucleotides in length. In certain embodiments, the sense and the antisense strand of a siRNA agent described herein are each independently 19 nucleotides in length. In some embodiments, the sense and the antisense strand of a siRNA agent described herein are each independently 20 nucleotides in length. In certain embodiments, the sense and the antisense strand of a siRNA agent described herein are each independently 21 nucleotides in length. In certain embodiments, the sense and the antisense strand of a siRNA agent described herein are each independently 22 nucleotides in length.

The double stranded region formed by hybridization of the antisense strand and the sense strand of a siRNA agent described herein may be the entire length of both strands. For example, the antisense strand of a siRNA agent described herein may be 19 nucleotides in length, the sense strand of the siRNA agent may be 19 nucleotides in length, and the double strand region formed by hybridization of the strands is 19 nucleotides. In another example, the antisense strand of a siRNA agent described herein may be 21 nucleotides in length, the sense strand of the siRNA agent may be 21 nucleotides in length, and the double strand region formed by hybridization of the strands is 21 nucleotides. The double stranded region formed by hybridization of the antisense strand and the sense strand of a siRNA agent described herein may be less than entire length of one or both strands. For example, the antisense strand of a siRNA agent described herein may be 19 nucleotides in length, the sense strand of the siRNA agent may be 21 nucleotides in length, and the double strand region formed by hybridization of the strands is 19 nucleotides. In another example, the antisense strand of a siRNA agent described herein may be 21 nucleotides in length, the sense strand of the siRNA agent may be 19 nucleotides in length, and the double strand region formed by hybridization of the strands is 19 nucleotides. In another example, the antisense strand of a siRNA agent described herein may be 21 nucleotides in length, the sense strand of the siRNA agent may be 21 nucleotides in length, and the double strand region formed by hybridization of the strands is 19 nucleotides. In a specific embodiment, the double-strand region of a siRNA agent described herein is long enough to serve as a substrate for the Dicer enzyme.

In certain embodiments, the double strand region of a siRNA agent described herein is 15 to 30 base pairs in length. For example, the double strand region of a siRNA agent described herein may be 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs in length. In some embodiments, the double strand region of a siRNA agent described herein is 15 to 25 base pairs in length. In certain embodiments, the double strand region of a siRNA agent described herein is 16 to 25 base pairs in length. In some embodiments, the double strand region of a siRNA agent described herein is 17 to 25 base pairs in length. In some embodiments, the double strand region of a siRNA agent described herein is 18 to 25 base pairs in length. In certain embodiments, the double strand region of a siRNA agent described herein is 19 to 25 base pairs in length. In some embodiments, the double strand region of a siRNA agent described herein is 20 to 25 base pairs in length. In some embodiments, the double strand region of a siRNA agent described herein is 21 to 25 base pairs in length. In some embodiments, the double strand region of a siRNA agent described herein is 19 to 23 base pairs in length. In certain embodiments, the double strand region of a siRNA agent described herein is 19 to 22 base pairs in length. In some embodiments, the double strand region of a siRNA agent described herein is 19 to 21 base pairs in length. In certain embodiments, the double strand region of a siRNA agent described herein is 15, 16, 17, 18, 19, 20, 21, 22, 23, 24, or 25 base pairs in length. In specific embodiments, the double strand region of a siRNA agent described herein is 19, 20, 21, 22 or 23 base pairs in length. Ranges and lengths intermediate to the above recited ranges and lengths are also contemplated to be part of the disclosure.

In some embodiments, a siRNA agent described herein is blunt ended. In other embodiments, the sense strand or the antisense strand of a siRNA agent described herein comprises an overhang. The overhang may be at the 5′ end, the 3′ end, or both the 5′ and 3′ end of either the sense strand or antisense strand of the siRNA agent. In some embodiments, the sense strand and the antisense strand of a siRNA agent described herein comprise an overhang. The overhang of the sense strand may be at the 5′ end of the sense strand, and the overhang of the antisense strand may be at the 3′ end of the antisense strand. Alternatively, the overhang of the sense strand may be at the 3′ end of the sense strand, the overhang of the antisense strand may be at the 5′ end of the antisense strand. In certain embodiments, the overhang of a strand of a siRNA agent comprises at least 1 nucleotide. In some embodiments, the overhang of a strand of a siRNA agent comprises at least 2 nucleotides. In certain embodiments, the overhang of a strand of a siRNA agent comprises at least 3 nucleotides. In some embodiments, the overhang of a strand of a siRNA agent comprises at least 4 nucleotides. In certain embodiments, the overhang of a strand of a siRNA agent comprises at least 5 nucleotides. In some embodiments, the overhang of a strand of a siRNA agent comprises 1 nucleotide, 2 nucleotides, 3 nucleotides, 4 nucleotide, 5 nucleotides, or more. In certain embodiments, the overhang of a strand of a siRNA agent comprises 1 to 5 nucleotides, 1 to 4 nucleotides, 1 to 3 nucleotides, or 1 to 2 nucleotides. In some embodiments, the overhang of a strand of a siRNA agent comprises 2 to 5 nucleotides, 2 to 4 nucleotides, 2 to 3 nucleotides, 3 to 4 nucleotides, 3 to 4 nucleotides, or 4 to 5 nucleotides.

In certain embodiments, the sense strand of a siRNA agent described herein is 19 nucleotides in length and the antisense strand of the siRNA agent is 21 nucleotides in length. In some embodiments, the sense strand of a siRNA agent described herein is 20 nucleotides in length and the antisense strand of the siRNA agent is 21 nucleotides in length. In certain embodiments, the sense strand of a siRNA agent described herein is 21 nucleotides in length and the antisense strand of the siRNA agent is 21 nucleotides in length. In some embodiments, the sense strand of a siRNA agent described herein is 21 nucleotides in length and the antisense strand of the siRNA agent is 19 nucleotides in length. In certain embodiments, the sense strand of a siRNA agent described herein is 21 nucleotides in length and the antisense strand of the siRNA agent is 20 nucleotides in length. In some embodiments, the sense strand of a siRNA agent described herein is 19 nucleotides in length and the antisense strand of the siRNA agent is 19 nucleotides in length. In some embodiments, the sense strand of a siRNA agent described herein is 20 nucleotides in length and the antisense strand of the siRNA agent is 20 nucleotides in length.

In one embodiment, provided herein is a siRNA agent comprising a sense strand and an antisense strand forming a double stranded region, wherein the sense strand comprises a nucleotide sequence corresponding to the nucleotide sequence of any one of the sense strand nucleotide sequences in Table 1.

In one embodiment, provided herein is a siRNA agent comprising a sense strand and an antisense strand forming a double stranded region, wherein the antisense strand comprises a nucleotide sequence corresponding to the nucleotide sequence of any one of the antisense strand nucleotide sequences in Table 1.

In one embodiment, provided herein is a siRNA agent comprising a sense strand and an antisense strand forming a double stranded region, wherein the antisense strand comprises a nucleotide sequence corresponding to the nucleotide sequence of the antisense strand nucleotide sequence in a row in Table 1, and wherein the sense strand comprises a nucleotide sequence corresponding to the nucleotide sequence of the sense strand nucleotide sequence in the same row in Table 1. In specific embodiments, provided herein is a siRNA agent comprising a sense strand and an antisense strand forming a double stranded region, wherein the antisense strand comprises a nucleotide sequence corresponding to the nucleotide sequence of the antisense strand nucleotide sequence of UM25, UM28, UM96, UM165, or UM177 in Table 1, and the sense strand comprises a nucleotide sequence corresponding to the nucleotide sequence of the sense strand nucleotide sequence of UM25, UM28, UM96, UM165, or UM177 in Table 1, respectively.

In certain embodiments, provided herein is a siRNA agent comprising a sense strand and an antisense strand forming a double stranded region, wherein the antisense strand comprises a nucleotide sequence corresponding to the nucleotide sequence of any one of the antisense strand nucleotide sequences in Table 1, and the sense strand comprises a nucleotide sequence partially complementary to the antisense strand. In some embodiments, provided herein is a siRNA agent comprising a sense strand and an antisense strand forming a double stranded region, wherein the antisense strand comprises a nucleotide sequence corresponding to the nucleotide sequence of any one of the antisense strand nucleotide sequences in Table 1, and the sense strand comprises a nucleotide sequence substantially complementary to the antisense strand. In certain embodiments, provided herein is a siRNA agent comprising a sense strand and an antisense strand forming a double stranded region, wherein the antisense strand comprises a nucleotide sequence corresponding to the nucleotide sequence of any one of the antisense strand nucleotide sequences in Table 1, and the sense strand comprises a nucleotide sequence fully complementary to the antisense strand.

In certain embodiments, provided herein is a siRNA agent comprising a sense strand and an antisense strand forming a double stranded region, wherein the sense strand comprises a nucleotide sequence corresponding to the nucleotide sequence of any one of the sense strand nucleotide sequences in Table 1, and the antisense strand comprises a nucleotide sequence partially complementary to the sense strand. In some embodiments, provided herein is a siRNA agent comprising a sense strand and an antisense strand forming a double stranded region, wherein the sense strand comprises a nucleotide sequence corresponding to the nucleotide sequence of any one of the sense strand nucleotide sequences in Table 1, and the antisense strand comprises a nucleotide sequence substantially complementary to the sense strand. In certain embodiments, provided herein is a siRNA agent comprising a sense strand and an antisense strand forming a double stranded region, wherein the sense strand comprises a nucleotide sequence corresponding to the nucleotide sequence of any one of the sense strand nucleotide sequences in Table 1, and the antisense strand comprises a nucleotide sequence fully complementary to the sense strand.

In some embodiments, a siRNA agent does not comprise, e.g., chemical modifications or conjugations known in the art and described herein. See, e.g., Section 6.2, infra, and Section 7, infra, for siRNA agents comprising chemical modifications or conjugations. In specific embodiments, a siRNA agent comprises, e.g., chemical modifications or conjugations known in the art and described herein. In a specific embodiment, a siRNA agent comprises one or more chemical modifications and/or conjugations described herein (e.g., in Section 6.2, infra, and/or Section 7, infra In certain embodiments, the nucleobase of a nucleotide is modified. In some embodiments, the sugar of the nucleotide is modified. In certain embodiments, the phosphate backbone of the nucleotide is modified.

The siRNA agents may contain only naturally occurring nucleotides, or may contain one or more modified nucleotides. In certain embodiments, the nucleobase of a nucleotide is modified. In some embodiments, the sugar of the nucleotide is modified. In certain embodiments, the phosphate backbone of the nucleotide is modified.

In specific embodiments, the modified nucleotides comprise one or more modified nucleotides described herein (e.g., in Section 6.2, infra, or Section 7, infra). In specific embodiments, the modified nucleotides are selected from the group consisting of a 2′O-methyl modified nucleotide, a deoxy-nucleotide, a 2′-fluoro modified nucleotide, a 2′-O-methyl-uridine, a 3′-O-methyl modified nucleotide, a 3′-O-methyl modified nucleotide with 2′-5′ linked phosphate, an inverted abasic nucleotide, a nucleotide comprising S-glycol nucleic acid (GNA), an unlocked nucleotide, a 5′-vinylphosphonate-2′-O-methyl-uridine, a cis-cyclobutyl phosphonate modified nucleotide, a 5′-cis-cyclobutyl phosphonate-2′-O-methyl modified nucleotide, a (L)-α-threofuranosyl modified nucleotide, and combinations thereof.

In specific embodiments, the antisense strand of a siRNA agent described herein comprises at least one modified internucleoside linkage.

In specific embodiments, the sense strand of a siRNA agent described herein comprises at least one modified internucleoside linkage.

In specific embodiments, the antisense strand and the sense strand of a siRNA agent described herein each comprise at least one modified internucleoside linkage In a specific embodiment, the modified internucleoside linkages include phosphorothioate.

In specific embodiments, siRNA agent described herein comprises one or more of the following: a phosphorothioate (PS) linkage, 2′-fluororibose (2′-F), 2′-methoxyribose (2′-OMe), deoxyribose, 2′-O-(2-methoxyethyl)ribose (2′-MOE), an inverted linkage (inverted abasic site, InvAb), unlocked nucleic acid (UNA), and glycol nucleic acid (GNA).

In specific embodiments, a siRNA agent described herein exhibits stability as assessed by an assay described herein (e.g., in Section 6.8, infra, or Section 7, infra), or known to one skilled in the art. In certain embodiments, a siRNA agent described herein comprising one or more modified nucleotides exhibits increased stability relative to the same siRNA agent without those one or more modified nucleotides, as assessed using a method known to one skilled in the art, or described herein (e.g., in Section 6.8, infra, or Section 7, infra).

In one embodiment, provided herein is a siRNA agent comprising a sense strand and an antisense strand forming a double stranded region, wherein the sense strand comprises the nucleotide sequence of any one of the sense strand nucleotide sequences in Table 2. In one embodiment, provided herein is a siRNA agent comprising a sense strand and an antisense strand forming a double stranded region, wherein the antisense strand comprises the nucleotide sequence of any one of the antisense strand nucleotide sequences in Table 2.

In one embodiment, provided herein is a siRNA agent comprising a sense strand and an antisense strand forming a double stranded region, wherein the antisense strand comprises the nucleotide sequence of the antisense strand nucleotide sequence in a row in Table 2, and wherein the sense strand comprises the nucleotide sequence of the sense strand nucleotide sequence in the same row in Table 2.

In certain embodiments, provided herein is a siRNA agent comprising a sense strand and an antisense strand forming a double stranded region, wherein the antisense strand comprises the nucleotide sequence of any one of the antisense strand nucleotide sequences in Table 2, and the sense strand comprises a nucleotide sequence partially complementary to the antisense strand. In some embodiments, provided herein is a siRNA agent comprising a sense strand and an antisense strand forming a double stranded region, wherein the antisense strand comprises the nucleotide sequence of any one of the antisense strand nucleotide sequences in Table 2, and the sense strand comprises a nucleotide sequence substantially complementary to the antisense strand. In certain embodiments, provided herein is a siRNA agent comprising a sense strand and an antisense strand forming a double stranded region, wherein the antisense strand comprises the nucleotide sequence of any one of the antisense strand nucleotide sequences in Table 2, and the sense strand comprises a nucleotide sequence fully complementary to the antisense strand.

In certain embodiments, provided herein is a siRNA agent comprising a sense strand and an antisense strand forming a double stranded region, wherein the sense strand comprises the nucleotide sequence of any one of the sense strand nucleotide sequences in Table 2, and the antisense strand comprises a nucleotide sequence partially complementary to the sense strand. In some embodiments, provided herein is a siRNA agent comprising a sense strand and an antisense strand forming a double stranded region, wherein the sense strand comprises the nucleotide sequence of any one of the sense strand nucleotide sequences in Table 2, and the antisense strand comprises a nucleotide sequence substantially complementary to the sense strand. In certain embodiments, provided herein is a siRNA agent comprising a sense strand and an antisense strand forming a double stranded region, wherein the sense strand comprises the nucleotide sequence of any one of the sense strand nucleotide sequences in Table 2, and the antisense strand comprises a nucleotide sequence fully complementary to the sense strand.

In one embodiment, provided herein is a siRNA agent comprising a sense strand and an antisense strand forming a double stranded region, wherein the sense strand comprises the nucleotide sequence of any one of the sense strand nucleotide sequences in Table 3. In one embodiment, provided herein is a siRNA agent comprising a sense strand and an antisense strand forming a double stranded region, wherein the antisense strand comprises the nucleotide sequence of any one of the antisense strand nucleotide sequences in Table 3. In one embodiment, provided herein is a siRNA agent comprising a sense strand and an antisense strand forming a double stranded region, wherein the antisense strand comprises the nucleotide sequence of the antisense strand nucleotide sequence in a row in Table 3, and the sense strand comprises the nucleotide sequence of the sense strand nucleotide sequence in the same row in Table 3.

In certain embodiments, provided herein is a siRNA agent comprising a sense strand and an antisense strand forming a double stranded region, wherein the antisense strand comprises the nucleotide sequence of any one of the antisense strand nucleotide sequences in Table 3, and the sense strand comprises a nucleotide sequence partially complementary to the antisense strand. In some embodiments, provided herein is a siRNA agent comprising a sense strand and an antisense strand forming a double stranded region, wherein the antisense strand comprises the nucleotide sequence of any one of the antisense strand nucleotide sequences in Table 3, and the sense strand comprises a nucleotide sequence substantially complementary to the antisense strand. In certain embodiments, provided herein is a siRNA agent comprising a sense strand and an antisense strand forming a double stranded region, wherein the antisense strand comprises the nucleotide sequence of any one of the antisense strand nucleotide sequences in Table 3, and the sense strand comprises a nucleotide sequence fully complementary to the antisense strand.

In certain embodiments, provided herein is a siRNA agent comprising a sense strand and an antisense strand forming a double stranded region, wherein the sense strand comprises the nucleotide sequence of any one of the sense strand nucleotide sequences in Table 3, and the antisense strand comprises a nucleotide sequence partially complementary to the sense strand. In some embodiments, provided herein is a siRNA agent comprising a sense strand and an antisense strand forming a double stranded region, wherein the sense strand comprises the nucleotide sequence of any one of the sense strand nucleotide sequences in Table 3, and the antisense strand comprises a nucleotide sequence substantially complementary to the sense strand. In certain embodiments, provided herein is a siRNA agent comprising a sense strand and an antisense strand forming a double stranded region, wherein the sense strand comprises the nucleotide sequence of any one of the sense strand nucleotide sequences in Table 3, and the antisense strand comprises a nucleotide sequence fully complementary to the sense strand.

In some embodiments, a siRNA agent described herein is conjugated to one or more non-nucleotide groups including, but not limited to a targeting group, linking group, delivery polymer, or a delivery vehicle. See, e.g., for additional information regarding Section 6.3, infra, for siRNA agents conjugated to one or more non-nucleotide groups. In a specific embodiment, the one or more non-nucleotide groups is one or more of the ones described herein (e.g., in Section 6.3, infra, or Section 7, infra).

In specific embodiments, the antisense strand of a siRNA agent described herein is conjugated directly or indirectly to one or more lipophilic moieties. See, e.g., S. Winiwarter, M. Ridderstram, A. L. Ungell, T. B. Andersson, I. Zamora, Use of Molecular Descriptors for Absorption, Distribution, Metabolism, and Excretion Predictions. Comprehensive Medicinal Chemistry II, Volume 5, 2007, pages 531-554 regarding lipophilicity. In specific embodiments, the sense strand of a siRNA agent described herein is conjugated directly or indirectly to one or more lipophilic moieties. In specific embodiments, the antisense strand and sense strand of a siRNA agent described herein are each conjugated directly or indirectly to one or more lipophilic moieties. The one or more lipophilic moieties may be conjugated to one or more terminal positions of the siRNA agent. For example, the lipophilic moieties may be conjugated to the 5′ end and/or 3′ end of one or both strands of the siRNA agent. Alternatively, or in addition, the one or more lipophilic moieties may be conjugated to one or more internal positions of the double stranded region of the siRNA agent. In certain embodiments, the lipophilic moieties are conjugated to one or more terminal positions of the siRNA agent and one or more internal positions of the siRNA agent. In some embodiments, the one or more lipophilic moieties are conjugated via a linker or a carrier. In a specific embodiment, a linker is one described herein (e.g., in Section 6.3, infra). For example, the linker may comprise one of the following structures:

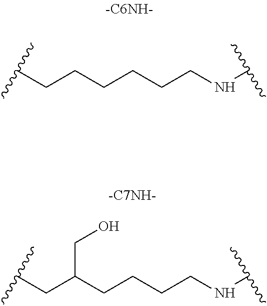 [SMILES: *CCCCCCN* ; *CC(CO)CCCCN*]

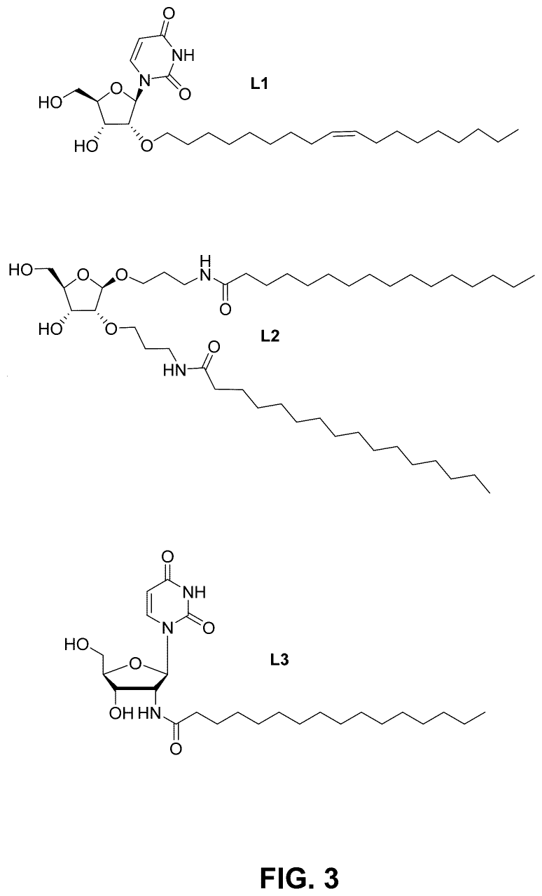

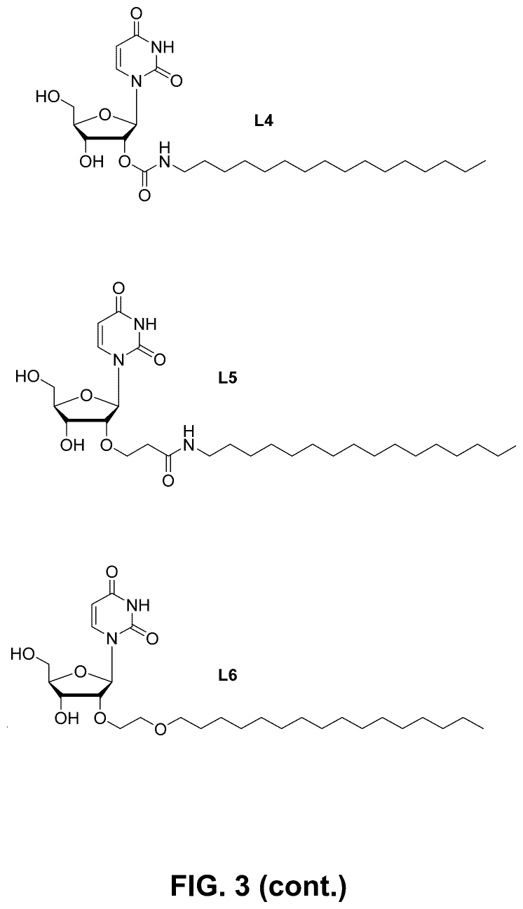

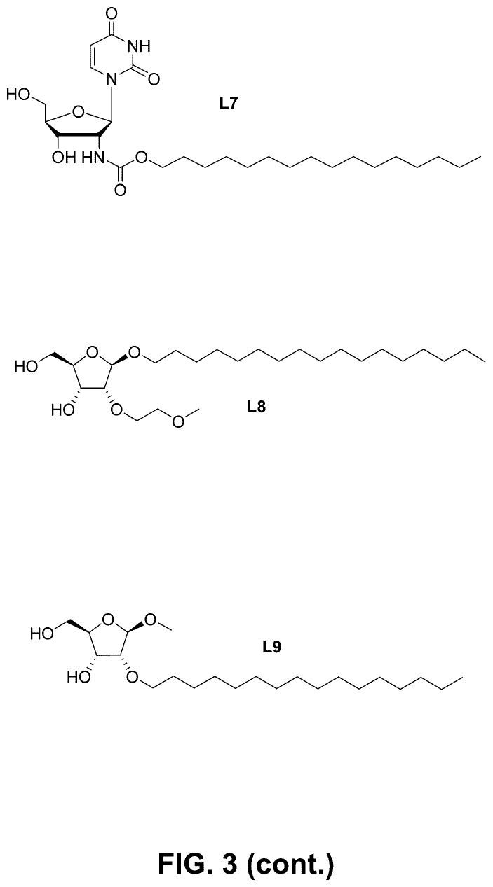

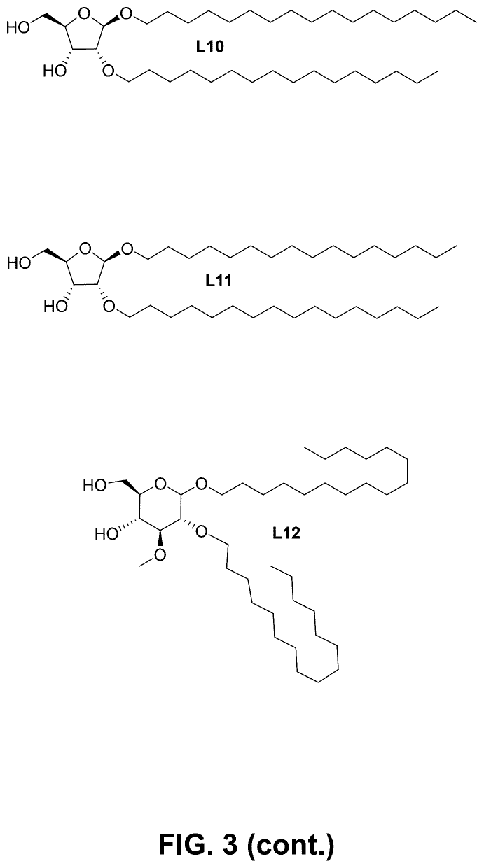

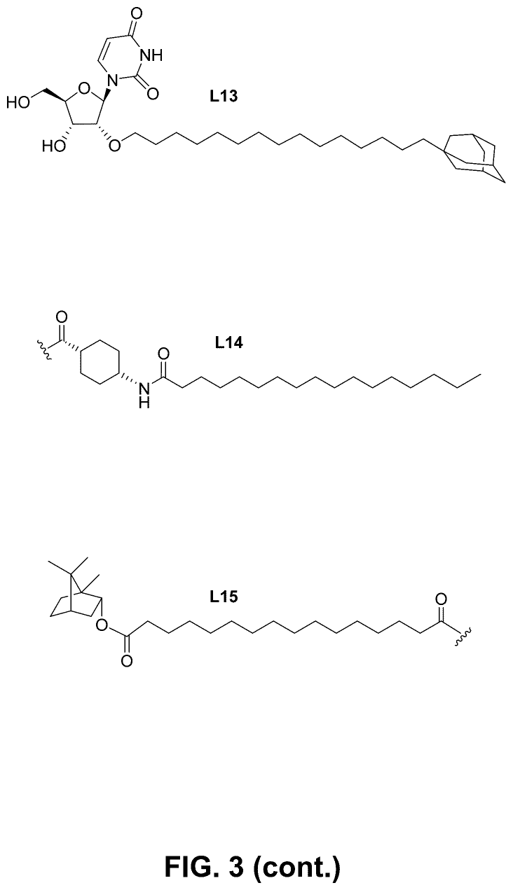

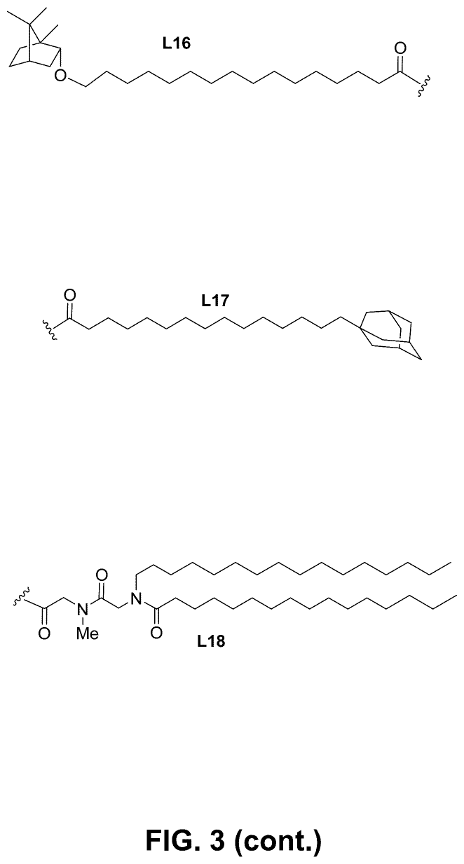

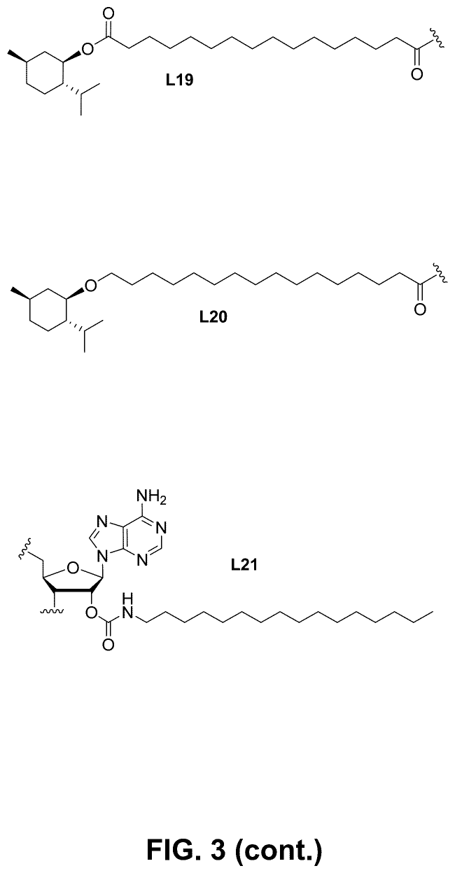

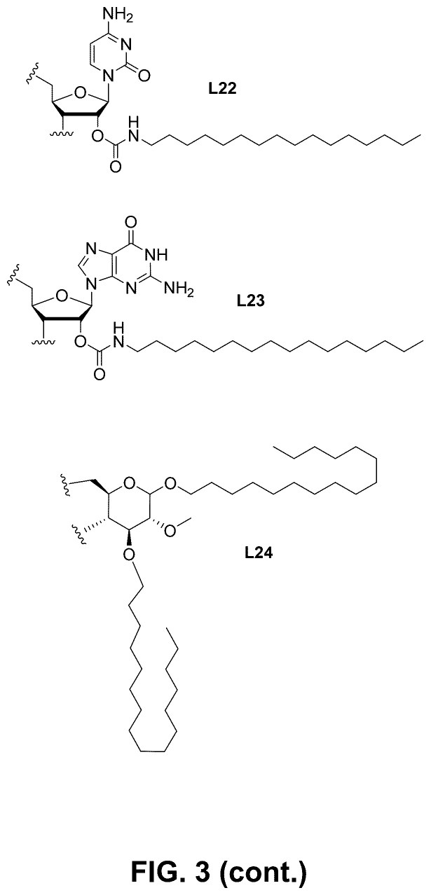

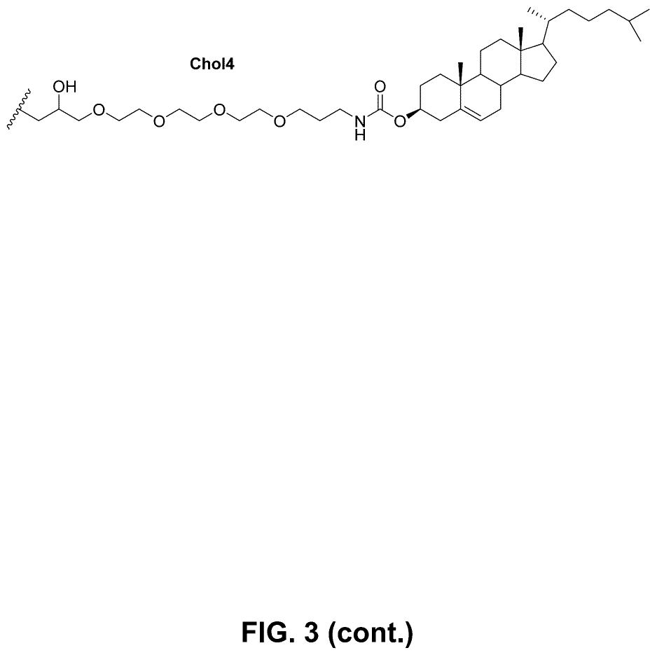

In specific embodiments, a lipophilic moiety comprises a lipophilic moiety described herein (e.g., in Section 6.3, infra, Section 6.4, infra, or Section 7, infra). In specific embodiments, a lipophilic moiety is cholesterol. In specific embodiments, a lipophilic moiety is selected from one or more of the following: L1, L2, L3, L4, L5, L6, L7, L8, L9, L10, L11, L12, L13, L14, L15, L16, L17, L18, L19, L20, L20, L21, L22, L23, and/or L24 (see FIG. 3). In specific embodiments, a lipophilic moiety comprises cholesterol-triethylene glycol; 1-((2R,3R,4R,5R)-4-hydroxy-5-(hydroxymethyl)-3-(((Z)-octadec-9-en-1-yl)oxy)tetrahydrofuran-2-yl)pyrimidine-2,4(1H,3H)-dione; N,N′-((((2R,3R,4R,5R)-4-hydroxy-5-(hydroxymethyl)tetrahydrofuran-2,3-diyl)bis(oxy))bis(propane-3,1-diyl))dipalmitamide; N-((2R,3R,4S,5R)-2-(2,4-dioxo-3,4-dihydropyrimidin-1(2H)-yl)-4-hydroxy-5-(hydroxymethyl)tetrahydrofuran-3-yl)palmitamide; (2R,3R,4R,5R)-2-(2,4-dioxo-3,4-dihydropyrimidin-1(2H)-yl)-4-hydroxy-5-(hydroxymethyl)tetrahydrofuran-3-yl hexadecylcarbamate; 3-(((2R,3R,4R,5R)-2-(2,4-dioxo-3,4-dihydropyrimidin-1(2H)-yl)-4-hydroxy-5-(hydroxymethyl)tetrahydrofuran-3-yl)oxy)-N-hexadecylpropanamide; 1-((2R,3R,4R,5R)-3-(2-(hexadecyloxy)ethoxy)-4-hydroxy-5-(hydroxymethyl)tetrahydrofuran-2-yl)pyrimidine-2,4(1H,3H)-dione; Hexadecyl ((2R,3R,4S,5R)-2-(2,4-dioxo-3,4-dihydropyrimidin-1(2H)-yl)-4-hydroxy-5-(hydroxymethyl)tetrahydrofuran-3-yl)carbamate; (2R,3R,4R,5R)-5-(heptadecyloxy)-2-(hydroxymethyl)-4-(2-methoxyethoxy)tetrahydrofuran-3-ol; (2R,3R,4R,5R)-4-(hexadecyloxy)-2-(hydroxymethyl)-5-methoxytetrahydrofuran-3-ol; (2R,3R,4R,5R)-5-(heptadecyloxy)-4-(hexadecyloxy)-2-(hydroxymethyl)tetrahydrofuran-3-ol; (2R,3R,4R,5R)-4,5-bis(hexadecyloxy)-2-(hydroxymethyl)tetrahydrofuran-3-ol; (2R,3R,4S,5R)-5,6-bis(hexadecyloxy)-2-(hydroxymethyl)-4-methoxytetrahydro-2H-pyran-3-ol; 1-((2R,3R,4R,5R)-3-((15-((3r,5r,7r)-adamantan-1-yl)pentadecyl)oxy)-4-hydroxy-5-(hydroxymethyl)tetrahydrofuran-2-yl)pyrimidine-2,4(1H,3H)-dione; (1s,4s)-4-heptadecanamidocyclohexane-1-carboxyl-; 16-oxo-16-(((1S,2R,4S)-1,7,7-trimethylbicyclo[2.2.1]heptan-2-yl)oxy)hexadecanoyl-; 16-(((1S,2R,4S)-1,7,7-trimethylbicyclo[2.2.1]heptan-2-yl)oxy)hexadecanoyl-; 15-((3r,5r,7r)-adamantan-1-yl)pentadecanoyl-; 2-(2-(N-hexadecylpalmitamido)-N-methylacetamido)ethanoyl-; 16-(((1R,2S,5R)-2-isopropyl-5-methylcyclohexyl)oxy)-16-oxohexadecanonyl-; 16-(((1R,2S,5R)-2-isopropyl-5-methylcyclohexyl)oxy)hexadecananonyl-; (2R,3R,4R,5R)-2-(6-amino-9H-purin-9-yl)-4-hydroxy-5-(hydroxymethyl)tetrahydrofuran-3-yl hexadecylcarbamate; (2R,3R,4R,5R)-2-(4-amino-2-oxopyrimidin-1(2H)-yl)-4-hydroxy-5-(hydroxymethyl)tetrahydrofuran-3-yl hexadecylcarbamate; (2R,3R,4R,5R)-2-(2-amino-6-oxo-1,6-dihydro-9H-purin-9-yl)-4-hydroxy-5-(hydroxymethyl)tetrahydrofuran-3-yl hexadecylcarbamate; or (2R,3R,4S,5R,6R)-4,6-bis(hexadecyloxy)-2-(hydroxymethyl)-5-methoxytetrahydro-2H-pyran-3-ol. In a specific embodiment, a lipophilic moiety, such as described in Section 7, is conjugated to the sense strand of a siRNA agent in the manner described in Section 7.

In one embodiment, provided herein is a siRNA agent comprising a sense strand and an antisense strand forming a double stranded region, wherein the sense strand comprises the nucleotide sequence of any one of the sense strand nucleotide sequences in Table 9. In one embodiment, provided herein is a siRNA agent comprising a sense strand and an antisense strand forming a double stranded region, wherein the antisense strand comprises the nucleotide sequence of any one of the antisense strand nucleotide sequences in Table 9. In one embodiment, provided herein is a siRNA agent comprising a sense strand and an antisense strand forming a double stranded region, wherein the antisense strand comprises the nucleotide sequence of the antisense strand nucleotide sequence in a row in Table 9, and the sense strand comprises the nucleotide sequence of the sense strand nucleotide sequence in the same row in Table 9. In one embodiment, provided herein is a siRNA agent comprising a sense strand and an antisense strand forming a double stranded region, wherein the sense strand comprises the nucleotide sequence of any one of the sense strand nucleotide sequences in Table 9, and wherein the sense strand is conjugated to cholesterol as indicated in Table 9 conjugated to the sense strand of the siRNA agent. In one embodiment, provided herein is a siRNA agent comprising a sense strand and an antisense strand forming a double stranded region, wherein the antisense strand comprises the nucleotide sequence of the antisense strand nucleotide sequence in a row in Table 9, and the sense strand comprises the nucleotide sequence of the sense strand nucleotide sequence in the same row in Table 9, and wherein the sense strand is conjugated to cholesterol as indicated in Table 9 conjugated to the sense strand of the siRNA agent.

In certain embodiments, provided herein is a siRNA agent comprising a sense strand and an antisense strand forming a double stranded region, wherein the antisense strand comprises the nucleotide sequence of any one of the antisense strand nucleotide sequences in Table 9, and the sense strand comprises a nucleotide sequence partially complementary to the antisense strand. In some embodiments, provided herein is a siRNA agent comprising a sense strand and an antisense strand forming a double stranded region, wherein the antisense strand comprises the nucleotide sequence of any one of the antisense strand nucleotide sequences in Table 9, and the sense strand comprises a nucleotide sequence substantially complementary to the antisense strand. In certain embodiments, provided herein is a siRNA agent comprising a sense strand and an antisense strand forming a double stranded region, wherein the antisense strand comprises the nucleotide sequence of any one of the antisense strand nucleotide sequences in Table 9, and the sense strand comprises a nucleotide sequence fully complementary to the antisense strand.

In certain embodiments, provided herein is a siRNA agent comprising a sense strand and an antisense strand forming a double stranded region, wherein the antisense strand comprises the nucleotide sequence of any one of the antisense strand nucleotide sequences in Table 9, and the sense strand comprises a nucleotide sequence partially complementary to the antisense strand, and wherein the sense strand of the siRNA agent is conjugated to cholesterol as indicated in Table 9, or another lipid moiety, such as, e.g., described in FIG. 3. In some embodiments, provided herein is a siRNA agent comprising a sense strand and an antisense strand forming a double stranded region, wherein the antisense strand comprises the nucleotide sequence of any one of the antisense strand nucleotide sequences in Table 9, and the sense strand comprises a nucleotide sequence substantially complementary to the antisense strand, and wherein the sense strand of the siRNA agent is conjugated to cholesterol as indicated in Table 9, or another lipid moiety, such as, e.g., described in FIG. 3. In certain embodiments, provided herein is a siRNA agent comprising a sense strand and an antisense strand forming a double stranded region, wherein the antisense strand comprises the nucleotide sequence of any one of the antisense strand nucleotide sequences in Table 9, and the sense strand comprises a nucleotide sequence fully complementary to the antisense strand, and wherein the sense strand of the siRNA agent is conjugated to cholesterol as indicated in Table 9, or another lipid moiety, such as, e.g., described in FIG. 3.

In certain embodiments, provided herein is a siRNA agent comprising a sense strand and an antisense strand forming a double stranded region, wherein the sense strand comprises the nucleotide sequence of any one of the sense strand nucleotide sequences in Table 9, and the antisense strand comprises a nucleotide sequence partially complementary to the sense strand. In some embodiments, provided herein is a siRNA agent comprising a sense strand and an antisense strand forming a double stranded region, wherein the sense strand comprises the nucleotide sequence of any one of the sense strand nucleotide sequences in Table 9, and the antisense strand comprises a nucleotide sequence substantially complementary to the sense strand. In certain embodiments, provided herein is a siRNA agent comprising a sense strand and an antisense strand forming a double stranded region, wherein the sense strand comprises the nucleotide sequence of any one of the sense strand nucleotide sequences in Table 9, and the antisense strand comprises a nucleotide sequence fully complementary to the sense strand.

In certain embodiments, provided herein is a siRNA agent comprising a sense strand and an antisense strand forming a double stranded region, wherein the sense strand comprises the nucleotide sequence of any one of the sense strand nucleotide sequences in Table 9, wherein the sense strand is conjugated to cholesterol as indicated in Table 9 conjugated to the sense strand of the siRNA agent, and the antisense strand comprises a nucleotide sequence partially complementary to the sense strand. In some embodiments, provided herein is a siRNA agent comprising a sense strand and an antisense strand forming a double stranded region, wherein the sense strand comprises the nucleotide sequence of any one of the sense strand nucleotide sequences in Table 9, wherein the sense strand is conjugated to cholesterol as indicated in Table 9, and the antisense strand comprises a nucleotide sequence substantially complementary to the sense strand. In certain embodiments, provided herein is a siRNA agent comprising a sense strand and an antisense strand forming a double stranded region, wherein the sense strand comprises the nucleotide sequence of any one of the sense strand nucleotide sequences in Table 9, wherein the sense strand is conjugated to cholesterol as indicated in Table 9, and the antisense strand comprises a nucleotide sequence fully complementary to the sense strand.

In one embodiment, provided herein is a siRNA agent comprising a sense strand and an antisense strand forming a double stranded region, wherein the sense strand comprises the nucleotide sequence of any one of the sense strand nucleotide sequences in Table 4, and wherein the sense strand is conjugated to cholesterol or another lipid as indicated in the same row of Table 4. In one embodiment, provided herein is a siRNA agent comprising a sense strand and an antisense strand forming a double stranded region, wherein the antisense strand comprises the nucleotide sequence of any one of the antisense strand nucleotide sequences in Table 4, and wherein the sense strand is conjugated to cholesterol or another lipid (e.g., a lipid in FIG. 3). In one embodiment, provided herein is a siRNA agent comprising a sense strand and an antisense strand forming a double stranded region, wherein the antisense strand comprises the nucleotide sequence of the antisense strand nucleotide sequence in a row in Table 4, and the sense strand comprises the nucleotide sequence of the sense strand nucleotide sequence in the same row in Table 4, wherein the sense strand is conjugated to cholesterol or another lipid as indicated in the same row of Table 4.

In certain embodiments, provided herein is a siRNA agent comprising a sense strand and an antisense strand forming a double stranded region, wherein the antisense strand comprises the nucleotide sequence of any one of the antisense strand nucleotide sequences in Table 4, and the sense strand comprises a nucleotide sequence partially complementary to the antisense strand, and wherein the sense strand is conjugated to cholesterol or another lipid (e.g., a lipid in FIG. 3). In some embodiments, provided herein is a siRNA agent comprising a sense strand and an antisense strand forming a double stranded region, wherein the antisense strand comprises the nucleotide sequence of any one of the antisense strand nucleotide sequences in Table 4, and the sense strand comprises a nucleotide sequence substantially complementary to the antisense strand, and wherein the sense strand is conjugated to cholesterol or another lipid (e.g., a lipid in FIG. 3). In certain embodiments, provided herein is a siRNA agent comprising a sense strand and an antisense strand forming a double stranded region, wherein the antisense strand comprises the nucleotide sequence of any one of the antisense strand nucleotide sequences in Table 4, and the sense strand comprises a nucleotide sequence fully complementary to the antisense strand, and wherein the sense strand is conjugated to cholesterol or another lipid (e.g., a lipid in FIG. 3).

In one embodiment, provided herein is a siRNA agent comprising a sense strand and an antisense strand forming a double stranded region, wherein the sense strand comprises the nucleotide sequence of any one of the sense strand nucleotide sequences in Table 5. In another embodiment, provided herein is a siRNA agent comprising a sense strand and an antisense strand forming a double stranded region, wherein the sense strand comprises the nucleotide sequence of any one of the sense strand nucleotide sequences in Table 5, wherein the sense strand is conjugated to a lipid moiety as indicated in the same row of Table 5.

In one embodiment, provided herein is a siRNA agent comprising a sense strand and an antisense strand forming a double stranded region, wherein the antisense strand comprises the nucleotide sequence of any one of the antisense strand nucleotide sequences in Table 5. In another embodiment, provided herein is a siRNA agent comprising a sense strand and an antisense strand forming a double stranded region, wherein the antisense strand comprises the nucleotide sequence of any one of the antisense strand nucleotide sequences in Table 5, and wherein the sense strand is conjugated to a lipophilic moiety described herein.

In one embodiment, provided herein is a siRNA agent comprising a sense strand and an antisense strand forming a double stranded region, wherein the antisense strand comprises the nucleotide sequence of the antisense strand nucleotide sequence in a row in Table 5, and the sense strand comprises the nucleotide sequence of the sense strand nucleotide sequence in the same row in Table 5. In another embodiment, provided herein is a siRNA agent comprising a sense strand and an antisense strand forming a double stranded region, wherein the antisense strand comprises the nucleotide sequence of the antisense strand nucleotide sequence in a row in Table 5, and the sense strand comprises the nucleotide sequence of the sense strand nucleotide sequence in the same row in Table 5, and wherein the sense strand is conjugated to a lipid moiety as indicated in the same row of Table 5.

In certain embodiments, provided herein is a siRNA agent comprising a sense strand and an antisense strand forming a double stranded region, wherein the antisense strand comprises the nucleotide sequence of any one of the antisense strand nucleotide sequences in Table 5, and the sense strand comprises a nucleotide sequence partially complementary to the antisense strand. In certain embodiments, provided herein is a siRNA agent comprising a sense strand and an antisense strand forming a double stranded region, wherein the antisense strand comprises the nucleotide sequence of any one of the antisense strand nucleotide sequences in Table 5, and the sense strand comprises a nucleotide sequence partially complementary to the antisense strand, and wherein the sense strand is conjugated to a lipophilic moiety described herein. In some embodiments, provided herein is a siRNA agent comprising a sense strand and an antisense strand forming a double stranded region, wherein the antisense strand comprises the nucleotide sequence of any one of the antisense strand nucleotide sequences in Table 5, and the sense strand comprises a nucleotide sequence substantially complementary to the antisense strand. In some embodiments, provided herein is a siRNA agent comprising a sense strand and an antisense strand forming a double stranded region, wherein the antisense strand comprises the nucleotide sequence of any one of the antisense strand nucleotide sequences in Table 5, and the sense strand comprises a nucleotide sequence substantially complementary to the antisense strand, and wherein the sense strand is conjugated to a lipophilic moiety described herein. In certain embodiments, provided herein is a siRNA agent comprising a sense strand and an antisense strand forming a double stranded region, wherein the antisense strand comprises the nucleotide sequence of any one of the antisense strand nucleotide sequences in Table 5, and the sense strand comprises a nucleotide sequence fully complementary to the antisense strand. In certain embodiments, provided herein is a siRNA agent comprising a sense strand and an antisense strand forming a double stranded region, wherein the antisense strand comprises the nucleotide sequence of any one of the antisense strand nucleotide sequences in Table 5, and the sense strand comprises a nucleotide sequence fully complementary to the antisense strand, and wherein the sense strand is conjugated to a lipophilic moiety described herein.

In certain embodiments, provided herein is a siRNA agent comprising a sense strand and an antisense strand forming a double stranded region, wherein the sense strand comprises the nucleotide sequence of any one of the sense strand nucleotide sequences in Table 5, and the antisense strand comprises a nucleotide sequence partially complementary to the sense strand. In certain embodiments, provided herein is a siRNA agent comprising a sense strand and an antisense strand forming a double stranded region, wherein the sense strand comprises the nucleotide sequence of any one of the sense strand nucleotide sequences in Table 5, wherein the sense strand is conjugated to a lipid moiety as indicated in the same row of Table 5, and the antisense strand comprises a nucleotide sequence partially complementary to the sense strand. In some embodiments, provided herein is a siRNA agent comprising a sense strand and an antisense strand forming a double stranded region, wherein the sense strand comprises the nucleotide sequence of any one of the sense strand nucleotide sequences in Table 5, and the antisense strand comprises a nucleotide sequence substantially complementary to the sense strand. In some embodiments, provided herein is a siRNA agent comprising a sense strand and an antisense strand forming a double stranded region, wherein the sense strand comprises the nucleotide sequence of any one of the sense strand nucleotide sequences in Table 5, wherein the sense strand is conjugated to a lipid moiety as indicated in the same row of Table 5, and the antisense strand comprises a nucleotide sequence substantially complementary to the sense strand. In certain embodiments, provided herein is a siRNA agent comprising a sense strand and an antisense strand forming a double stranded region, wherein the sense strand comprises the nucleotide sequence of any one of the sense strand nucleotide sequences in Table 5, and the antisense strand comprises a nucleotide sequence fully complementary to the sense strand. In certain embodiments, provided herein is a siRNA agent comprising a sense strand and an antisense strand forming a double stranded region, wherein the sense strand comprises the nucleotide sequence of any one of the sense strand nucleotide sequences in Table 5, wherein the sense strand is conjugated to a lipid moiety as indicated in the same row of Table 5, and the antisense strand comprises a nucleotide sequence fully complementary to the sense strand.

In one embodiment, provided herein is a siRNA agent comprising a sense strand and an antisense strand forming a double stranded region, wherein the sense strand comprises the nucleotide sequence of any one of the sense strand nucleotide sequences in Table 33. In another embodiment, provided herein is a siRNA agent comprising a sense strand and an antisense strand forming a double stranded region, wherein the sense strand comprises the nucleotide sequence of any one of the sense strand nucleotide sequences in Table 33, wherein the sense strand is conjugated to a lipid moiety as indicated in the same row of Table 33.

In one embodiment, provided herein is a siRNA agent comprising a sense strand and an antisense strand forming a double stranded region, wherein the antisense strand comprises the nucleotide sequence of any one of the antisense strand nucleotide sequences in Table 33. In another embodiment, provided herein is a siRNA agent comprising a sense strand and an antisense strand forming a double stranded region, wherein the antisense strand comprises the nucleotide sequence of any one of the antisense strand nucleotide sequences in Table 33, and wherein the sense strand is conjugated to a lipophilic moiety described herein.

In one embodiment, provided herein is a siRNA agent comprising a sense strand and an antisense strand forming a double stranded region, wherein the antisense strand comprises the nucleotide sequence of the antisense strand nucleotide sequence in a row in Table 33, and the sense strand comprises the nucleotide sequence of the sense strand nucleotide sequence in the same row in Table 33. In another embodiment, provided herein is a siRNA agent comprising a sense strand and an antisense strand forming a double stranded region, wherein the antisense strand comprises the nucleotide sequence of the antisense strand nucleotide sequence in a row in Table 33, and the sense strand comprises the nucleotide sequence of the sense strand nucleotide sequence in the same row in Table 33, and wherein the sense strand is conjugated to a lipid moiety as indicated in the same row of Table 33.

In certain embodiments, provided herein is a siRNA agent comprising a sense strand and an antisense strand forming a double stranded region, wherein the antisense strand comprises the nucleotide sequence of any one of the antisense strand nucleotide sequences in Table 33, and the sense strand comprises a nucleotide sequence partially complementary to the antisense strand. In certain embodiments, provided herein is a siRNA agent comprising a sense strand and an antisense strand forming a double stranded region, wherein the antisense strand comprises the nucleotide sequence of any one of the antisense strand nucleotide sequences in Table 33, and the sense strand comprises a nucleotide sequence partially complementary to the antisense strand, and wherein the sense strand is conjugated to a lipophilic moiety described herein. In some embodiments, provided herein is a siRNA agent comprising a sense strand and an antisense strand forming a double stranded region, wherein the antisense strand comprises the nucleotide sequence of any one of the antisense strand nucleotide sequences in Table 33, and the sense strand comprises a nucleotide sequence substantially complementary to the antisense strand. In some embodiments, provided herein is a siRNA agent comprising a sense strand and an antisense strand forming a double stranded region, wherein the antisense strand comprises the nucleotide sequence of any one of the antisense strand nucleotide sequences in Table 33, and the sense strand comprises a nucleotide sequence substantially complementary to the antisense strand, and wherein the sense strand is conjugated to a lipophilic moiety described herein. In certain embodiments, provided herein is a siRNA agent comprising a sense strand and an antisense strand forming a double stranded region, wherein the antisense strand comprises the nucleotide sequence of any one of the antisense strand nucleotide sequences in Table 33, and the sense strand comprises a nucleotide sequence fully complementary to the antisense strand. In certain embodiments, provided herein is a siRNA agent comprising a sense strand and an antisense strand forming a double stranded region, wherein the antisense strand comprises the nucleotide sequence of any one of the antisense strand nucleotide sequences in Table 33, and the sense strand comprises a nucleotide sequence fully complementary to the antisense strand, and wherein the sense strand is conjugated to a lipophilic moiety described herein.

In certain embodiments, provided herein is a siRNA agent comprising a sense strand and an antisense strand forming a double stranded region, wherein the sense strand comprises the nucleotide sequence of any one of the sense strand nucleotide sequences in Table 33, and the antisense strand comprises a nucleotide sequence partially complementary to the sense strand. In certain embodiments, provided herein is a siRNA agent comprising a sense strand and an antisense strand forming a double stranded region, wherein the sense strand comprises the nucleotide sequence of any one of the sense strand nucleotide sequences in Table 33, wherein the sense strand is conjugated to a lipid moiety as indicated in the same row of Table 33, and the antisense strand comprises a nucleotide sequence partially complementary to the sense strand. In some embodiments, provided herein is a siRNA agent comprising a sense strand and an antisense strand forming a double stranded region, wherein the sense strand comprises the nucleotide sequence of any one of the sense strand nucleotide sequences in Table 33, and the antisense strand comprises a nucleotide sequence substantially complementary to the sense strand. In some embodiments, provided herein is a siRNA agent comprising a sense strand and an antisense strand forming a double stranded region, wherein the sense strand comprises the nucleotide sequence of any one of the sense strand nucleotide sequences in Table 33, wherein the sense strand is conjugated to a lipid moiety as indicated in the same row of Table 33, and the antisense strand comprises a nucleotide sequence substantially complementary to the sense strand. In certain embodiments, provided herein is a siRNA agent comprising a sense strand and an antisense strand forming a double stranded region, wherein the sense strand comprises the nucleotide sequence of any one of the sense strand nucleotide sequences in Table 33, and the antisense strand comprises a nucleotide sequence fully complementary to the sense strand. In certain embodiments, provided herein is a siRNA agent comprising a sense strand and an antisense strand forming a double stranded region, wherein the sense strand comprises the nucleotide sequence of any one of the sense strand nucleotide sequences in Table 33, wherein the sense strand is conjugated to a lipid moiety as indicated in the same row of Table 33, and the antisense strand comprises a nucleotide sequence fully complementary to the sense strand.

In specific embodiments, a siRNA agent described herein comprises the antisense strand and the sense strand of one named in Table 1, 2, 3, 4, 5, 9, or 33. In specific embodiments, a siRNA agent described herein comprises an antisense strand and a sense strand corresponding to the antisense strand and the sense strand of any one of UM25, UM28, UM96, UM165, or UM177 in Table 1. In specific embodiments, a siRNA agent described herein comprises the antisense strand and the sense strand of any one of UM25, UM28, UM96, UM165, or UM177 in Table 2. In specific embodiments, a siRNA agent described herein comprises the antisense strand and the sense strand of one named in Table 3, 4, 5, 9, or 33. In some embodiments, the sense strand of the siRNA agent is conjugated to a lipid moiety described herein (e.g., FIG. 3). In specific embodiments, a siRNA agent is one described in Section 7, infra.

In some embodiments, a siRNA agent described herein further comprises a non-nucleotide group, such as a ligand (e.g., a targeting ligand). The non-nucleotide group, such as a ligand (e.g., a targeting ligand) may be conjugated directly or directly to the antisense strand or sense strand of the siRNA agent. The non-nucleotide group, such as a ligand (e.g., a targeting ligand) replace one or more nucleotides in an internal position of the double stranded region of a siRNA agent described herein. In certain embodiments, the non-nucleotide group, such as a ligand (e.g., a targeting ligand) is conjugated to a strand of the siRNA agent via a linker or carrier (e.g., a delivery vehicle). In a specific embodiment, the non-nucleotide group, such as a ligand (e.g., a targeting ligand) is one described in Section 6.3, infra.

TABLE 3
Modified Nucleotide Sense and Antisense Sequences (5′ to 3′)

| | | SEQ ID | | SEQ ID |
|---|---|---|---|---|
| Name | Sense strand | NO | Antisense strand | NO |
| M25 | mApsmGpsfUmCfUmAfCmAfAm | 357 | (VPmU)psfUpsmCfAmAfCmUfGm | 358 |
| | AfCmCfAmGfUmUfGmAfApsmUp | | GfUmUfUmGfUmAfGmAfCmUps | |
| | smU | | mUpsmU | |
| M25_V | mApsmGpsfUmCmUmAfCfAfAm | 359 | (VPmU)psfUpsmCmAmAfCmUm | 360 |
| ar1 | AmCfCmAmGmUmUfGmAmA | | GmGmUmUmUmGfUmAfGmAm | |
| | | | CmUpsmApsmU | |
| M25 | (InvAb)psmApsmGmUmCmUmAf | 361 | (VPmU)psfUpsmCpsfAmAfCmUf | 362 |
| Var2 | CfAfAmAmCmCmAmGmUmUmG | | GmGfUmUfUmGfUmAfGmAfCm | |
| | mApsmAps(InvAb) | | UmApsmU | |
| M25_V | mApsmGpsmUmCfUmAfCfAfAm | 363 | (VPmU)psfUpsmCpsmAmAfCmUf | 364 |
| ar3 | AmCmCmAmGmUmUmGpsmAp | | GfGmUmUmUmGfUmAfGmAmC | |
| | smA | | mUpsmApsmU | |
| M25_V | mApsmUpsmAmGmUmCfUmAfC | 365 | (VPmU)psfUpsmCmAmAfCmUfGf | 366 |
| ar4 | fAfAmAmCmCmAmGmUmUmGp | | GmUmUmUmGfUmAfGmAmCm | |
| | smApsmA | | UmAmUpsmUpsmU | |
| M25_V | mUpsmApsmGmUmCmUmAmCf | 367 | (VPmU)psfUpsmCmAmAmCfUm | 368 |
| ar5 | AmAfAfCfCfAmGmUmUmGmAm | | GmGmUmUfUmGfUmAmGmAm | |
| | Aps(InvAb) | | CmUmAmUpsmUpsmU | |
| M25_V | (InvAb)psmApsmGmUmCmUmAf | 369 | (VPmU)psfUpsmCpsmAmAfCmU | 370 |
| ar6 | CfAfAmAmCmCmAmGmUmUmG | | mGmGmUmUmUmGfUmAfGmA | |
| | mAmCpsmCpsmA | | mCmUmApsmU | |
| M25_V | (InvAb)psmApsmUmAmGmUmC | 371 | (VPmU)psfUpsmCpsfAmAfCmUf | 372 |
| ar7 | mUmAfCfAfAmAmCmCmAmGm | | GmGfUmUfUmGfUmAfGmAfCm | |
| | UmUmGmApsmAps(InvAb) | | UmApsmU | |
| M25_V | (InvAb)psmApsmGmUmCmUmAf | 373 | (VPmU)psfUpsmCmAmAfCmUfGf | 374 |
| ar8 | CfAfAmAmCmCmAmGmUmUmG | | GmUmUmUmGfUmAfGmAmCm | |
| | mApsmAps(InvAb) | | UmAmUpsmUpsmU | |
| M25_V | mApsmUpsmAmGmUmCfUmAfC | 375 | (VPmU)psfUpsmCpsfAmAfCmUf | 376 |
| ar9 | fAfAmAmCmCmAmGmUmUmGp | | GmGfUmUfUmGfUmAfGmAfCm | |
| | smApsmA | | UmApsmU | |
| M25_V | (InvAb)psmApsmGmUmCmUmAf | 377 | (VPmU)psfUpsmCpsmAmAfCmUf | 378 |
| ar10 | CfAfAmAmCmCmAmGmUmUmG | | GfGmUmUmUmGfUmAfGmAmC | |
| | mApsmAps(InvAb) | | mUpsmApsmU | |
| M25_V | mApsmGpsmUmCfUmAfCfAfAm | 379 | (VPmU)psfUpsmCpsfAmAfCmUf | 380 |
| ar11 | AmCmCmAmGmUmUmGpsmAp | | GmGfUmUfUmGfUmAfGmAfCm | |
| | smA | | UmApsmU | |
| M25_V | (InvAb)psmApsmGmUmCmUmAf | 381 | (VPmU)psfUpsmCmAmAmCfUm | 382 |
| ar12 | CfAfAmAmCmCmAmGmUmUmG | | GmGmUmUfUmGfUmAmGmAm | |
| | mApsmAps(InvAb) | | CmUmAmUpsmUpsmU | |
| M25_V | mUpsmApsmGmUmCmUmAmCf | 383 | (VPmU)psfUpsmCpsfAmAfCmUf | 384 |
| ar13 | AmAfAfCfCfAmGmUmUmGmAm | | GmGfUmUfUmGfUmAfGmAfCm | |
| | Aps(InvAb) | | UmApsmU | |
| M25_V | (InvAb)psmApsmUmAmGmUmC | 385 | (VPmU)psfUpsmCmAmAfCmUfGf | 386 |
| ar14 | mUmAfCfAfAmAmCmCmAmGm | | GmUmUmUmGfUmAfGmAmCm | |
| | UmUmGmApsmAps(InvAb) | | UmAmUpsmUpsmU | |
| M25_V | (InvAb)psmApsmUmAmGmUmC | 387 | (VPmU)psfUpsmCpsmAmAfCmUf | 388 |
| ar15 | mUmAfCfAfAmAmCmCmAmGm | | GfGmUmUmUmGfUmAfGmAmC | |
| | UmUmGmApsmAps(InvAb) | | mUpsmApsmU | |
| M25_V | (InvAb)psmApsmUmAmGmUmC | 389 | (VPmU)psfUpsmCmAmAmCfUm | 390 |
| ar16 | mUmAfCfAfAmAmCmCmAmGm | | GmGmUmUfUmGfUmAmGmAm | |
| | UmUmGmApsmAps(InvAb) | | CmUmAmUpsmUpsmU | |
| M25_V | mApsmUpsmAmGmUmCfUmAfC | 391 | (VPmU)psfUpsmCmAmAfCmUm | 392 |
| ar17 | fAfAmAmCmCmAmGmUmUmGp | | GfGmUmUmUmGfUmAfGmAmC | |
| | smApsmA | | mUmAmUpsmUpsmU | |
| M25_V | mApsmUpsmAmGmUmCfUmAfC | 393 | (VPmU)psfUpsmCmAmAfCmUfG | 394 |
| ar18 | fAfAmAmCmCmAmGmUmUmGp | | mGmUmUmUmGfUmAfGmAmC | |
| | smApsmA | | mUmAmUpsmUpsmU | |
| M25_V | mApsmUpsmAmGmUmCfUmAfC | 395 | (VPmU)psfUpsmCmAmAfCmUm | 396 |
| ar19 | fAfAmAmCmCmAmGmUmUmGp | | GmGmUmUmUmGfUmAfGmAm | |
| | smApsmA | | CmUmAmUpsmUpsmU | |
| M25_V | (InvAb)psmApsmUmAmGmUmC | 397 | (VPmU)psfUpsmCmAmAmC(UNA | 398 |
| ar20 | mUmAfCfAfAmAmCmCmAmGm | | U)mGmGmUmUmUmGfUmAfG | |
| | UmUmGmApsmAps(InvAb) | | mAmCmUpsmApsmU | |
| M25_V | (InvAb)psmApsmGmUmCmUmAf | 399 | (VPmU)psfUpsmCmAmAmC(UNA | 400 |
| ar21 | CfAfAmAmCmCmAmGmUmUmG | | U)mGmGmUmUmUmGfUmAfG | |
| | mApsmAps(InvAb) | | mAmCmUmAmUpsmUpsmU | |
| M25_V | (InvAb)psmApsmUmAmGmUmC | 401 | (VPmU)psfUpsmCmAmAfCmUfGf | 402 |
| ar22 | mUmAfCfAdAmAmCmCmAmGm | | GmUmUmUmGfUmAfGmAmCm | |
| | UmUmGmApsmAps(InvAb) | | UpsmApsmU | |
| M25_V | (InvAb)psmApsmUmAmGmUmC | 403 | (VPmU)psfUpsmCpsmAmAfCmUf | 404 |
| ar23 | mUmAfCfAfAmAmCmCmAmGm | | GfGmUmUmUmGfUmAfGmAmC | |
| | UmUmGmApsmAps(InvAb) | | mUmApsmU | |
| M25_V | mApsmGpsfUmCmUmAfCfAfAm | 405 | (VPmU)psfUpsmCpsmAmAfC(UN | 406 |
| ar24 | AmCfCmAmGmUmUfGpsmApsm | | A- | |
| | A | | U)mGmGmUmUmUmGfUmAfG | |
| | | | mAmCmUmApsmU | |
| M25_V | (InvAb)psmApsmGfUmCmUmAfC | 407 | (VPmU)psfUpsmCmAmAfC(UNA- | 408 |
| ar25 | fAfAmAmCfCmAmGmUmUfGps | | U)mGmGmUmUmUmGfUmAfG | |
| | mApsmA | | mAmCmUpsmApsmU | |
| M25_V | (InvAb)psmApsmGfUmCmUmAfC | 409 | (VPmU)psfUpsmCpsmAmAfC(UN | 410 |
| ar26 | fAfAmAmCfCmAmGmUmUfGps | | A- | |
| | mApsmA | | U)mGmGmUmUmUmGfUmAfG | |
| | | | mAmCmUmApsmU | |
| M25_V | (InvAb)psmApsmGmUmCmUmAf | 411 | (VPmU)psfUpsmCmAmAfCmUm | 412 |
| ar27 | CfAfAmAmCmCmAmGmUmUmG | | GfGmUmUmUmGfUmAfGmAmC | |
| | mApsmAps(InvAb) | | mUpsmApsmU | |
| M25_V | (InvAb)psmApsmUmAmGmUmC | 413 | (VPmU)psfUpsmCmAmAmC(UNA | 414 |
| ar28 | mUmAfCfAfAmAmCmCmAmGm | | - | |
| | UmUmGmApsmAps(InvAb) | | U)mGmGmUmUmUmGfUmAfG | |
| | | | mAmCmUpsmApsmU | |
| M25_V | (InvAb)psmApsmUmAmGmUmC | 415 | (VPmU)psfUpsmCmAmAfC(UNA- | 416 |
| ar29 | mUmAfCfAfAmAmCmCmAmGm | | U)mGmGmUmUmUmGfUmAfG | |
| | UmUmGmApsmAps(InvAb) | | mAmCmUpsmApsmU | |
| M25_V | (InvAb)psmApsmUmAmGmUmC | 417 | (VPmU)psfUpsmCmAmAmC(UNA | 418 |
| ar30 | mUmAfCfAfAmAmCmCmAmGm | | U)fGmGmUmUmUmGfUmAfGm | |
| | UmUmGmApsmAps(InvAb) | | AmCmUpsmApsmU | |
| M25_V | (InvAb)psmApsmUmAmGmUmC | 419 | (VPmU)psfUpsmCmAmAmC(UNA | 420 |
| ar31 | mUmAfCfAfAmAmCmCmAmGm | | U)mGfGmUmUmUmGfUmAfGm | |
| | UmUmGmApsmAps(InvAb) | | AmCmUpsmApsmU | |
| M25_V | (InvAb)psmApsmUmAmGmUmC | 421 | (VPmU)psfUpsmCmAmAfC(UNA- | 422 |
| ar32 | mUmAfCfAfAmAmCmCmAmGm | | U)mGfGmUmUmUmGfUmAfGm | |
| | UmUmGmApsmAps(InvAb) | | AmCmUpsmApsmU | |
| M25_V | (InvAb)psmApsmUmAmGmUmC | 423 | (VPmU)psfUpsmCmAmAmC(GNA | 424 |
| ar33 | mUmAfCfAfAmAmCmCmAmGm | | T)mGmGmUmUmUmGfUmAfGm | |
| | UmUmGmApsmAps(InvAb) | | AmCmUpsmApsmU | |
| M25_V | (InvAb)psmApsmUmAmGmUmC | 425 | (VPmU)psfUpsmCmAmAmCdTm | 426 |
| ar34 | mUmAfCfAfAmAmCmCmAmGm | | GmGmUmUmUmGfUmAfGmAm | |
| | UmUmGmApsmAps(InvAb) | | CmUpsmApsmU | |
| M25_V | (InvAb)psmApsmUmAmGmUmC | 427 | (VPmU)psfUps(UNA- | 428 |
| ar35 | mUmAfCfAfAmAmCmCmAmGm | | C)mAmAmCmUmGmGmUmUmU | |
| | UmUmGmApsmAps(InvAb) | | mGfUmAfGmAmCmUpsmApsmU | |
| M25_V | (InvAb)psmApsmUmAmGmUmC | 429 | (VPmU)psfUpsmC(UNA- | 430 |
| ar36 | mUmAfCfAfAmAmCmCmAmGm | | A)mAmCmUmGmGmUmUmUm | |
| | UmUmGmApsmAps(InvAb) | | GfUmAfGmAmCmUpsmApsmU | |
| M25_V | (InvAb)psmApsmUmAmGmUmC | 431 | (VPmU)psfUpsmCmA(UNA- | 432 |
| ar37 | mUmAfCfAfAmAmCmCmAmGm | | A)mCmUmGmGmUmUmUmGfU | |
| | UmUmGmApsmAps(InvAb) | | mAfGmAmCmUpsmApsmU | |
| M25_V | (InvAb)psmApsmUmAmGmUmC | 433 | (VPmU)psfUpsmCmAmA(UNA- | 434 |
| ar38 | mUmAfCfAfAmAmCmCmAmGm | | C)mUmGmGmUmUmUmGfUmAf | |
| | UmUmGmApsmAps(InvAb) | | GmAmCmUpsmApsmU | |
| M25_V | (InvAb)psmApsmUmAmGmUmC | 435 | (VPmU)psfUpsmCmAmAfCmUfGf | 436 |
| ar39 | mUmAfCfAfAmAmCmCmAmGm | | GmUmUmUdGfUmAfGmAmCmU | |
| | UmUmGmApsmAps(InvAb) | | psmApsmU | |
| M25_V | (InvAb)psmApsmUmAmGmUmC | 437 | (VPmU)psfUpsmCmAmAfCmUfGf | 438 |
| ar40 | mUmAfCfAfAmAmCmCmAmGm | | GmUmUmUmGdTmAfGmAmCm | |
| | UmUmGmApsmAps(InvAb) | | UpsmApsmU | |
| M25_V | (InvAb)psmApsmUmAmGmUmC | 439 | (VPmU)psfUpsmCmAmAfCmUfGf | 440 |
| ar41 | mUmAfCfAfAmAmCmCmAmGm | | GmUmUmUmGfUdAfGmAmCmU | |
| | UmUmGmApsmAps(InvAb) | | psmApsmU | |
| M25_V | (InvAb)psmApsmUmAmGmUmC | 441 | (VPmU)psfUpsmCmAmAfCmUfGf | 442 |
| ar42 | mUmAfCfAfAmAmCmCmAmGm | | GmUmUmUmGfUmAdGmAmCm | |
| | UmUmGmApsmAps(InvAb) | | UpsmApsmU | |
| M25_V | (InvAb)psmApsmGmUmCmUmAf | 443 | (VPmU)psfUpsmCmAmAfC(UNA- | 444 |
| ar43 | CfAfAmAmCfCmAmGmUmUfGps | | U)mGmGmUmUmUmGfUmAfG | |
| | mApsmA | | mAmCmUpsmApsmU | |
| M25_V | (InvAb)psmApsmGfUmCmUmAfC | 445 | (VPmU)psfUpsmCmAmAfC(UNA- | 446 |
| ar44 | fAfAmAmCfCmAmGmUmUmGps | | U)mGmGmUmUmUmGfUmAfG | |
| | mApsmA | | mAmCmUpsmApsmU | |
| M25_V | (InvAb)psmApsmGmUmCmUmAf | 447 | (VPmU)psfUpsmCmAmAfC(UNA- | 448 |
| ar45 | CfAfAmAmCfCmAmGmUmUmGp | | U)mGmGmUmUmUmGfUmAfG | |
| | smApsmA | | mAmCmUpsmApsmU | |
| M28 | mApsmApsfGmUfGmUfGmGfCm | 449 | (VPmU)psfGpsmCfCmUfAmAfUm | 450 |
| | UfCmAfUmUfAmGfGmCfApsmU | | GfAmGfCmCfAmCfAmCfUmUps | |
| | psmU | | mUpsmU | |
| M28_V | mApsmApsfGmUmGmUfGfGfCm | 451 | (VPmU)psfGpsmCmCmUfAmAm | 452 |
| ar1 | UmCfAmUmUmAmGfGmCmA | | UmGmAmGmCmCfAmCfAmCmU | |
| | | | mUpsmGpsmG | |
| M28_V | mApsmApsfGmUmGmUfGfGfCm | 453 | (VPmU)psfGpsmCmCmUfA(GNA- | 454 |
| ar2 | UmCfAmUmUmAmGfGmCmA | | A)mUmGmAmGmCmCfAmCfAm | |
| | | | CmUmUpsmGpsmG | |
| M28_V | (InvAb)psmApsmAmGmUmGmU | 455 | (VPmU)psfGpsmCpsfCmUfAmAf | 456 |
| ar3 | fGfGfCmUmCmAmUmUmAmGm | | UmGfAmGfCmCfAmCfAmCfUmU | |
| | GmCpsmAps(InvAb) | | mGpsmG | |
| M28_V | mApsmApsmGmUfGmUfGfGfCm | 457 | (VPmU)psfGpsmCmCmUfAmAfUf | 458 |
| ar4 | UmCmAmUmUmAmGmGpsmCp | | GmAmGmCmCfAmCfAmCmUmU | |
| | smA | | psmGpsmG | |
| M28_V | mCpsmCpsmAmAmGmUfGmUfG | 459 | (VPmU)psfGpsmCmCmUfAmAfUf | 460 |
| ar5 | fGfCmUmCmAmUmUmAmGmG | | GmAmGmCmCfAmCfAmCmUmU | |
| | psmCpsmA | | mGmGpsmApsmG | |
| M28_V | mCpsmApsmAmGmUmGmUmGf | 461 | (VPmU)psfGpsmCmCmUmAfAm | 462 |
| ar6 | GmCfUfCfAfUmUmAmGmGmCm | | UmGmAmGfCmCfAmCmAmCmU | |
| | Aps(InvAb) | | mUmGmGpsmUpsmU | |
| M28_V | (InvAb)psmApsmAmGmUfGmUf | 463 | (VPmU)psfGpsmCmCmUfAmAm | 464 |
| ar7 | GfGfCmUmCmAmUmUmAmGm | | UmGmAmGmCmCfAmCfAmCmU | |
| | GmCmApsmApsmC | | mUpsmGpsmG | |
| M28_V | (InvAb)psmCpsmCmAmAmGmU | 465 | (VPmU)psfGpsmCpsfCmUfAmAf | 466 |
| ar8 | mGmUfGfGfCmUmCmAmUmUm | | UmGfAmGfCmCfAmCfAmCfUmU | |
| | AmGmGmCpsmAps(InvAb) | | mGpsmG | |
| M28_V | (InvAb)psmApsmAmGmUmGmU | 467 | (VPmU)psfGpsmCmCmUfAmAfUf | 468 |
| ar9 | fGfGfCmUmCmAmUmUmAmGm | | GmAmGmCmCfAmCfAmCmUmU | |
| | GmCpsmAps(InvAb) | | mGmGpsmApsmG | |
| M28_V | mCpsmCpsmAmAmGmUfGmUfG | 469 | (VPmU)psfGpsmCpsfCmUfAmAf | 470 |
| ar10 | fGfCmUmCmAmUmUmAmGmG | | UmGfAmGfCmCfAmCfAmCfUmU | |
| | psmCpsmA | | mGpsmG | |
| M28_V | (InvAb)psmApsmAmGmUmGmU | 471 | (VPmU)psfGpsmCmCmUfAmAfUf | 472 |
| ar11- | fGfGfCmUmCmAmUmUmAmGm | | GmAmGmCmCfAmCfAmCmUmU | |
| Chol | GmCpsmAps(InvAb) | | psmGpsmG | |
| M28_V | mApsmApsmGmUfGmUfGfGfCm | 473 | (VPmU)psfGpsmCpsfCmUfAmAf | 474 |
| ar12 | UmCmAmUmUmAmGmGpsmCp | | UmGfAmGfCmCfAmCfAmCfUmU | |
| | smA | | mGpsmG | |
| M28_V | (InvAb)psmApsmAmGmUmGmU | 475 | (VPmU)psfGpsmCmCmUmAfAm | 476 |
| ar13 | fGfGfCmUmCmAmUmUmAmGm | | UmGmAmGfCmCfAmCmAmCmU | |
| | GmCpsmAps(InvAb) | | mUmGmGpsmUpsmU | |
| M28_V | mCpsmApsmAmGmUmGmUmGf | 477 | (VPmU)psfGpsmCpsfCmUfAmAf | 478 |
| ar14 | GmCfUfCfAfUmUmAmGmGmCm | | UmGfAmGfCmCfAmCfAmCfUmU | |
| | Aps(InvAb) | | mGpsmG | |
| M28_V | (InvAb)psmCpsmCmAmAmGmU | 479 | (VPmU)psfGpsmCmCmUfAmAfUf | 480 |
| ar15 | mGmUfGfGfCmUmCmAmUmUm | | GmAmGmCmCfAmCfAmCmUmU | |
| | AmGmGmCpsmAps(InvAb) | | mGmGpsmApsmG | |
| M28_V | (InvAb)psmCpsmCmAmAmGmU | 481 | (VPmU)psfGpsmCmCmUfAmAfUf | 482 |
| ar16- | mGmUfGfGfCmUmCmAmUmUm | | GmAmGmCmCfAmCfAmCmUmU | |
| | AmGmGmCpsmAps(InvAb) | | psmGpsmG | |
| M28_V | (InvAb)psmCpsmCmAmAmGmU | 483 | (VPmU)psfGpsmCmCmUmAfAm | 484 |
| ar17 | mGmUfGfGfCmUmCmAmUmUm | | UmGmAmGfCmCfAmCmAmCmU | |
| | AmGmGmCpsmAps(InvAb) | | mUmGmGpsmUpsmU | |
| M28_V | mCpsmCpsmAmAmGmUfGmUfG | 485 | (VPmU)psfGpsmCmCmUfAmAm | 486 |
| ar18 | fGfCmUmCmAmUmUmAmGmG | | UfGmAmGmCmCfAmCfAmCmU | |
| | psmCpsmA | | mUmGmGpsmApsmG | |
| M28_V | mCpsmCpsmAmAmGmUfGmUfG | 487 | (VPmU)psfGpsmCmCmUfAmAfU | 488 |
| ar19 | fGfCmUmCmAmUmUmAmGmG | | mGmAmGmCmCfAmCfAmCmU | |
| | psmCpsmA | | mUmGmGpsmApsmG | |
| M28_V | mCpsmCpsmAmAmGmUfGmUfG | 489 | (VPmU)psfGpsmCmCmUfAmAm | 490 |
| ar20 | fGfCmUmCmAmUmUmAmGmG | | UmGmAmGmCmCfAmCfAmCmU | |
| | psmCpsmA | | mUmGmGpsmApsmG | |
| M28_V | mApsmApsmGmUfGmUfGfGfCm | 501 | (VPmU)psfGpsmCmCmUfAmAm | 502 |
| ar26 | UmCmAmUmUmAmGmGpsmCp | | UmGmAmGmCmCfAmCfAmCmU | |
| | smA | | mUpsmGpsmG | |

Modification: mN = 2′OMe; fN = 2′F; ps = phosphorothioate; VP = vinyl phosphonate; invAb = inverse abasic

### 6.2 Chemical Modifications to Nucleotides

In specific embodiments, a siRNA agent described herein comprises one or more nucleotide modifications. Nucleotide modifications include, for example, end modifications, e.g., 5′-end modifications (e.g., phosphorylation, conjugation, inverted linkages) or 3′-end modifications (e.g., conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; or backbone modifications, including modification or replacement of the phosphodiester linkages. In some embodiments, a siRNA agent comprises at least one modification selected from the group consisting of modified internucleoside linkage, modified nucleobase, modified sugar, and any combinations thereof. Without limitations, such a modification can be present anywhere in the siRNA agent (e.g., in the sense strand, antisense strand, or both strands).

In some embodiments, a siRNA agent comprises one or more modified sugar modifications, such as one or more substituted sugar moieties. In some embodiments, a siRNA agent described herein includes one of the following at the 2′-position: H; F; or OCH3 (OMe).

In some embodiments, a siRNA agent described herein includes one or more glycol nucleic acids (GNA). Typically, GNA is an acyclic nucleic acid analogue, wherein its repeating glycol units are linked by phosphodiester bonds, differing from RNA's ribose sugar-phosphodiester backbone composition.

In certain embodiments, a siRNA agent described herein includes one or more terminal modifications, for example, 5′-terminal phosphorylation, conjugation, or inverted linkages. In some embodiments, the terminal modifications include a 5′-phosphate, for example, a 5′-terminal phosphate on the antisense strand of a siRNA agent.

In some embodiments, a siRNA agent includes a sense strand and/or an antisense strand with an inverted abasic nucleotide. In one embodiment, the sense strand contains an inverted abasic nucleotide at 3′ end. In another embodiment, the sense strand contains an inverted abasic nucleotide at 5′ end. In some embodiments, the sense strand contains an inverted abasic nucleotide at the 5′ and 3′ end.

In some embodiments, a siRNA agent comprises a phosphate or phosphate mimic at the 5′-end of the antisense strand. In one embodiment, the phosphate mimic is a 5′-vinyl phosphonate (VP).

In some embodiments, a siRNA agent comprises one or more modified nucleotides. In specific embodiments, the modified nucleotides are selected from the group consisting of a 2′O-methyl modified nucleotide, a deoxy-nucleotide, a 2′-fluoro modified nucleotide, a 2′-O-methyl-uridine, a 3′-O-methyl modified nucleotide, a 3′-O-methyl modified nucleotide with 2′-5′ linked phosphate, an inverted abasic nucleotide, a nucleotide comprising S-glycol nucleic acid (GNA), an unlocked nucleotide, a 5′-vinylphosphonate-2′-O-methyl-uridine, a cis-cyclobutyl phosphonate modified nucleotide, a 5′-cis-cyclobutyl phosphonate-2′-O-methyl modified nucleotide, a (L)-α-threofuranosyl modified nucleotide, and combinations thereof.

In some embodiments, a siRNA agent comprises one or more modified internucleoside linkages (i.e., a modified RNA backbone). Modified internucleoside linkages include, for example, phosphorothioates (e.g., phosphoromonothioates). Various salts, mixed salts and free acid forms are also included. In some embodiments, a siRNA agent described herein is in a free acid form. In other embodiments, a siRNA agent described herein is in a salt form. In one embodiment, a siRNA agent described herein is in a sodium salt form. In certain embodiments, when a siRNA agent described herein is in the salt form, cations of the salt (e.g., sodium cations) are present at the agent as counterions for substantially all of the electronegative groups (e.g., phosphodiester and/or phosphorothioate groups) present in the agent. In some embodiments, the counterion is a condensed counterion. In specific embodiments, the counterion is a condensed sodium cation. In some embodiments, the condensed counterion is hydrated. In specific embodiments, the condensed counterion is a hydrated sodium cation. Agents in which substantially all of the phosphodiester and/or phosphorothioate linkages have a counterion include not more than 5, 4, 3, 2, or 1 phosphodiester and/or phosphorothioate linkages without a counterion. In other words, the electronegative potential of the siRNA agent is neutralized or substantially neutralized by counterion condensation around the siRNA. In some embodiments, when a siRNA agent described herein is in the sodium salt form, sodium ions are present around the agent as counterions for substantially all of the phosphodiester and/or phosphorothioate groups present in the agent.

The phosphate group of an internucleoside phosphodiester linkage can be modified by replacing one of the oxygens with a different substituent. One result of this modification can be increased resistance of the oligonucleotide to nucleolytic breakdown. Another result of this modification can be increased stability of hybridized single-stranded RNA (ssRNA) in the siRNA agent. An example of a modified phosphate group includes phosphorothioates (e.g., phosphoromonothioates).

In some embodiments, a siRNA agent comprises an RNA mimetic, in which both the sugar and the internucleoside linkage, i.e., the backbone, of the nucleotide units are replaced with alternate groups. In specific embodiments, the base units are maintained for hybridization with an appropriate target sequence.

A siRNA agent described herein can contain one or more asymmetric centers and thus give rise to enantiomers, diastereomers, and other stereoisomeric configurations that may be defined, in terms of absolute stereochemistry, as (R) or (S), such as for sugar anomers, or as (D) or (L) such as for amino acids. Included in a siRNA agent provided herein are all such possible isomers, as well as their racemic and optically pure forms.

TABLE 33
The modified sense and antisense strand sequences of
lipid conjugated MAPT siRNA

| | | SEQ ID | | SEQ ID |
|---|---|---|---|---|
| Name | Sense strand | NO | Antisense strand | NO |
| M757 | mUpsmGpsmCmAmA(J2- | 503 | (VPmU)psfUpsmGmGdTmU | 504 |
| | CONC16A)fUmAfGfUfCmUmAmC | | (GNA-T)mGmUmAmGmAmCf | |
| | mAmAmAmCmCpsmApsmA | | UmAfUmUmUmGmCmApsmCp | |
| | | | smA | |
| M758 | (InvAb)psmUpsmGmCmAmA(J2- | 505 | (VPmU)psfUpsmGmGdTmU | 506 |
| | CONC16A)fUmAfGfUfCmUmAmC | | (UNA-U)mGmUmAmGmAmCf | |
| | mAmAmAmCmCpsmApsmA | | UmAfUmUmUmGmCmApsmCp | |
| | | | smA | |
| M759 | (InvAb)psmUpsmGmCmAmA(J2- | 507 | (VPmU)psfUpsmGmGdTmU | 508 |
| | CONC16A)fUmAfGfUfCmUmAmC | | (GNA-T)mGmUmAmGmAmCf | |
| | mAmAmAmCmCpsmApsmA | | UmAfUmUmUmGmCmApsmCp | |
| | | | smA | |
| M760 | (InvAb)psmUpsmGmCmAmA(J2- | 509 | (VPmU)psfUpsmGmGdTmU | 510 |
| | CONC16A)fUmAfGfUfCmUmAmC | | dTmGmUmAmGmAmCfUmAfU | |
| | mAmAmAmCmCpsmApsmA | | mUmUmGmCmApsmCpsmA | |
| M761 | (InvAb)psmUpsmGmCmAmA(J2- | 511 | (VPmU)psfUpsmGmGdTmU | 512 |
| | CONC16A)fUmAfGfUfCmUmAmC | | (TNA-U)mGmUmAmGmAmCf | |
| | mAmAmAmCmCpsmApsmA | | UmAfUmUmUmGmCmApsmCp | |
| | | | smA | |
| M762 | (InvAb)psmUpsmGmCmAmA(J2- | 513 | (VPmU)psfUpsmGmGdTmU | 514 |
| | CONC16A)fUmAfGfUfCmUmAmC | | (3mU2-5)mGmUmAmGmAmC | |
| | mAmAmAmCmCpsmApsmA | | fUmAfUmUmUmGmCmApsmC | |
| | | | psmA | |
| M763 | (InvAb)psmUpsmGmCmAmA(J2- | 515 | (ccBPmU)psfUpsmGmGmU | 516 |
| | CONC16A)fUmAfGfUfCmUmAmC | | fUmUfGfUmAmGmAmCfUmA | |
| | mAmAmAmCmCpsmApsmA | | fUmUmUmGmCmApsmCpsmA | |
| M764 | (InvAb)psmUpsmGmCmAmAmA | 517 | (VPmU)psfUpsmGmGdTmU | 518 |
| | (J2-CONC16U)mAfGfUfCmUmAm | | (UNA-U)mGmUmAmGmAmCf | |
| | CmAmAmAmCmCmApsmAps(Inv | | UmAfUmUmUmGpsmCpsmA | |
| | Ab) | | | |
| M765 | (InvAb)psmUpsmGmCmAmAmA | 519 | (VPmU)psfUpsmGmGdTmU | 520 |
| | (J2-CONC16U)mAfGfUfCmUmA | | (GNA-T)mGmUmAmGmAmCf | |
| | mCmAmAmAmCmCmApsmAps | | UmAfUmUmUmGpsmCpsmA | |
| | (InvAb) | | | |
| M766 | (InvAb)psmUpsmGmCmAmAmA | 521 | (VPmU)psfUpsmGmGdTmU | 522 |
| | (J2-CONC16U)mAfGfUfCmUmA | | dTmGmUmAmGmAmCfUmAfU | |
| | mCmAmAmAmCmCmApsmAps | | mUmUmGpsmCpsmA | |
| | (InvAb) | | | |
| M767 | (InvAb)psmUpsmGmCmAmAmA | 523 | (VPmU)psfUpsmGdGmUmU | 524 |
| | (J2-CONC16U)mAfGfUfCmUmA | | (UNA-U)mGmUmAmGmAmCf | |
| | mCmAmAmAmCmCmApsmAps | | UmAfUmUmUmGpsmCpsmA | |
| | (InvAb) | | | |
| M768 | (InvAb)psmUpsmGmCmAmAmA | 525 | (VPmU)psfUpsmGmGmUdT | 526 |
| | (J2-CONC16U)mAfGfUfCmUmA | | (UNA-U)mGmUmAmGmAmCf | |
| | mCmAmAmAmCmCmApsmAps | | UmAfUmUmUmGpsmCpsmA | |
| | (InvAb) | | | |
| M769 | (InvAb)psmUpsmGmCmAmAmA | 527 | (VPmU)psfUpsmGdGmUmU | 528 |
| | (J2-CONC16U)mAfGfUfCmUmA | | (GNA-T)mGmUmAmGmAmCf | |
| | mCmAmAmAmCmCmApsmAps | | UmAfUmUmUmGpsmCpsmA | |
| | (InvAb) | | | |
| M770 | (InvAb)psmUpsmGmCmAmAmA | 529 | (VPmU)psfUpsmGmGmUdT | 530 |
| | (J2-CONC16U)mAfGfUfCmUmA | | (GNA-T)mGmUmAmGmAmCf | |
| | mCmAmAmAmCmCmApsmAps | | UmAfUmUmUmGpsmCpsmA | |
| | (InvAb) | | | |
| M771 | (InvAb)psmUpsmGmCmAmAmA | 531 | (VPmU)psfUpsmGdGmUmU | 532 |
| | (J2-CONC16U)mAfGfUfCmUmA | | dTmGmUmAmGmAmCfUmAfU | |
| | mCmAmAmAmCmCmApsmAps | | mUmUmGpsmCpsmA | |
| | (InvAb) | | | |
| M772 | (InvAb)psmUpsmGmCmAmAmA | 533 | (VPmU)psfUpsmGmGmUdT | 534 |
| | (J2-CONC16U)mAfGfUfCmUmA | | dTmGmUmAmGmAmCfUmAfU | |
| | mCmAmAmAmCmCmApsmAps | | mUmUmGpsmCpsmA | |
| | (InvAb) | | | |
| M773 | (InvAb)psmUpsmGmCmAmAmA | 535 | (VPmU)psfUpsmGmGdTmU | 536 |
| | (J2-CONC16U)mAfGfUfCmUmA | | (UNA-U)mGmUmAmGmAmCp | |
| | mCmAmAmAmCmCmApsmAps | | sfUmAfUmUmUmGpsmCpsm | |
| | (InvAb) | | A | |
| M774 | (InvAb)psmUpsmGmCmAmAmA | 537 | (VPmU)psfUpsmGmGdTmU | 538 |
| | (J2-CONC16U)mAfGfUfCmUmA | | (GNA-T)mGmUmAmGmAmCp | |
| | mCmAmAmAmCmCmApsmAps | | sfUmAfUmUmUmGpsmCpsm | |
| | (InvAb) | | A | |
| M775 | (InvAb)psmUpsmGmCmAmAmA | 539 | (VPmU)psfUpsmGmGdTmU | 540 |
| | (J2-CONC16U)mAfGfUfCmUmA | | dTmGmUmAmGmAmCpsfUmA | |
| | mCmAmAmAmCmCmApsmAps | | fUmUmUmGpsmCpsmA | |
| | (InvAb) | | | |
| M776 | (InvAb)psmCpsmAmAmGmUmC | 541 | (ccBPmU)psfUpsmGmGmA | 542 |
| | (J2-CONC16C)mAfAfGfAmUmC | | mGdCmCmGmAmUmCmUfUmG | |
| | mGmGmCmUmCmCmApsmAps | | fGmAmCmUpsmUpsmG | |
| | (InvAb) | | | |
| M777 | (InvAb)psmCpsmAmAmGmUmC | 543 | (VPmU)psfUpsmGmGdAmG | 544 |
| | (J2-CONC16C)mAfAfGfAmUmC | | (UNA-C)mCmGmAmUmCmUf | |
| | mGmGmCmUmCmCmApsmAps | | UmGfGmAmCmUpsmUpsmG | |
| | (InvAb) | | | |
| M778 | (InvAb)psmCpsmAmAmGmUmC | 545 | (VPmU)psfUpsmGmGdAmG | 546 |
| | (J2-CONC16C)mAfAfGfAmUmC | | (GNA-C)mCmGmAmUmCmUf | |
| | mGmGmCmUmCmCmApsmAps | | UmGfGmAmCmUpsmUpsmG | |
| | (InvAb) | | | |
| M779 | (InvAb)psmCpsmAmAmGmUmC | 547 | (VPmU)psfUpsmGmGdAmG | 548 |
| | (J2-CONC16C)mAfAfGfAmUmC | | dCmCmGmAmUmCmUfUmGfG | |
| | mGmGmCmUmCmCmApsmAps | | mAmCmUpsmUpsmG | |
| | (InvAb) | | | |
| M780 | (InvAb)psmCpsmAmAmGmUmC | 549 | (VPmU)psfUpsmGdGmAmG | 550 |
| | (J2-CONC16C)mAfAfGfAmUmC | | (UNA-C)mCmGmAmUmCmUf | |
| | mGmGmCmUmCmCmApsmAps | | UmGfGmAmCmUpsmUpsmG | |
| | (InvAb) | | | |
| M781 | (InvAb)psmCpsmAmAmGmUmC | 551 | (VPmU)psfUpsmGmGmAdG | 552 |
| | (J2-CONC16C)mAfAfGfAmUmC | | (UNA-C)mCmGmAmUmCmUf | |
| | mGmGmCmUmCmCmApsmAps | | UmGfGmAmCmUpsmUpsmG | |
| | (InvAb) | | | |
| M782 | (InvAb)psmCpsmAmAmGmUmC | 553 | (VPmU)psfUpsmGdGmAmG | 554 |
| | (J2-CONC16C)mAfAfGfAmUmC | | (GNA-C)mCmGmAmUmCmUf | |
| | mGmGmCmUmCmCmApsmAps | | UmGfGmAmCmUpsmUpsmG | |
| | (InvAb) | | | |
| M783 | (InvAb)psmCpsmAmAmGmUmC | 555 | (VPmU)psfUpsmGmGmAdG | 556 |
| | (J2-CONC16C)mAfAfGfAmUmC | | (GNA-C)mCmGmAmUmCmUf | |
| | mGmGmCmUmCmCmApsmAps | | UmGfGmAmCmUpsmUpsmG | |
| | (InvAb) | | | |
| M784 | (InvAb)psmCpsmAmAmGmUmC | 557 | (VPmU)psfUpsmGdGmAmG | 558 |
| | (J2-CONC16C)mAfAfGfAmUmC | | dCmCmGmAmUmCmUfUmGfG | |
| | mGmGmCmUmCmCmApsmAps | | mAmCmUpsmUpsmG | |
| | (InvAb) | | | |
| M785 | (InvAb)psmCpsmAmAmGmUmC | 559 | (VPmU)psfUpsmGmGmAdG | 560 |
| | (J2-CONC16C)mAfAfGfAmUmC | | dCmCmGmAmUmCmUfUmGfG | |
| | mGmGmCmUmCmCmApsmAps | | mAmCmUpsmUpsmG | |
| | (InvAb) | | | |
| M786 | (InvAb)psmCpsmAmAmGmUmC | 561 | (VPmU)psfUpsmGmGdAmG | 562 |
| | (J2-CONC16C)mAfAfGfAmUmC | | (UNA-C)mCmGmAmUmCmUp | |
| | mGmGmCmUmCmCmApsmAps | | sfUmGfGmAmCmUpsmUpsm | |
| | (InvAb) | | G | |
| M787 | (InvAb)psmCpsmAmAmGmUmC | 563 | (VPmU)psfUpsmGmGdAmG | 564 |
| | (J2-CONC16C)mAfAfGfAmUmC | | (GNA-C)mCmGmAmUmCmUp | |
| | mGmGmCmUmCmCmApsmAps | | sfUmGfGmAmCmUpsmUpsm | |
| | (InvAb) | | G | |
| M788 | (InvAb)psmCpsmAmAmGmUmC | 565 | (VPmU)psfUpsmGmGdAmG | 566 |
| | (J2-CONC16C)mAfAfGfAmUmC | | dCmCmGmAmUmCmUpsfUmG | |
| | mGmGmCmUmCmCmApsmAps | | fGmAmCmUpsmUpsmG | |
| | (InvAb) | | | |
| M789 | (InvAb)psmUpsmGmCmAmA(J2- | 567 | (ccBPmU)psfUpsmGmGdT | 568 |
| | CONC16A)fUmAfGfUfCmUmAmC | | mUdTmGmUmAmGmAmCfUmA | |
| | mAmAmAmCmCpsmApsmA | | fUmUmUmGmCmApsmCpsmA | |
| M790 | (InvAb)psmCpsmAmAmGmUmC | 569 | (ccBPmU)psfUpsmGmGdA | 570 |
| | (J2-CONC16C)mAfAfGfAmUmC | | mGdCmCmGmAmUmCmUfUmG | |
| | mGmGmCmUmCmCmApsmAps | | fGmAmCmUpsmUpsmG | |
| | (InvAb) | | | |

(Modification: mN = 2′OMe; fN = 2′F; ps = phosphorothioate; VP = vinyl phosphonate; InvAb = inverse abasic; ccBP = cis-cyclobutyl phosphonate; ccBPmU is 5′-cis-cyclobutyl phosphonate-2′-O-methyl-uridine; TNA = (L)-a-threofuranosyl; 3mN2-5 = 3′-O-methyl with 2′-5′ linked phosphate; J2-CONC16U = L4; J2-CONC16A = L21; J2-CONC16C = L22; J2-CONC16G = L23)

### 6.3 Moieties Linked to Nucleotide Sequence

In some embodiments, a siRNA agent described herein is conjugated to one or more non-nucleotide groups. The non-nucleotide group can, e.g., enhance targeting, delivery or attachment of the siRNA agent. The non-nucleotide group can be covalently linked to the 3′ end, 5′ end, and/or internally to either the sense strand or the antisense strand of the siRNA agent. The non-nucleotide group can be covalently linked to the 3′ end, 5′ end, both the 3′ end and 5′ end, internally, both the 3′ end and internally, both 5′ end and internally, or at the 3′ end, the 5′ end, and internally of the sense strand and/or the antisense strand of the siRNA agent. In some embodiments, a siRNA agent described herein contains a non-nucleotide group linked to the 3′ end, 5′ end, both the 3′ end and 5′ end, internally, both the 3′ end and internally, both 5′ end and internally, or at the 3′ end, the 5′ end and internally of the sense strand. In certain embodiments, a siRNA agent described herein contains a non-nucleotide group linked to the 5′ end of the sense strand. In certain embodiments, a siRNA agent described herein contains a non-nucleotide group linked to the 3′ end of the sense strand. In certain embodiments, a siRNA agent described herein contains a non-nucleotide group linked to the sense strand internally. A non-nucleotide group may be linked directly or indirectly to the siRNA agent via a linker/linking group.

In some embodiments, a siRNA agent is linked to one or more lipid moieties, such as, e.g., a cholesterol moiety (e.g., CHOL4 shown in FIG. 3). In specific embodiments, a lipid moiety that is linked to a siRNA agent comprises one or more of the following compounds depicted FIG. 3, or a stereoisomer thereof, or a pharmaceutically acceptable salt thereof: L1, L2, L3, L4, L5, L6, L7, L8, L9, L10, L11, L12, L13, L14, L15, L16, L17, L18, L19, L21, L22, L23, and/or L24. In specific embodiments, a lipid moiety is selected from one or more of the following: L1, L2, L3, L4, L5, L6, L7, L8, L9, L10, L11, L12, L13, L14, L15, L16, L17, L18, L19, L21, L22, L23, and/or L24 (see FIG. 3). In specific embodiments, a lipophilic moiety comprises cholesterol-triethylene glycol; 1-((2R,3R,4R,5R)-4-hydroxy-5-(hydroxymethyl)-3-(((Z)-octadec-9-en-1-yl)oxy)tetrahydrofuran-2-yl)pyrimidine-2,4(1H,3H)-dione; N,N′-((((2R,3R,4R,5R)-4-hydroxy-5-(hydroxymethyl)tetrahydrofuran-2,3-diyl)bis(oxy))bis(propane-3,1-diyl))dipalmitamide; N-((2R,3R,4S,5R)-2-(2,4-dioxo-3,4-dihydropyrimidin-1(2H)-yl)-4-hydroxy-5-(hydroxymethyl)tetrahydrofuran-3-yl)palmitamide; (2R,3R,4R,5R)-2-(2,4-dioxo-3,4-dihydropyrimidin-1(2H)-yl)-4-hydroxy-5-(hydroxymethyl)tetrahydrofuran-3-yl hexadecylcarbamate; 3-(((2R,3R,4R,5R)-2-(2,4-dioxo-3,4-dihydropyrimidin-1(2H)-yl)-4-hydroxy-5-(hydroxymethyl)tetrahydrofuran-3-yl)oxy)-N-hexadecylpropanamide; 1-((2R,3R,4R,5R)-3-(2-(hexadecyloxy)ethoxy)-4-hydroxy-5-(hydroxymethyl)tetrahydrofuran-2-yl)pyrimidine-2,4(1H,3H)-dione; Hexadecyl ((2R,3R,4S,5R)-2-(2,4-dioxo-3,4-dihydropyrimidin-1(2H)-yl)-4-hydroxy-5-(hydroxymethyl)tetrahydrofuran-3-yl)carbamate; (2R,3R,4R,5R)-5-(heptadecyloxy)-2-(hydroxymethyl)-4-(2-methoxyethoxy)tetrahydrofuran-3-ol; (2R,3R,4R,5R)-4-(hexadecyloxy)-2-(hydroxymethyl)-5-methoxytetrahydrofuran-3-ol; (2R,3R,4R,5R)-5-(heptadecyloxy)-4-(hexadecyloxy)-2-(hydroxymethyl)tetrahydrofuran-3-ol; (2R,3R,4R,5R)-4,5-bis(hexadecyloxy)-2-(hydroxymethyl)tetrahydrofuran-3-ol; (2R,3R,4S,5R)-5,6-bis(hexadecyloxy)-2-(hydroxymethyl)-4-methoxytetrahydro-2H-pyran-3-ol; 1-((2R,3R,4R,5R)-3-((15-((3r,5r,7r)-adamantan-1-yl)pentadecyl)oxy)-4-hydroxy-5-(hydroxymethyl)tetrahydrofuran-2-yl)pyrimidine-2,4(1H,3H)-dione; (1s,4s)-4-heptadecanamidocyclohexane-1-carboxyl-; 16-oxo-16-(((1S,2R,4S)-1,7,7-trimethylbicyclo[2.2.1]heptan-2-yl)oxy)hexadecanoyl-; 16-(((1S,2R,4S)-1,7,7-trimethylbicyclo[2.2.1]heptan-2-yl)oxy)hexadecanoyl-; 15-((3r,5r,7r)-adamantan-1-yl)pentadecanoyl-; 2-(2-(N-hexadecylpalmitamido)-N-methylacetamido)ethanoyl-; 16-(((1R,2S,5R)-2-isopropyl-5-methylcyclohexyl)oxy)-16-oxohexadecanonyl-; 16-(((1R,2S,5R)-2-isopropyl-5-methylcyclohexyl)oxy)hexadecananonyl-; (2R,3R,4R,5R)-2-(6-amino-9H-purin-9-yl)-4-hydroxy-5-(hydroxymethyl)tetrahydrofuran-3-yl hexadecylcarbamate; (2R,3R,4R,5R)-2-(4-amino-2-oxopyrimidin-1(2H)-yl)-4-hydroxy-5-(hydroxymethyl)tetrahydrofuran-3-yl hexadecylcarbamate; (2R,3R,4R,5R)-2-(2-amino-6-oxo-1,6-dihydro-9H-purin-9-yl)-4-hydroxy-5-(hydroxymethyl)tetrahydrofuran-3-yl hexadecylcarbamate; or (2R,3R,4S,5R,6R)-4,6-bis(hexadecyloxy)-2-(hydroxymethyl)-5-methoxytetrahydro-2H-pyran-3-ol. In a specific embodiment, a lipid moiety, such as described in Section 7, is conjugated to the sense strand of a siRNA agent in the manner described in Section 7. In some embodiments, a lipid moiety that is linked to a siRNA agent comprises one or more of the following compounds depicted FIG. 3: L1, L2, L3, L4, L5, L6, L7, L8, L9, L10, L11, L12, L13, L14, L15, L16, L17, L18, L19, L21, L22, L23, and/or L24 in FIG. 3. In some embodiments, the lipid moiety is linked to the 3′ end, 5′ end, and/or internally to the sense strand.

In one embodiment, the sense strand of a siRNA agent is conjugated, directly or indirectly, to a lipid moiety at the 5′ end. In one embodiment, the first nucleotide at the 5′ end of the sense strand is connected to a lipid moiety. In one embodiment, the sense strand of a siRNA agent is conjugated, directly or indirectly, to a lipid moiety internally.

In one embodiment, the sense strand is conjugated, directly or indirectly, to a lipid moiety at the 3′ end. In one embodiment, the first nucleotide at the 3′ end of the sense strand is connected to a lipid moiety. In one embodiment, the first nucleotide at the 3′ end of the sense strand is connected to a lipid moiety via a linker. In one embodiment, the linker is a nucleotide linker of the length of 1, 2, 3, 4, or 5 nucleotides. In one embodiment, the nucleotide linker is dTdT. In one embodiment, the linker is an InvAb.

In one embodiment, the sense strand is conjugated, directly or indirectly, to a lipid moiety at the 5′ end, and the sense strand is also conjugated, directly or indirectly, to a lipid moiety at the 3′ end, each of which is as described herein and elsewhere. The lipid moiety at the 5′ end and the 3′ end can be the same or different.

In one embodiment, the sense strand is conjugated, directly or indirectly, to a lipid moiety at the 5′ end, at the 3′ end, and/or internally. The lipid moieties at the 5′ end, the 3′ end, and/or internally can be the same or different.

In some embodiments, a linking group is conjugated to the siRNA agent. The linking group facilitates covalent linkage of the agent to a targeting ligand or delivery polymer or delivery vehicle. The linking group can be linked to the 3′ end, the 5′ end, and/or internally of the siRNA agent sense strand. In some embodiments, the linking group is linked to the siRNA agent sense strand. In some embodiments, the linking group is conjugated to the 5′ end, 3′ end, and/or internally to the sense strand of a siRNA agent. In some embodiments, a linking group is conjugated to the 5′ end the sense strand of a siRNA agent. In some embodiments, a linking group is conjugated to the 3′ end of the sense strand of a siRNA agent. In some embodiments, a linking group is conjugated internally to the sense strand of a siRNA agent.

Typically, a linker or linking group is a connection between two atoms that links one chemical group (such as a siRNA agent) or segment of interest to another chemical group (such as a targeting group or delivery polymer) or segment of interest via one or more covalent bonds. A labile linkage contains a labile bond. A linkage may optionally include a spacer that increases the distance between the two joined atoms. A spacer may further add flexibility and/or length to the linkage.

Any of the siRNA agent nucleotide sequences listed in Table 1, 2, 3, 4, 5, 9, or 33, whether modified or unmodified, may contain a 3′ end, 5′ end, and/or internal targeting ligand and/or linking group. Any of the siRNA agent duplexes listed in Table 1, 2, 3, 4, 5, 9, or 33, whether modified or unmodified, may further comprise a targeting ligand and/or linking group, and the targeting ligand or linking group may be attached to the 3′ end, 5′ end, and/or internally to either the sense strand or the antisense strand of the siRNA agent duplex.

### 6.4 Synthesis of siRNA Agents

A siRNA agent can be synthesized by standard methods known in the art, such as those described in “Current protocols in nucleic acid chemistry,” Beaucage, S. L. et al. (Edrs.), John Wiley & Sons, Inc., New York, NY, USA, which is hereby incorporated herein by reference, or described herein (e.g., in Section 7, infra). For example, a siRNA agent may be prepared using a two-step procedure. First, the individual strands of the siRNA agent are prepared separately. Then, the component strands are annealed. The individual strands of the siRNA agent can be prepared using solution-phase or solid-phase organic synthesis or both. Organic synthesis offers the advantage that the oligonucleotide strands comprising unnatural or modified nucleotides can be easily prepared. Similarly, single-stranded oligonucleotides can be prepared using solution-phase or solid-phase organic synthesis or both. Equipment for such synthesis is sold by several vendors including, for example, Applied Biosystems® (Foster City, Calif.). In some embodiments, an oligonucleotide(s) of a siRNA agent described herein is synthesized so that it contains a reactive group, such as an amine group, at the 5′-end, 3′-end and/or internally. Such a reactive group may be used to subsequently attach a ligand (e.g., targeting ligand) using methods typical in the art. In some embodiments, an oligonucleotide(s) of a siRNA agent is synthesized with a linking group. This linking group can be used to conjugate a non-nucleotide group (e.g., a lipid moiety, a ligand or delivery group) to the siRNA agent. In some embodiments, an oligonucleotide(s) of a siRNA agent is synthesized by an automated synthesizer using phosphoramidites (e.g., standard phosphoramidites and non-standard phosphoramidites, which are commercially available). In a specific embodiment, a siRNA agent is produced using a method described in Section 7, infra. In another specific embodiment, a method described in Section 7, infra, is used to produce a siRNA agent described herein, including one conjugated to a non-nucleotide group, such as described in Section 6.3, supra.

### 6.5. Delivery Vehicles

In some embodiments, a delivery vehicle may be used to deliver a siRNA agent described herein to a cell or tissue.

### 6.6 Compositions

In one aspect, provided herein are compositions comprising a siRNA agent described herein (e.g., in Section 6.1, supra, or Section 7, infra). The composition may further comprise a carrier or excipient.

### 6.7 Uses of siRNA Agents

The siRNA agents and compositions of the invention may be used to inhibit the expression of a MAPT gene, e.g., a human MAPT gene. As shown in the Examples, the siRNA agents inhibit expression of the MAPT gene. The siRNA agents and compositions of the invention may also be used to inhibit the levels of Tau. As shown in the Examples, the siRNA agents inhibit Tau levels. Certain assays and methods for measuring MAPT expression levels and for measuring tau levels are described here. Other assays for measuring MAPT expression levels and for measuring tau levels are known in the art.

Inhibition of MAPT gene expression and/or inhibition of Tau levels is useful for studying diseases, such as Alzheimer's Disease. Tau levels have been associated with Alzheimer's disease. The siRNAs of the invention may be used to study how levels of MAPT gene expression inhibition impact Tau levels. The siRNAs of the invention may be used in animal models to study how levels of MAPT gene expression inhibition impact Tau levels. The siRNAs of the invention may be used to study how levels of MAPT gene expression and/or Tau levels impact one or more symptoms of Alzheimer's Disease.

In one aspect, provided herein is a method for inhibiting the expression of a MAPT gene (e.g., human MAPT gene) in a cell, comprising contacting a cell with a siRNA agent described herein. or a composition comprising a siRNA agent described herein. In a specific embodiment, provided herein is a method for inhibiting the expression of a MAPT gene (e.g., human MAPT gene) in a population of cells, comprising contacting a population of cells with a siRNA agent described herein. The contact between cell(s) and a siRNA agent described herein may be direct or indirect. For example, the cell(s) may be put into physical contact with the siRNA agent, or the cell(s) may be put into a situation that will permit or cause it to subsequently come into contact with the siRNA agent.

The expression of a MAPT gene (e.g., human MAPT gene) may be measured directly or indirectly. For example, the levels of MAPT RNA (e.g., pre-mRNA levels, mRNA levels, or both), the levels of a protein encoded by a MAPT gene, a function(s) of the protein encoded by a MAPT gene, or a combination thereof may be measured. Alternatively, or in addition, the expression of genes whose expression is indirectly or directly impacted by the expression of a MAPT gene, a function of a protein, which is indirectly or directly impacted by the expression of the MAPT gene, or a combination thereof may be measured. See, e.g., Section 6.8, infra, for ways that may be used to measure the expression of a MAPT gene directly or indirectly.

### 6.8 BIOLOGICAL ASSAYS

Biological assays known to one of skill in the art or described herein (e.g., in Section 7, infra) may be used to assess the ability of a siRNA agent described herein to inhibit MAPT gene expression, the specificity of a siRNA agent described herein, the stability of a siRNA agent described herein, the off-target effects of a siRNA agent described herein, the toxicity of a siRNA agent described herein, the localization of a siRNA agent to specific tissues, the pharmacokinetics of a siRNA agent, and the immunogenicity of a siRNA agent described herein.

In some embodiments, MAPT gene expression is measured at the RNA or protein level. The expression level of MAPT gene can be assessed by any method known in the art for measuring RNA. In certain embodiments, the RNA may be isolated from samples by RNA extraction methods, for example, organic extraction, membrane-based spin column, and paramagnetic particle technology. In certain embodiments, MAPT RNA levels may be measured by UV spectroscopy, in situ hybridization, fluorescent in situ hybridization (FISH), northern blotting, microarray, RT-PCR, qPCR, fluorescent dye-based quantification, and gel electrophoresis. The MAPT protein expression levels can be measured by any method known in the art for characterizing proteins. In certain embodiments, MAPT protein can be purified by salt precipitation, dialysis, and chromatography (e.g., gel filtration, ion exchange, affinity purification). In certain embodiments, crude samples containing MAPT protein or purified MAPT protein can be characterized by UV absorbance and spectroscopy, electrophoresis, capillary electrophoresis, chromatography (e.g., gel filtration, high performance liquid chromatography (HPLC), thin layer chromatography (TLC), hyperdiffusion chromatography), mass spectrometry. In certain embodiments, crude samples containing MAPT protein or purified MAPT protein can be characterized by flow cytometry, immunodiffusion, immunoelectrophoresis, western blot, immunoblot, radioimmunoassay (RIA), enzyme-linked immunosorbent assays (ELISAs), immunofluorescent assays, electrochemiluminescence assays, and the like. In some embodiments, the function of the protein encoded by the MAPT gene may be assessed to measure expression of the gene. For example, microtubule polymerization and/or microtubule stabilization by the protein encoded by the MAPT gene may be assessed. Cells contacted in vitro or ex vivo with a siRNA agent may be assessed for expression of the gene.

In some embodiments, a function(s) of the Tau protein encoded by the MAPT gene (e.g., microtubule stabilization) is assessed using an assay known to one of skill in the art. Multiple functions have been proposed for normal Tau with the most established one being microtubule stabilization.

In some embodiments, the MAPT gene expression level may be assessed using indirect measurement including expression of certain genes, functional assays of downstream proteins, and reporter assays. In some embodiments, the expression of the MAPT gene may be assessed by detecting the expression of a protein regulated by the expression of the MAPT gene. In certain embodiments, the expression of the MAPT gene may be assessed by detecting the function of a protein regulated by the expression of the MAPT gene. In some embodiments, a reporter gene can be introduced to facilitate indication of target expression. For example, a reporter gene regulated by a protein downstream of the protein encoded by the MAPT gene can indicate the expression level of the MAPT gene. Reporter genes may include, FLAG-tag, green fluorescence protein (GFP), the chloramphenicol acetyltransferase gene (cat) from Tn9 of E. coli, the luciferase gene (luc) from firefly Photinus pyralis, and β-glucuronidase (GUC). The easily detectable proteins encoded by the reporter gene indicate the MAPT gene expression level. The reporter assays may include fluorescence-based assays, for example, fluorescence spectroscopy, fluorescence immune assays, flow cytometry, fluorescence spectrometry, and ELISA.

In some embodiments, the specificity of a siRNA agent may be analyzed by the effect generated by the inhibited genes it targets or by the off-target genes that are affected. The effect includes inhibition of the MAPT gene expression, wherein the siRNA agent displays its potency by a half maximal inhibitory concentration, i.e., IC₅₀. The candidate siRNA agents are selected using IC₅₀ value in terms of effectiveness. In some embodiments, a siRNA agent described herein has an IC₅₀ of 10 nM to 200 nM in neurons (e.g., human iPSC-neurons). In certain embodiments, the specificity of the siRNA agents is tested using a negative control, wherein a scrambled or random sequence may be used. The siRNA agent that does not affect the negative target may be qualified as specific. In certain embodiments, the specificity of the siRNA agents is tested using a single target gene. Different siRNA agents to the same gene with comparable gene inhibitory efficacy should induce similar changes in gene expression profiles or phenotypes. Any changes induced by one siRNA agent and not the other(s) may be attributed to off-target effects or non-specificity. In certain embodiments, the specificity of the siRNA agents is tested using titration. Non-specific effect is mitigated when siRNA agents are used at lower concentrations. The siRNA agent that displays a lower effective concentration may have a higher specificity. In certain embodiments, the specificity of the siRNA agents is tested by monitoring both RNA and protein levels. For example, RNA gene reduction seen without a corresponding reduction in protein levels indicates that protein turnover is slow. The siRNA agent being investigated may have off-target effects. In some embodiments, a transcriptome assay may be used to assess off-target effects.

In some embodiments, the Forster resonance energy transfer (FRET) method based on agarose gel electrophoresis is to evaluate the stability of a siRNA agent described in a biological sample or fluid, such as, e.g., serum or cerebrospinal fluid sample. See, e.g., Tuttolomondo and Ditzel, 2021, Methods Mol Biol 2282:43-56 for a description of such an assay. In some embodiments, such an assay is also used to evaluate the interaction of siRNA agent described herein with serum proteins and enzymes. In certain embodiments, an agarose gel shift assay is used to evaluate the stability of a siRNA agent described herein in a biological sample or fluid, such as, e.g., serum or cerebrospinal fluid sample.

In some embodiments, the stability of a siRNA agent may be investigated under a variety of conditions, including temperature, and RNase degradation in biological fluids. In some embodiments, thermal melting temperature of a siRNA agent is measured with equimolar concentrations of both strands by monitoring A₂₆₀ with increasing temperature (e.g., 1° C./min). Tₘ is measured as the maximum of the first derivative of the melting curve (A₂₆₀ vs T) of pre-hybridized duplexes. A stable unmodified RNA may have a Tₘ ranging from 40° C. to 60° C. in terms of the thermal stability. A modified RNA may be stabilized or destabilized by −10° C. In some embodiments, the stability of a siRNA agent toward RNase degradation is determined by incubating with different biological fluid. For example, the serum stability can be determined by incubating with fetal bovine serum or human serum at 37° C. for various time periods up to 48 hours. The RNA in the samples can be examined by northern blot, in situ hybridization, qPCR, and any RNA detecting methods known in the art. The activity of siRNA agent can be tested. A stable siRNA agent may show minor degradation at 37° C. for 24 hours. In some embodiments, a siRNA agent shows sufficiently stable for at least 2 hours. In other embodiments, the stability of a siRNA agent exposed to, but not limited to, skin, salvia, topical creams or nanoparticles may be tested.

In specific embodiments, stability of siRNA agents described herein in human liver lysosomes and rat liver tritosomes are assessed as described in Section 7, infra.

In some embodiments, the toxicity of a siRNA agent to in vitro or ex vivo cell-based models is investigated. In certain embodiments, the toxicity is evaluated by the degree to which the siRNA agent can cause damage to a cell. The damage can be necrosis (uncontrolled cell death), apoptosis (programmed cell death), autophagy, or stop actively growing and dividing to decrease cell proliferation. In certain embodiment, cytotoxicity assays are performed to measure the ability of the siRNA agent to cause cell damage or cell death. For example, the release of lactate dehydrogenase (LDH) and glucose 6-phosphate dehydrogenase (G6PD) can be used as biomarkers for cellular plasma membrane damage. In another embodiment, cell viability under the treatment of the siRNA agent can be detected by various mechanisms, for example, membrane integrity, enzyme activity, or metabolic activity.

In some embodiments, RNA in situ hybridization assays may be used to image a siRNA agent and to assess the inhibition of MAPT gene expression in tissues samples from a subject. RNAscope® may be used, e.g., for RNA in situ hybridization assays. In some embodiments, a siRNA agent described herein is used to study Alzheimer's disease an animal model (e.g., a mouse model). Animal models are known in the art. In a specific embodiment, the animal model is one described in Section 7, infra.

### 7. EXAMPLES

### 7.1 Example 1: siRNA Synthesis

Table 1 provides the unmodified sense and antisense strand nucleotide sequences of siRNAs targeting human MAPT, Gene ID: 4137. Table 2 provides chemical modifications, including 2′-Fluoro, 2′-O-Methyl, and phosphorothioate, applied to the siRNA sequences in Table 1.

TABLE 1
UNucleotide nmodified Sense Strand and
Antisense Strand Sequences (5′ to 3′)

| | | SEQ ID | | SEQ ID |
|---|---|---|---|---|
| Name | Sense strand | NO | Antisense strand | NO |
| UM25 | AGUCUACAAACCAGUUGAC | 1 | GUCAACUGGUUUGUAGACU | 2 |
| UM28 | AAGUGUGGCUCAUUAGGCA | 3 | UGCCUAAUGAGCCACACUU | 4 |
| UM96 | AGAUCACGCUGGGACGUAC | 5 | GUACGUCCCAGCGUGAUCU | 6 |
| UM165 | AGUCCAAGAUCGGCUCCAA | 7 | UUGGAGCCGAUCUUGGACU | 8 |
| UM177 | CAAAUAGUCUACAAACCAA | 9 | UUGGUUUGUAGACUAUUUG | 10 |

TABLE 2
Modified Sense Strand and Antisense Strand Nucleotide
Sequences (5′ to 3′)

| | | SEQ ID | | SEQ ID |
|---|---|---|---|---|
| Name | Sense strand | NO | Antisense strand | NO |
| M25 | mApsmGpsfUmCfUmAfCmAfAm | 11 | fUpsfUpsmCfAmAfCmUfGmG | 12 |
| | AfCmCfAmGfUmUfGmAfApsmU | | fUmUfUmGfUmAfGmAfCmUps | |
| | psmU | | mUpsmU | |
| M28 | mApsmApsfGmUfGmUfGmGfCm | 13 | fUpsfGpsmCfCmUfAmAfUmG | 14 |
| | UfCmAfUmUfAmGfGmCfApsmU | | fAmGfCmCfAmCfAmCfUmUps | |
| | psmU | | mUpsmU | |
| M96 | mApsmGpsfAmUfCmAfCmGfCm | 15 | fUpsfUpsmAfCmGfUmCfCmC | 16 |
| | UfGmGfGmAfCmGfUmAfApsmU | | fAmGfCmGfUmGfAmUfCmUps | |
| | psmU | | mUpsmU | |
| M165 | mApsmGpsfUmCfCmAfAmGfAm | 17 | fUpsfUpsmGfGmAfGmCfCmG | 18 |
| | UfCmGfGmCfUmCfCmAfApsmU | | fAmUfCmUfUmGfGmAfCmUps | |
| | psmU | | mUpsmU | |
| M177 | mCpsmApsfAmAfUmAfGmUfCm | 19 | fUpsfUpsmGfGmUfUmUfGmU | 20 |
| | UfAmCfAmAfAmCfCmAfApsmU | | fAmGfAmCfUmAfUmUfUmGps | |
| | psmU | | mUpsmU | |

(Modification: mN = 2′OMe; fN = 2′F; ps = phosphorothioate)

### Synthesis of MAPT siRNA Duplex

The human MAPT siRNA were synthesized and annealed as below. Briefly, single-stranded sense and antisense oligonucleotides were synthesized at 0.2 mol scale on a Dr. Oligo 192 synthesizer (Biolytic Lab Performance) using the standard solid-phase phosphoramidite chemistry. Controlled pore glass (CPG, 500 Å) was used as solid support loaded with first base or UnyLinker™. 3% Dichloroacetic acid in Dichloromethane was used for detritylation. 2′-OMe and 2′-F modified nucleotides were coupled by using corresponding phosphoramidites. Coupling time for all phosphoramidites was 6-8 minutes using 5-Ethylthio-1H-tetrazole (ETT) as activator (0.25 M in acetonitrile). Phosphorothioate linkages were generated using a 0.2 M solution of xanthane hydride in anhydrous pyridine. The oxidation time was 3 minutes using 0.02 M I₂ in tetrahydrofuran with 10% water.

After synthesis, the solid support was transferred to a 1.5 mL vial. Cleavage and deprotection were performed using 500 μL AMA (concentrated ammonia/40% aqueous methylamine, v/v=1:1). After the completion of cleavage and deprotection, the sample was filtrated to remove the solid support and washed once with water. The sample was purified using a 6 mL Source 15Q ion exchange column (Cytiva) on an AKTA Purifier System equipped with autosampler and fraction collector. Fractions were analyzed by LC-MS to confirm the quality. The appropriate fractions were pooled and desalted using CentriPure P10 Columns (emp Biotech). Samples from each sequence were analyzed by LC-MS to confirm the identity, UV (260 nm) for quantification and IP-RP chromatography to determine purity. Duplex annealing was performed by mixing equimolar of sense and antisense single strands. Final duplex sample solution was dried under vacuum using GeneVac evaporator (SP Scientific). The final QC specifications are ±0.05% of calculated mass (by LC/MS) for single strand identity, >85% full length oligonucleotide (by HPLC) for single strand purity, and >90% by non-denaturing HPLC for duplex purity.

siRNA-Lipid Conjugation

A subset of the siRNA compounds was synthesized with different novel lipid conjugates and at different positions (5′/3′-end, or internal). Lipids were incorporated to the sense strands via on-column and/or post-column synthesis. To perform on-column synthesis, lipid moiety was modified at the corresponding phosphoramidite monomer or CPG, followed by solid-phase phosphoramidite chemistry, as described above, to synthesize sense strand with 5′/3′ terminal and/or the internal lipid conjugates. For post-column synthesis the lipids were introduced in solution phase with their corresponding N-hydroxysuccinimide esters and the appended amine linker of the nucleotide.

### On-Column Synthesis

For example, on-column introduction of compound 2 at the 3′ end of the sense strand was performed using 3′-amino modifier such as 2-dimethoxytrityloxymethyl-6-fluorenylmethoxycarbonylamino-hexane-1-succinoyl)-long chain alkylamino-CPG 1 (Scheme 1). Following the deprotection of the Fmoc group with 20% piperidine in DMF the CPG was thoroughly washed with DMF, MeCN, diethyl ether, and dried. A solution of 80 μmol of compound 2 in 2 ml of 1,4-dioxane was added to the above CPG followed by 30 μl of DIPEA and shaken for 12 hours to produce compound 3. The CPG was washed with DCM, MeCN, and Et₂O. A solution of 1 ml of CAP A and CAP B was added and shaken for 30 minutes. Compound 3 was washed with DCM, MeCN, Et₂O, and dried and used for the oligonucleotide synthesis. To synthesize oligonucleotide conjugated with cholesterol at 3′ end, 3′-Cholesteryl-TEG CPG was used.

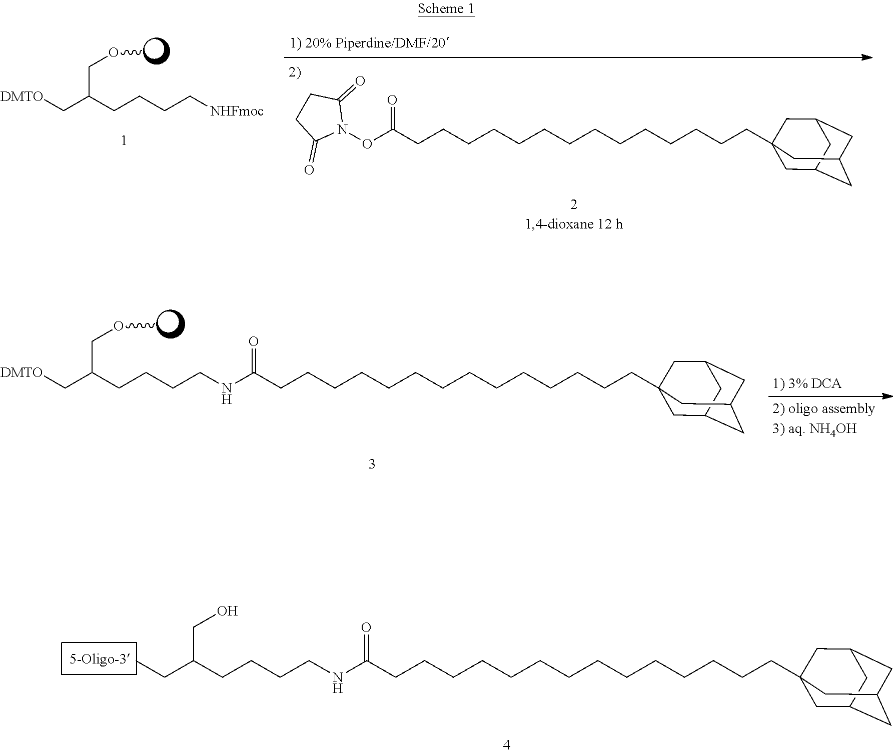

Introduction of compound 2 at the 5′ end of the sense strand was carried out using 5′-amino modifier such as 6-(4-monomethoxytritylamino)hexyl-(2-cyanoethyl)-(N,N′-diisopropyl)-phosphoramidite. After the synthesis of the sense strand on the CPG following the standard procedure, in the penultimate step the 5′ end of the sense strand was coupled to 6-(4-monomethoxytritylamino)hexyl-(2-cyanoethyl)-(N,N-diisopropyl)-phosphoramidite followed by deblocking of 4-monomethoxytrityl group with 3% DCA/DCM for 30 minutes. The CPG was subsequently washed with DCM, ACN and coupled with compound 2 for 12 hours (Scheme 2), followed by strand cleavage and deprotection.

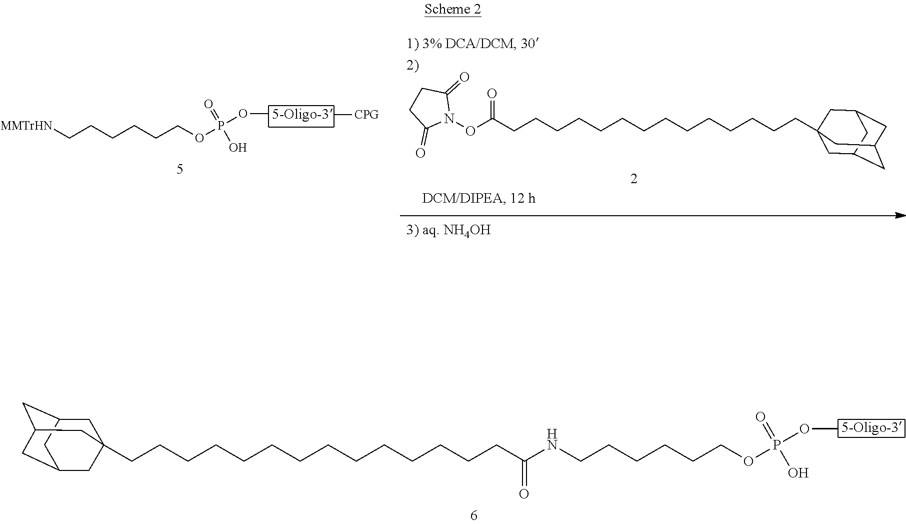

The adamantly lipid at the internal position was introduced during the sense strand synthesis with corresponding phosphoramidite monomer. For example, Scheme 3 illustrates this with uridine based phosphoramidite monomer 7.

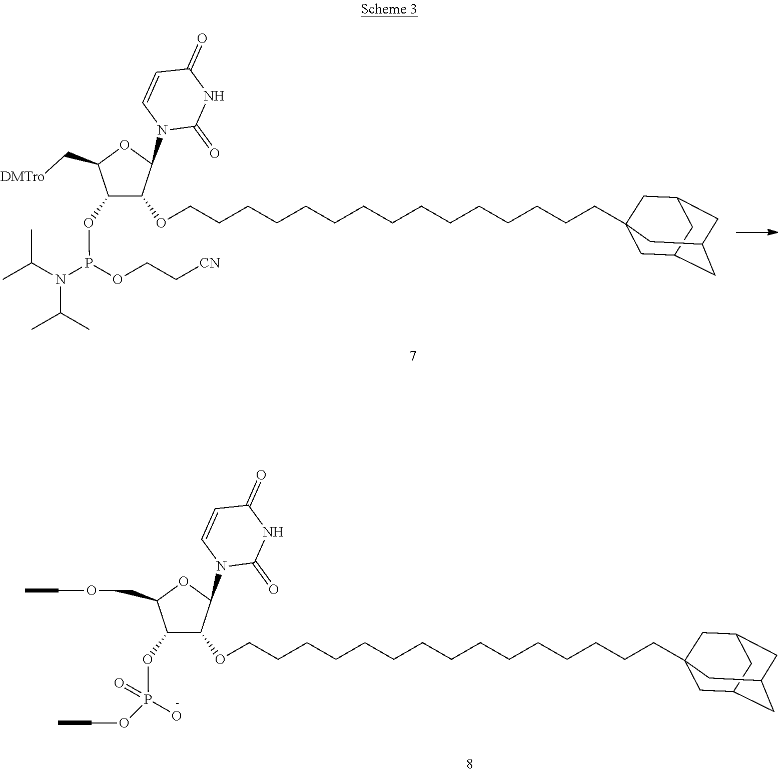

The synthesis, cleavage and deprotection, the lipid conjugated oligonucleotides were purified by reversed-phase or ion-exchange chromatography. The buffers for reverse phase were 0.05 M sodium acetate in 90/10% water/acetonitrile (Buffer A) and acetonitrile (Buffer B), and the buffers for ion-exchange are 20 mM sodium phosphate in 90/10% water/acetonitrile (buffer A) and 20 mM sodium phosphate in 90/10% water/acetonitrile, 1.8 M sodium bromide (buffer B). Fractions containing full-length oligonucleotides were pooled, desalted, and the compounds were analyzed by LC-MS.

### Post-Column Synthesis

Lipids were conjugated at the 5′/3′ terminal position and at the internal position of the sense strand in the solution phase after the purification and desalting of the corresponding oligonucleotide.

For example, incorporation at the internal position was carried out with phosphoramidite monomer derivatized with an amine linker at the 2′ position of respective nucleotide. Scheme 4 illustrates this with uridine based phosphoramidite monomer compound 9. After incorporation of compound 9 at the internal position and complete synthesis, cleavage and deprotection, purification and desalting process, the free oligonucleotide with the pendant amine was coupled with corresponding lipid-NHS esters in solution phase.

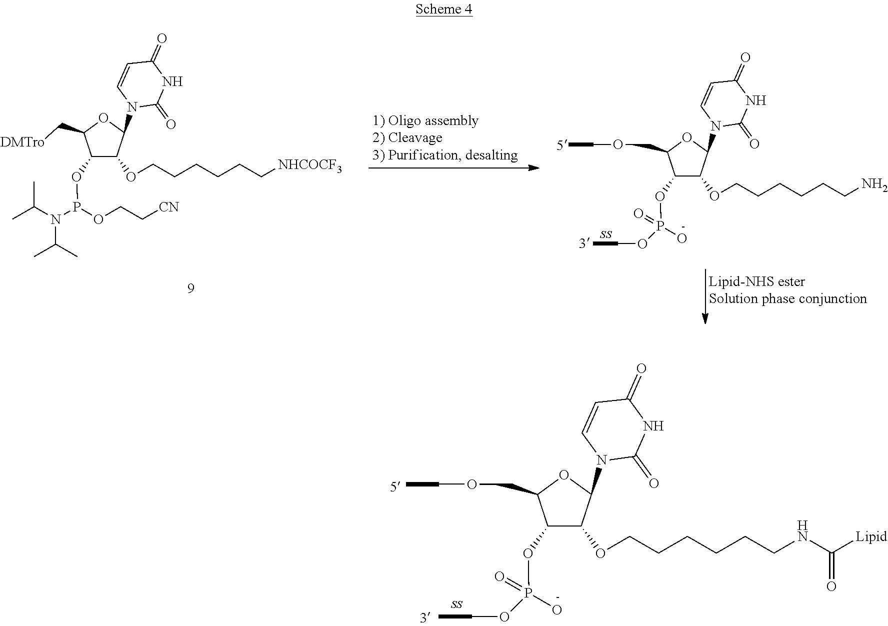

Conjugation at the terminal/internal positions was carried out with 0.15 mM solution of deprotected and desalted oligonucleotide in 0.1 M solution of NaHCO₃ (pH 8.4) with 1.5 mM solution of the corresponding lipid-NHS ester in DMF at 60° C. To a solution (0.25 mL) of oligonucleotide was added DMF solution of lipid-NHS ester (0.6 mL) and the resulting mixture heated at 63° C. Progress was monitored by RP-HPLC (C-18 column, A: 50 mM TEAA, B: MeCN; gradient 5-100% B at 30° C.). Reaction was >90% completed usually in less than one hour. Reaction mixture was diluted with water and purified by RP-HPLC (C-8 Xbridge Waters column; A: 50 mM NaOAc, B: MeCN; 5-100% B gradient, at 60° C.). Isolated yields of lipid conjugated oligonucleotides were 60-70%. After purification, the products were desalted and duplex annealing, as described above.

### 7.2 Example 2: In Vitro Screening of Mapt siRNAs

The screenings of MAPT siRNA agents listed in Table 2 were performed in human neuroblastoma cells with transfection following the protocol described below. Table 6 shows the remaining MAPT mRNA in human neuroblastoma cells after transfection of siRNAs at concentrations of 20 nM and 200 pM. A set of 25 siRNA hits were selected for potency assessment in human neuroblastoma cells with transfection at 4-fold serial dilutions at concentrations of 2 nM, 500 pM, 125 pM, 31.25 pM, 7.81 pM, 1.95 pM, 0.49 pM, 0.12 pM, 0.03 pM and 0.0076 pM. Table 7 summarizes the IC₅₀ values and their 95% confidential intervals of indicated siRNAs.

TABLE 6
Screening of MAPT siRNAs in human neuroblastoma cells
with transfection. Data are expressed as percentage
of MAPT mRNA level relative to vehicle control.

| | |
|---|---|
| 20 nM | 200 pM |

| Name | mean | SD | mean | SD |
|---|---|---|---|---|
| M25 | 12.9 | 0.007 | 30.3 | 0.5 |
| M28 | 9.4 | 0.1 | 26.0 | 1.5 |
| M96 | 19.2 | 1.7 | 34.7 | 2.0 |
| M165 | 22.6 | 0.2 | 29.5 | 3.6 |
| M177 | 11.9 | 1.8 | 22.1 | 0.2 |

### Cell Culture and Transfections:

Human neuroblastoma Kelly cells (DSMZ) were cultured in RPMI 1640 (Sigma R0883) supplemented with 10% FBS (Biowest S1810-500), 2 mM L-Glutamine (Sigma G7513) and 50 g/mL Gentamycin (Gibco 15750) at 37° C. incubator with 5% CO₂. One day before transfection, cells were split using 0.05% Trypsin-EDTA (Gibco 25300) and plated at a density of 30,000 cells per well in 96-well plates. The next day, cells were transfected with siRNA using Lipofectamine RNAiMAX Transfection Reagent (Thermo Fisher Scientific 13778150) following manufacture's protocol. Prior to RNA isolation, cells were cultured at 37° C. incubator in 5% CO₂ for 72 hours.

### Total RNA Isolation:

RNA extraction was performed using the RNeasy 96 kit (Qiagen) following manufacture's protocol. Briefly, 125 μL RLT buffer was added to each well of 96-well plate to lysate cells with orbital shaking at 200 rpm for 1 min, cell plates were then transferred to −80° C. freezer for storage till analysis. On the day of RNA extraction, frozen cell plates were quickly thaw in 37° C. oven, immediately after thawing, an equal volume 125 μL of 70% (V/V) ethanol was added to each well to mix with RLT lysate. The mixture was subsequently transferred to wells of the RNeasy 96 plate placed on top of QIAvac 96 vacuum block, and RNA was bound to the RNeasy 96 plate membrane by applying vacuum till liquid transfer was complete. Serial wash steps including 1 time of 800 μL RW1 buffer, 2 times of 800 μL RPE buffer were applied to the RNeasy 96 plate using vacuum to remove the liquid. After the last wash, the RNeasy 96 plate was centrifuged at 5600×g for 3 min to remove the residual lipid. RNA was eluted using 60 μL RNase-free water by centrifugation at 5600×g for 3 min at RT. RNA concentration was measured by Nanodrop 8000 (ThermoFisher). The eluted RNA was stored at −80° C. until analysis.

### Reverse Transcription and Real Time PCR:

RNA was used as template for reverse transcription using reversed transcribed using High-Capacity cDNA Reverse Transcription Kits (Applied Biosystems) following manufacture's protocol. Briefly, a final reaction of 20 μL mixture was incubated at 25° C. for 10 min, followed by reverse transcription at 37° C. for 2 hours and enzyme inactivation at 85° C. for 5 min. For qPCR reaction, reverse transcribed cDNAs were diluted 10 times and mixed with 2× PowerUp™ SYBR™ Green Mater Mix (ThermoFisher A25743) and 500 nM qPCR primers to a final volume of 10 μL reaction. The qPCR runs were performed with a QuantStudio™ 12K instrument (Applied Biosystems™) using standard thermal cycling protocol. Multiple validated primers were used to detect MAPT mRNA, and reference primers targeting housekeeping genes ENOX2 and GAPDH were used for normalization of gene expression. The primer sequences were listed in Table 8. qPCR data were analysed using qBase+ software (Biogazelle). IC₅₀ values and the 95% CI of IC₅₀ were calculated from a log([drug]) versus normalized response curve fit using GraphPad Prism version 7.00 Software.

### 7.3 Example 3: In Vitro Knockdown Efficiency Assessment of siRNA Conjugates in Human iPSC-Derived Neurons

The in vitro knockdown efficiencies of lipid-siRNA conjugates were assessed in human iPSC-derived cortical neurons following the procedure described below.

Tables 18-19 show that cholesterol-siRNA conjugates listed in Table 9 dose-dependently knock down MAPT mRNA and Tau protein in human iPSC-neurons after incubation for 7 days or 14 days. Data are presented as remaining MAPT mRNA (relative to control) or remaining Tau protein (relative to control). Mean±S.D., n=3. The IC₅₀ (95% CI) values are calculated from a log([drug]) versus normalized response curve fit using GraphPad Prism version 7.00 Software.

TABLE 9
The sequences and modifications of the
cholesterol-siRNA conjugates
Modified sequences (5′ to 3′)

| | Sense strand sequence | SEQ ID | Antisense strand | SEQ ID |
|---|---|---|---|---|
| NAME | (5′- 3′) | NO | sequence (5′-3′) | NO |
| M25- | mApsmGpsfUmCfUmAfCmAfAm | 167 | (VPmU)psfUpsmCfAmAfCmUfG | 168 |
| Chol | AfCmCfAmGfUmUfGmAfApsmU | | mGfUmUfUmGfUmAfGmAfCmUps | |
| | psmU(Chol4) | | mUpsmU | |
| M28- | mApsmApsfGmUfGmUfGmGfCm | 169 | (VPmU)psfGpsmCfCmUfAmAfU | 170 |
| Chol | UfCmAfUmUfAmGfGmCfApsmU | | mGfAmGfCmCfAmCfAmCfUmUps | |
| | psmU(Chol4) | | mUpsmU | |
| M28_V | mApsmGpsfUmCmUmAfCfAmAf | 171 | (VPmU)psfGpsmCmCmUfAmAmU | 172 |
| ar1- | mAmCfCmAmGmUmUfGmAmA | | mGmAmGmCmCfAmCfAmCmU | |
| Chol | (Chol4) | | mUpsmGpsmG | |
| M28_V | mApsmApsfGmUmGmUfGfGfCm | 173 | (VPmU)psfGpsmCmCmUfA(GNA- | 174 |
| ar2- | UmCfAmUmUmAmGfGmCmA | | A)mUmGmAmGmCmCfAmCfAm | |
| Chol | (Chol4) | | CmUmUpsmGpsmG | |
| MsiR2 | mGpsfCpsmAfUmGfGmUfCmAf | 175 | (VPmU)psfUpsmUfGmCfUmUfU | 176 |
| 66- | GmUfAmAfAmAfGmCfAmAfAps | | mUfAmCfUmGfAmCfCmAfUmGfC | |
| Chol | mUpsmU(Chol4) | | psmUpsmU | |
| NTC- | mApsfGpsmUfAmCfUmGfCmUf | 177 | (VPmU)psfCpsmGfUmAfUmCfG | 178 |
| Chol | UmAfCmGfAmUfAmCfGmGpsmU | | mUfAmAfGmCfAmGfUmAfCmUps | |
| | psmU(Chol4) | | mUpsmU | |

Modification:
mN = 2′OMe;
fN = 2′F;
ps = phosphorothioate;
VP = vinyl phosphonate;
invAb = inverse abasic

TABLE 18
Knockdown of MAPT mRNA in human iPSC-neurons

| | IC₅₀ (95% CI) |
|---|---|
| M25-Chol | 3.557e−008 to 4.567e−008 |
| MsiR266-Chol | 2.281e−008 to 3.319e−008 |

TABLE 19
Knockdown of MAPT mRNA in human iPSC-neurons

| | IC₅₀ (95% CI) |
|---|---|
| M28-Chol | 4.981e−009 to 6.568e−009 |

TABLE 20
Knockdown of Tau Protein in human iPSC-neurons

| | IC₅₀ (95% CI) |
|---|---|
| M25-Chol | 4.156e−008 to 6.211e−008 |
| M28-Chol | 9.552e−009 to 1.618e−008 |
| MsiR266-Chol | 1.892e−008 to 3.958e−008 |

Tables 10 and 11 summarize the efficiency of cholesterol-siRNA conjugates with various chemical modifications (listed in Table 4) in knocking down MAPT mRNA in human iPSC-neurons after incubation for 7 days. Data are presented as remaining MAPT mRNA relative to control. Mean±S.D., n=3.

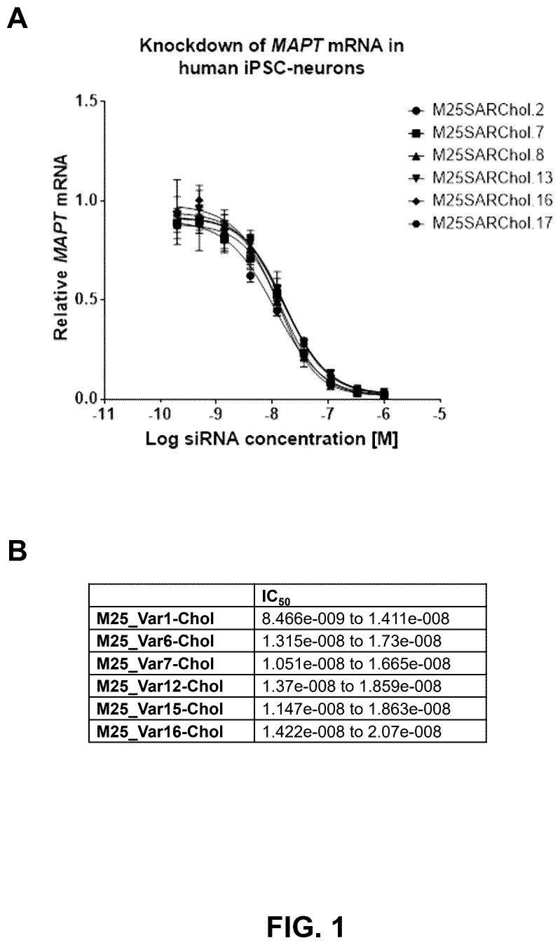

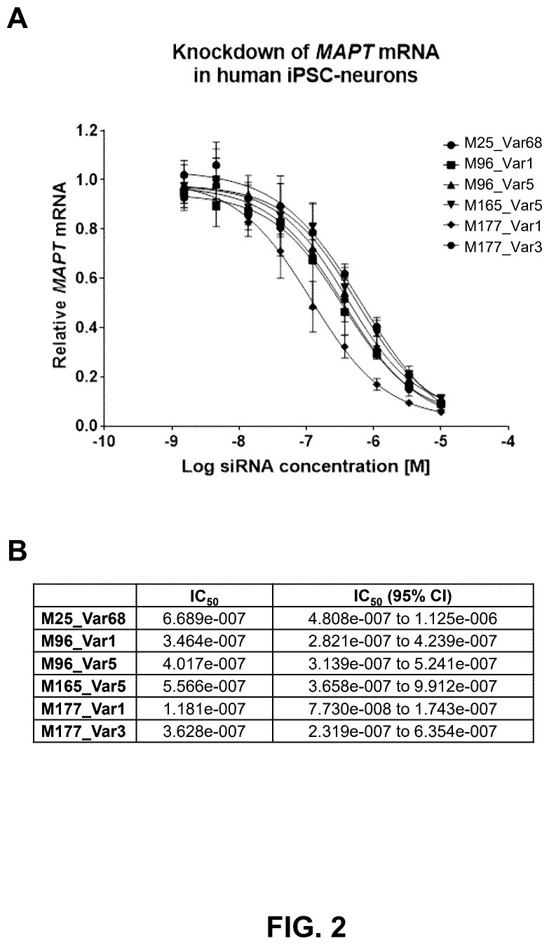

FIGS. 1A-1B and FIGS. 2A-2B show that cholesterol-siRNA conjugates or lipid-siRNA conjugates with various chemical modifications (listed in Table 4 and Table 23) dose-dependently knock down MAPT mRNA and Tau protein in human iPSC-neurons after incubation for 7 days. Data are presented as remaining MAPT mRNA (relative to control) or remaining Tau protein (relative to control). Mean±S.D., n=3. The IC₅₀ (95% CI) values are calculated from a log([drug]) versus normalized response curve fit using GraphPad Prism version 7.00 Software.

Tables 12-14 summarize the efficiency of MAPT siRNAs in conjugation with different lipids (listed in Table 5) in knocking down MAPT mRNA in human iPSC-neurons after incubation for 7 days. Three concentrations of siRNA conjugates at 1 μM, 200 nM and 40 nM were tested. Data are presented as remaining MAPT mRNA relative to control. Mean±S.D., n=3.

Differentiation of Human iPSCs into Cortical Neurons

The human iPSC line (Sigma #iPSC0028) was derived with OSKM retroviral reprogramming of epithelial cells from a 24-years old Caucasian female donor. iPSCs were first differentiated into cortical neural stem cells (NSCs) following a dual SMAD inhibitor protocol (Shi et al., 2012, 2012, Nat. Proc. 7(10):1836-46) with some modifications. Briefly, iPSCs were plated at 500,000 cells/cm² on wells coated with Matrigel (Corning 354230) in mTeSR medium (StemCell Technologies 5850) supplemented with 10 pM ROCK inhibitor (Sigma-Aldrich Y0503) and cultured at 37° C. and with 5% 02. The media were replaced with mTeSR the next day (day −1). From day 0 till day 12, cell media were changed every day with cortical Neural Induction Medium comprised of 10 M SB43142 (Tocris 1614) and 1 μM Dorsomorphin (Tocris 3093) supplemented in Neural Maintenance Medium (1:1 DMEM:F12 Glutamax (ThermoFisher Scientific 10565108), Neurobasal (ThermoFisher Scientific 21103049), 2.5 g/mL Insulin (Sigma-Aldrich 19278-5 ML), 50 uM 2-mercaptoethanol (ThermoFisher Scientific, 31350010), 0.5% Non-Essential Amino Acids (ThermoFisher Scientific 11140035), 0.5% GlutaMAX supplement (ThermoFisher Scientific, 10565018), 0.5 mM Sodium Pyruvate (ThermoFisher Scientific 11360070), 1% Penicillin-Streptomycin (Sigma P4333), 0.5% N2 supplement (ThermoFisher Scientific 17502048), 1% B27 supplement (ThermoFisher Scientific 17504044). At day 12, the neuroepithelial sheet was gently detached into large aggregates of 300 to 500 cells using a needle and lifter and the clumps were collected in a 15 mL falcon by centrifugation at 160 g for 2 min. Cell pellets were gently resuspended in Neural Induction Medium for a ½ or ⅓ passage into wells coated with 10 μg/mL laminin (Sigma-Aldrich L2020) in a total volume of 2 mL of Neural Induction Medium per well of the 6-well plate. Media were changed at day 13 and day 15 into Neural Maintenance Medium supplemented with 20 ng/mL of FGF2 (Stemcell Technologies 2634). At day 17, the neural rosettes were detached with dispase (ThermoFisher Scientific 17105041) for a ⅓ passage and plated into laminin-coated wells. Perform 1 or 2 extra dispase steps for another week. At around days 25-30, neural stem cells were dissociated with Accutase (ThermoFisher Scientific A1110501) into single-cell suspension and cryopreserved in freshly prepared Neural Freezing Medium containing Neural Maintenance Medium supplemented with 10% (V/V) DMSO and 20 ng/mL FGF2. The frozen vials of neural stem cells were stored in liquid nitrogen till use.

To generate iPSC-neurons, frozen vials of neural stem cells (NSCs) were thawed and plated on laminin-coated wells at 70,000 cells/cm² in Neural Maintenance Medium supplemented with 10 μM ROCK inhibitor and 20 ng/mL FGF2. In the following two days, media were replaced daily with neural maintenance medium. At around day 4 after thawing, cells were dissociated with Accutase and placed at 28.000 cells per well in 96-well plates pre-coated with poly-L-ornithine and laminin in Neural Maintenance Medium supplemented with 10 μM ROCK inhibitor. The next day after re-plating, culture media were replaced with Neural Differentiation Medium comprised of Neural Maintenance Medium supplemented with 20 ng/mL BDNF (R & D Systems 212-BD-050/CF), 20 ng/mL GDNF (R & D Systems 212-GD-050/CF), 500 μM DB-cAMP (Sigma D0627) and 20 mM Ascorbic Acid (Sigma A4403). Cultures were differentiated in Neural Differentiation Medium with 50% medium change twice per week. Two-to-three weeks after differentiation from neural stem cells (NSCs), neurons were treated with siRNA for 7 days or 14 days for RNA analysis by Reverse transcription and Real time PCR and protein analysis by MSD immunoassay.

### MSD Immunoassay

Human iPSC-neurons differentiated on 96-well plate were lysed in 100 μL ice-cold RIPA buffer (Sigma) supplemented with cOmplete™ Protease inhibitor cocktail (Roche) and PhosSTOP™ (Roche) with slow orbital shaking for 30 min at 4° C. The plates were centrifuged at 1,000×g for 5 min, and the cell lysates were collected and diluted for different (20, 50 or 100) folds for protein measurement using MSD immunoassays following a standardized procedure. Briefly, MSD plates were coated with 30 μL per well of coating antibody diluted in PBS at 1 μg/mL overnight at 4° C. The next day, plates were inverted on absorbent tissue to dry and afterwards incubated with 150 μL per well blocking buffer 0.1% Casein for 2 hours at RT with orbital shaking at 300 rpm. After blocking, the plates were incubated with cell lysate diluted in RIPA buffer supplemented with cOmplete™ Protease inhibitor cocktail (Roche) overnight with slow orbital shaking at 4° C. Plates were washes 5× times using Titertek Aquamax 4000 and inverted on absorbent tissue to dry. Afterwards, 25 μL/per secondary or detection antibody diluted in 0.1% casein were added to MSD plates and incubated for 2 hours at room temperature. After antibody incubation, plates were washed 5× times and developed with 150 μL/well 2×MSD reading buffer (Meso Scale Discovery) diluted in milliQ H2O. Plates were read immediately using MSD instrument. For measurement of Tau protein, hTau43 antibody (Janssen) was used for 96-well MSD plate coating and SULFO-TAG™ labeled hTau60 antibody (Janssen) was used for detection. For measurement of Histone H3 protein, recombinant rabbit monoclonal antibody 17H2L9 (ThermoFisher) was used for plate coating, mouse mAb 14221BF (Cell Signaling Technology) was used as primary detection and SULFP-TAG labeled anti-mouse antibody (Meso Scale Discovery) was used as secondary detection antibody.

### 7.4 Example 4: In Vitro Stability Assessment of siRNA Conjugates in Various Matrices

The in vitro stability of siRNA conjugates was assessed in various matrices including mouse brain homogenates, human liver lysosomes and rat liver tritosomes using the methods described below. The results of the analyses are provided in Tables 15-17.

Tables 15-17 and Tables 24-26 summarize the LC-MS measurement of antisense strand and sense strand stability of siRNA conjugates (listed in Table 4, Table 5, and Table 23) in mouse brain homogenates (Table 15 and Table 24), human liver lysosomes (Table 16 and Table 25) and rat liver tritosomes (Table 17 and Table 26) at 37° C. after 24 hours incubation with shaking at 450 rpm. A reference compound MSC-2-V was added in the assays for benchmarking the stringency of the assay conditions.

TABLE 4
Modified Nucleotide Sense and Antisense
Strand Sequences (5′ to 3′)

| | | SEQ ID | | SEQ ID |
|---|---|---|---|---|
| Name | Sense strand | NO | Antisense strand | NO |
| M25- | mApsmGpsfUmCfUmAfCmAfAm | 21 | (VPmU)psfUpsmCfAmAfC | 22 |
| Chol | AfCmCfAmGfUmUfGmAfApsmU | | mUfGmGfUmUfUmGfUmAfG | |
| | psmU(Chol4) | | mAfCmUpsmUpsmU | |
| M25_V | mApsmGpsfUmCmUmAfCfAfAm | 23 | (VPmU)psfUpsmCmAmAfC | 24 |
| ar1- | AmCfCmAmGmUmUfGmAmA | | mUmGmGmUmUmUmGfUmAfG | |
| Chol | (Chol4) | | mAmCmUpsmApsmU | |
| M25_V | (InvAb)psmApsmGmUmCmUmA | 25 | (VPmU)psfUpsmCpsfAmA | 26 |
| ar2- | fCfAfAmAmCmCmAmGmUmUmGm | | fCmUfGmGfUmUfUmGfUmA | |
| Chol | ApsmAps(InvAb)(Chol4) | | fGmAfCmUmApsmU | |
| M25_V | mApsmGpsmUmCfUmAfCfAfAm | 27 | (VPmU)psfUpsmCpsmAmA | 28 |
| ar3- | AmCmCmAmGmUmUmGpsmApsmA | | fCmUfGfGmUmUmUmGfUmA | |
| Chol | (Chol4) | | fGmAmCmUpsmApsmU | |
| M25_V | mApsmUpsmAmGmUmCfUmAfCf | 29 | (VPmU)psfUpsmCmAmAfC | 30 |
| ar4- | AfAmAmCmCmAmGmUmUmGpsmA | | mUfGfGmUmUmUmGfUmAfG | |
| Chol | psmA(Chol4) | | mAmCmUmAmUpsmUpsmU | |
| M25_V | mUpsmApsmGmUmCmUmAmCfAm | 31 | (VPmU)psfUpsmCmAmAmC | 32 |
| ar5- | AfAfCfCfAmGmUmUmGmAmAps | | fUmGmGmUmUfUmGfUmAmG | |
| Chol | (InvAb)(Chol4) | | mAmCmUmAmUpsmUpsmU | |
| M25_V | (InvAb)psmApsmGmUmCmUmA | 33 | (VPmU)psfUpsmCpsmAmA | 34 |
| ar6- | fCfAfAmAmCmCmAmGmUmUmGm | | fCmUmGmGmUmUmUmGfUmA | |
| Chol | AmCpsmCpsmA(Chol4) | | fGmAmCmUmApsmU | |
| M25_V | (InvAb)psmApsmUmAmGmUmC | 35 | (VPmU)psfUpsmCpsfAmA | 36 |
| ar7- | mUmAfCfAfAmAmCmCmAmGmUm | | fCmUfGmGfUmUfUmGfUmA | |
| Chol | UmGmApsmAps(InvAb) | | fGmAfCmUmApsmU | |
| | (Chol4) | | | |
| M25_V | (InvAb)psmApsmGmUmCmUmA | 37 | (VPmU)psfUpsmCmAmAfC | 38 |
| ar8- | fCfAfAmAmCmCmAmGmUmUmGm | | mUfGfGmUmUmUmGfUmAfG | |
| Chol | ApsmAps(InvAb)(Chol4) | | mAmCmUmAmUpsmUpsmU | |
| M25_V | mApsmUpsmAmGmUmCfUmAfCf | 39 | (VPmU)psfUpsmCpsfAmA | 40 |
| ar9- | AfAmAmCmCmAmGmUmUmGpsmA | | fCmUfGmGfUmUfUmGfUmA | |
| Chol | psmA(Chol4) | | fGmAfCmUmApsmU | |
| M25_V | (InvAb)psmApsmGmUmCmUmA | 41 | (VPmU)psfUpsmCpsmAmA | 42 |
| ar10- | fCfAfAmAmCmCmAmGmUmUmGm | | fCmUfGfGmUmUmUmGfUmA | |
| Chol | ApsmAps(InvAb)(Chol4) | | fGmAmCmUpsmApsmU | |
| M25_V | mApsmGpsmUmCfUmAfCfAfAm | 43 | (VPmU)psfUpsmCpsfAmA | 44 |
| ar11- | AmCmCmAmGmUmUmGpsmApsmA | | fCmUfGmGfUmUfUmGfUmA | |
| Chol | (Chol4) | | fGmAfCmUmApsmU | |
| M25_V | (InvAb)psmApsmGmUmCmUmA | 45 | (VPmU)psfUpsmCmAmAmC | 46 |
| ar12- | fCfAfAmAmCmCmAmGmUmUmGm | | fUmGmGmUmUfUmGfUmAmG | |
| Chol | ApsmAps(InvAb)(Chol4) | | mAmCmUmAmUpsmUpsmU | |
| M25_V | mUpsmApsmGmUmCmUmAmCfAm | 47 | (VPmU)psfUpsmCpsfAmA | 48 |
| ar13- | AfAfCfCfAmGmUmUmGmAmAps | | fCmUfGmGfUmUfUmGfUmA | |
| Chol | (InvAb)(Chol4) | | fGmAfCmUmApsmU | |
| M25_V | (InvAb)psmApsmUmAmGmUmC | 49 | (VPmU)psfUpsmCmAmAfC | 50 |
| ar14- | mUmAfCfAfAmAmCmCmAmGmUm | | mUfGfGmUmUmUmGfUmAfG | |
| Chol | UmGmApsmAps(InvAb) | | mAmCmUmAmUpsmUpsmU | |
| | (Chol4) | | | |
| M25_V | (InvAb)psmApsmUmAmGmUmC | 51 | (VPmU)psfUpsmCpsmAmA | 52 |
| ar15- | mUmAfCfAfAmAmCmCmAmGmUm | | fCmUfGfGmUmUmUmGfUmA | |
| Chol | UmGmApsmAps(InvAb) | | fGmAmCmUpsmApsmU | |
| | (Chol4) | | | |
| M25_V | (InvAb)psmApsmUmAmGmUmC | 53 | (VPmU)psfUpsmCmAmAmC | 54 |
| ar16- | mUmAfCfAfAmAmCmCmAmGmUm | | fUmGmGmUmUfUmGfUmAmG | |
| Chol | UmGmApsmAps(InvAb) | | mAmCmUmAmUpsmUpsmU | |
| | (Chol4) | | | |
| M25_V | mApsmUpsmAmGmUmCfUmAfCf | 55 | (VPmU)psfUpsmCmAmAfC | 56 |
| ar17- | AfAmAmCmCmAmGmUmUmGpsmA | | mUmGfGmUmUmUmGfUmAfG | |
| Chol | psmA(Chol4) | | mAmCmUmAmUpsmUpsmU | |
| M25_V | mApsmUpsmAmGmUmCfUmAfCf | 57 | (VPmU)psfUpsmCmAmAfC | 58 |
| ar18- | AfAmAmCmCmAmGmUmUmGpsmA | | mUfGmGmUmUmUmGfUmAfG | |
| Chol | psmA(Chol4) | | mAmCmUmAmUpsmUpsmU | |
| M25_V | mApsmUpsmAmGmUmCfUmAfCf | 59 | (VPmU)psfUpsmCmAmAfC | 60 |
| ar19- | AfAmAmCmCmAmGmUmUmGpsmA | | mUmGmGmUmUmUmGfUmAfG | |
| Chol | psmA(Chol4) | | mAmCmUmAmUpsmUpsmU | |
| M25_V | (InvAb)psmApsmUmAmGmUmC | 61 | (VPmU)psfUpsmCmAmAmC | 62 |
| ar20- | mUmAfCfAfAmAmCmCmAmGmUm | | (UNAU)mGmGmUmUmUmGfU | |
| Chol | UmGmApsmAps(InvAb) | | mAfGmAmCmUpsmApsmU | |
| | (Chol4) | | | |
| M25_V | (InvAb)psmApsmGmUmCmUmA | 63 | (VPmU)psfUpsmCmAmAmC | 64 |
| ar21- | fCfAfAmAmCmCmAmGmUmUmGm | | (UNAU)mGmGmUmUmUmGfU | |
| Chol | ApsmAps(InvAb)(Chol4) | | mAfGmAmCmUmAmUpsmUps | |
| | | | mU | |
| M25_V | (InvAb)psmApsmUmAmGmUmC | 65 | (VPmU)psfUpsmCmAmAfC | 66 |
| ar22- | mUmAfCfAdAmAmCmCmAmGmUm | | mUfGfGmUmUmUmGfUmAfG | |
| Chol | UmGmApsmAps(InvAb) | | mAmCmUpsmApsmU | |
| | (Chol4) | | | |
| M25_V | (InvAb)psmApsmUmAmGmUmC | 67 | (VPmU)psfUpsmCpsmAmA | 68 |
| ar23- | mUmAfCfAfAmAmCmCmAmGmUm | | fCmUfGfGmUmUmUmGfUmA | |
| Chol | UmGmApsmAps(InvAb) | | fGmAmCmUmApsmU | |
| | (Chol4) | | | |
| M25_V | mApsmGpsfUmCmUmAfCfAfAm | 69 | (VPmU)psfUpsmCpsmAmA | 70 |
| ar24- | AmCfCmAmGmUmUfGpsmApsmA | | fC(UNA-U)mGmGmUmUmUm | |
| Chol | (Chol4) | | GfUmAfGmAmCmUmApsmU | |
| M25_V | (InvAb)psmApsmGfUmCmUmA | 71 | (VPmU)psfUpsmCmAmAfC | 72 |
| ar25- | fCfAfAmAmCfCmAmGmUmUfGp | | (UNA-U)mGmGmUmUmUmGf | |
| Chol | smApsmA(Chol4) | | UmAfGmAmCmUpsmApsmU | |
| M25_V | (InvAb)psmApsmGfUmCmUmA | 73 | (VPmU)psfUpsmCpsmAmA | 74 |
| ar26- | fCfAfAmAmCfCmAmGmUmUfGp | | fC(UNA-U)mGmGmUmUmUm | |
| Chol | smApsmA(Chol4) | | GfUmAfGmAmCmUmApsmU | |
| M25_V | (InvAb)psmApsmGmUmCmUmA | 75 | (VPmU)psfUpsmCmAmAfC | 76 |
| ar27- | fCfAfAmAmCmCmAmGmUmUmGm | | mUmGfGmUmUmUmGfUmAfG | |
| Chol | ApsmAps(InvAb)(Chol4) | | mAmCmUpsmApsmU | |
| M25_V | (InvAb)psmApsmUmAmGmUmC | 77 | (VPmU)psfUpsmCmAmAmC | 78 |
| ar28- | mUmAfCfAfAmAmCmCmAmGmUm | | (UNA-U)mGmGmUmUmUmGf | |
| Chol | UmGmAmAps(InvAb)ps | | UmAfGmAmCmUpsmApsmU | |
| | (Chol4) | | | |
| M25_V | (InvAb)psmApsmUmAmGmUmC | 79 | (VPmU)psfUpsmCmAmAfC | 80 |
| ar29- | mUmAfCfAfAmAmCmCmAmGmUm | | (UNA-U)mGmGmUmUmUmGf | |
| Chol | UmGmAmAps(InvAb)ps | | UmAfGmAmCmUpsmApsmU | |
| | (Chol4) | | | |
| M25_V | (InvAb)psmApsmUmAmGmUmC | 81 | (VPmU)psfUpsmCmAmAmC | 82 |
| ar30- | mUmAfCfAfAmAmCmCmAmGmUm | | (UNAU)fGmGmUmUmUmGfU | |
| Chol | UmGmAmAps(InvAb)ps | | mAfGmAmCmUpsmApsmU | |
| | (Chol4) | | | |
| M25_V | (InvAb)psmApsmUmAmGmUmC | 83 | (VPmU)psfUpsmCmAmAmC | 84 |
| ar31- | mUmAfCfAfAmAmCmCmAmGmUm | | (UNAU)mGfGmUmUmUmGfU | |
| Chol | UmGmAmAps(InvAb)ps | | mAfGmAmCmUpsmApsmU | |
| | (Chol4) | | | |
| M25_V | (InvAb)psmApsmUmAmGmUmC | 85 | (VPmU)psfUpsmCmAmAfC | 86 |
| ar32- | mUmAfCfAfAmAmCmCmAmGmUm | | (UNA-U)mGfGmUmUmUmGf | |
| Chol | UmGmAmAps(InvAb)ps | | UmAfGmAmCmUpsmApsmU | |
| | (Chol4) | | | |
| M25_V | (InvAb)psmApsmUmAmGmUmC | 87 | (VPmU)psfUpsmCmAmAmC | 88 |
| ar33- | mUmAfCfAfAmAmCmCmAmGmUm | | (GNAT)mGmGmUmUmUmGfU | |
| Chol | UmGmAmAps(InvAb)ps | | mAfGmAmCmUpsmApsmU | |
| | (Chol4) | | | |
| M25_V | (InvAb)psmApsmUmAmGmUmC | 89 | (VPmU)psfUpsmCmAmAmC | 90 |
| ar34- | mUmAfCfAfAmAmCmCmAmGmUm | | dTmGmGmUmUmUmGfUmAfG | |
| Chol | UmGmAmAps(InvAb)ps | | mAmCmUpsmApsmU | |
| | (Chol4) | | | |
| M25_V | (InvAb)psmApsmUmAmGmUmC | 91 | (VPmU)psfUps(UNA-C)m | 92 |
| ar35- | mUmAfCfAfAmAmCmCmAmGmUm | | AmAmCmUmGmGmUmUmUmGf | |
| Chol | UmGmAmAps(InvAb)ps | | UmAfGmAmCmUpsmApsmU | |
| | (Chol4) | | | |
| M25_V | (InvAb)psmApsmUmAmGmUmC | 93 | (VPmU)psfUpsmC(UNA- | 94 |
| ar36- | mUmAfCfAfAmAmCmCmAmGmUm | | A)mAmCmUmGmGmUmUmUmG | |
| Chol | UmGmAmAps(InvAb)ps | | fUmAfGmAmCmUpsmApsmU | |
| | (Chol4) | | | |
| M25_V | (InvAb)psmApsmUmAmGmUmC | 95 | (VPmU)psfUpsmCmA | 96 |
| ar37- | mUmAfCfAfAmAmCmCmAmGmUm | | (UNA-A)mCmUmGmGmUmUm | |
| Chol | UmGmAmAps(InvAb)ps | | UmGfUmAfGmAmCmUpsmAp | |
| | (Chol4) | | smU | |
| M25_V | (InvAb)psmApsmUmAmGmUmC | 97 | (VPmU)psfUpsmCmAmA | 98 |
| ar38- | mUmAfCfAfAmAmCmCmAmGmUm | | (UNA-C)mUmGmGmUmUmUm | |
| Chol | UmGmAmAps(InvAb)ps | | GfUmAfGmAmCmUpsmApsm | |
| | (Chol4) | | U | |
| M25_V | (InvAb)psmApsmUmAmGmUmC | 99 | (VPmU)psfUpsmCmAmAfC | 100 |
| ar39- | mUmAfCfAfAmAmCmCmAmGmUm | | mUfGfGmUmUmUdGfUmAfG | |
| Chol | UmGmAmAps(InvAb)ps | | mAmCmUpsmApsmU | |
| | (Chol4) | | | |
| M25_V | (InvAb)psmApsmUmAmGmUmC | 101 | (VPmU)psfUpsmCmAmAfC | 102 |
| ar40- | mUmAfCfAfAmAmCmCmAmGmUm | | mUfGfGmUmUmUmGdTmAfG | |
| Chol | UmGmAmAps(InvAb)ps | | mAmCmUpsmApsmU | |
| | (Chol4) | | | |
| M25_V | (InvAb)psmApsmUmAmGmUmC | 103 | (VPmU)psfUpsmCmAmAfC | 104 |
| ar41- | mUmAfCfAfAmAmCmCmAmGmUm | | mUfGfGmUmUmUmGfUdAfG | |
| Chol | UmGmAmAps(InvAb)ps | | mAmCmUpsmApsmU | |
| | (Chol4) | | | |
| M25_V | (InvAb)psmApsmUmAmGmUmC | 105 | (VPmU)psfUpsmCmAmAfC | 106 |
| ar42- | mUmAfCfAfAmAmCmCmAmGmUm | | mUfGfGmUmUmUmGfUmAdG | |
| Chol | UmGmAmAps(InvAb)ps | | mAmCmUpsmApsmU | |
| | (Chol4) | | | |
| M25_V | (InvAb)psmApsmGmUmCmUmA | 107 | (VPmU)psfUpsmCmAmAfC | 108 |
| ar43- | fCfAfAmAmCfCmAmGmUmUfGm | | (UNA-U)mGmGmUmUmUmGf | |
| Chol | ApsmAps(Chol4) | | UmAfGmAmCmUpsmApsmU | |
| M25_V | (InvAb)psmApsmGfUmCmUmA | 109 | (VPmU)psfUpsmCmAmAfC | 110 |
| ar44- | fCfAfAmAmCfCmAmGmUmUmGm | | (UNA-U)mGmGmUmUmUmGf | |
| Chol | ApsmAps(Chol4) | | UmAfGmAmCmUpsmApsmU | |
| M25_V | (InvAb)psmApsmGmUmCmUmA | 111 | (VPmU)psfUpsmCmAmAfC | 112 |
| ar45- | fCfAfAmAmCfCmAmGmUmUmGm | | (UNA-U)mGmGmUmUmUmGf | |
| Chol | ApsmAps(Chol4) | | UmAfGmAmCmUpsmApsmU | |
| M28- | mApsmApsfGmUfGmUfGmGfCm | 113 | (VPmU)psfGpsmCfCmUfA | 114 |
| Chol | UfCmAfUmUfAmGfGmCfApsmU | | mAfUmGfAmGfCmCfAmCfA | |
| | psmU(Chol4) | | mCfUmUpsmUpsmU | |
| M28_V | mApsmApsfGmUmGmUfGfGfCm | 115 | (VPmU)psfGpsmCmCmUfA | 116 |
| ar1- | UmCfAmUmUmAmGfGmCmA | | mAmUmGmAmGmCmCfAmCfA | |
| Chol | (Chol4) | | mCmUmUpsmGpsmG | |
| M28_V | mApsmApsfGmUmGmUfGfGfCm | 117 | (VPmU)psfGpsmCmCmUfA | 118 |
| ar2- | UmCfAmUmUmAmGfGmCmA | | (GNA-A)mUmGmAmGmCmCf | |
| Chol | (Chol4) | | AmCfAmCmUmUpsmGpsmG | |
| M28_V | (InvAb)psmApsmAmGmUmGmU | 119 | (VPmU)psfGpsmCpsfCmU | 120 |
| ar3- | fGfGfCmUmCmAmUmUmAmGmGm | | fAmAfUmGfAmGfCmCfAmC | |
| Chol | CpsmAps(InvAb)(Chol4) | | fAmCfUmUmGpsmG | |
| M28_V | mApsmApsmGmUfGmUfGfGfCm | 121 | (VPmU)psfGpsmCmCmUfA | 122 |
| ar4- | UmCmAmUmUmAmGmGpsmCpsmA | | mAfUfGmAmGmCmCfAmCfA | |
| Chol | (Chol4) | | mCmUmUpsmGpsmG | |
| M28_V | mCpsmCpsmAmAmGmUfGmUfGf | 123 | (VPmU)psfGpsmCmCmUfA | 124 |
| ar5- | GfCmUmCmAmUmUmAmGmGpsmC | | mAfUfGmAmGmCmCfAmCfA | |
| Chol | psmA(Chol4) | | mCmUmUmGmGpsmApsmG | |
| M28_V | mCpsmApsmAmGmUmGmUmGfGm | 125 | (VPmU)psfGpsmCmCmUmA | 126 |
| ar6- | CfUfCfAfUmUmAmGmGmCmAps | | fAmUmGmAmGfCmCfAmCmA | |
| Chol | (InvAb)(Chol4) | | mCmUmUmGmGpsmUpsmU | |
| M28_V | (InvAb)psmApsmAmGmUfGmU | 127 | (VPmU)psfGpsmCmCmUfA | 128 |
| ar7- | fGfGfCmUmCmAmUmUmAmGmGm | | mAmUmGmAmGmCmCfAmCfA | |
| Chol | CmApsmApsmC(Chol4) | | mCmUmUpsmGpsmG | |
| M28_V | (InvAb)psmCpsmCmAmAmGmU | 129 | (VPmU)psfGpsmCpsfCmU | 130 |
| ar8- | mGmUfGfGfCmUmCmAmUmUmAm | | fAmAfUmGfAmGfCmCfAmC | |
| Chol | GmGmCpsmAps(InvAb) | | fAmCfUmUmGpsmG | |
| | (Chol4) | | | |
| M28_V | (InvAb)psmApsmAmGmUmGmU | 131 | (VPmU)psfGpsmCmCmUfA | 132 |
| ar9- | fGfGfCmUmCmAmUmUmAmGmGm | | mAfUfGmAmGmCmCfAmCfA | |
| Chol | CpsmAps(InvAb)(Chol4) | | mCmUmUmGmGpsmApsmG | |
| M28_V | mCpsmCpsmAmAmGmUfGmUfGf | 133 | (VPmU)psfGpsmCpsfCmU | 134 |
| ar10- | GfCmUmCmAmUmUmAmGmGpsmC | | fAmAfUmGfAmGfCmCfAmC | |
| Chol | psmA(Chol4) | | fAmCfUmUmGpsmG | |
| M28_V | (InvAb)psmApsmAmGmUmGmU | 135 | (VPmU)psfGpsmCmCmUfA | 136 |
| ar11- | fGfGfCmUmCmAmUmUmAmGmGm | | mAfUfGmAmGmCmCfAmCfA | |
| Chol | CpsmAps(InvAb)(Chol4) | | mCmUmUpsmGpsmG | |
| M28_V | mApsmApsmGmUfGmUfGfGfCm | 137 | (VPmU)psfGpsmCpsfCmU | 138 |
| ar12- | UmCmAmUmUmAmGmGpsmCpsmA | | fAmAfUmGfAmGfCmCfAmC | |
| Chol | (Chol4) | | fAmCfUmUmGpsmG | |
| M28_V | (InvAb)psmApsmAmGmUmGmU | 139 | (VPmU)psfGpsmCmCmUmA | 140 |
| ar13- | fGfGfCmUmCmAmUmUmAmGmGm | | fAmUmGmAmGfCmCfAmCmA | |
| Chol | CpsmAps(InvAb)(Chol4) | | mCmUmUmGmGpsmUpsmU | |
| M28_V | mCpsmApsmAmGmUmGmUmGfGm | 141 | (VPmU)psfGpsmCpsfCmU | 142 |
| ar14- | CfUfCfAfUmUmAmGmGmCmAps | | fAmAfUmGfAmGfCmCfAmC | |
| Chol | (InvAb)(Chol4) | | fAmCfUmUmGpsmG | |
| M28_V | (InvAb)psmCpsmCmAmAmGmU | 143 | (VPmU)psfGpsmCmCmUfA | 144 |
| ar15- | mGmUfGfGfCmUmCmAmUmUmAm | | mAfUfGmAmGmCmCfAmCfA | |
| Chol | GmGmCpsmAps(InvAb) | | mCmUmUmGmGpsmApsmG | |
| | (Chol4) | | | |
| M28_V | (InvAb)psmCpsmCmAmAmGmU | 145 | (VPmU)psfGpsmCmCmUfA | 146 |
| ar16- | mGmUfGfGfCmUmCmAmUmUmAm | | mAfUfGmAmGmCmCfAmCfA | |
| Chol | GmGmCpsmAps(InvAb) | | mCmUmUpsmGpsmG | |
| | (Chol4) | | | |
| M28_V | (InvAb)psmCpsmCmAmAmGmU | 147 | (VPmU)psfGpsmCmCmUmA | 148 |
| ar17- | mGmUfGfGfCmUmCmAmUmUmAm | | fAmUmGmAmGfCmCfAmCmA | |
| Chol | GmGmCpsmAps(InvAb) | | mCmUmUmGmGpsmUpsmU | |
| | (Chol4) | | | |
| M28_V | mCpsmCpsmAmAmGmUfGmUfGf | 149 | (VPmU)psfGpsmCmCmUfA | 150 |
| ar18- | GfCmUmCmAmUmUmAmGmGpsmC | | mAmUfGmAmGmCmCfAmCfA | |
| Chol | psmA(Chol4) | | mCmUmUmGmGpsmApsmG | |
| M28_V | mCpsmCpsmAmAmGmUfGmUfGf | 151 | (VPmU)psfGpsmCmCmUfA | 152 |
| ar19- | GfCmUmCmAmUmUmAmGmGpsmC | | mAfUmGmAmGmCmCfAmCfA | |
| Chol | psmA(Chol4) | | mCmUmUmGmGpsmApsmG | |
| M28_V | mCpsmCpsmAmAmGmUfGmUfGf | 153 | (VPmU)psfGpsmCmCmUfA | 154 |
| ar20- | GfCmUmCmAmUmUmAmGmGpsmC | | mAmUmGmAmGmCmCfAmCfA | |
| Chol | psmA(Chol4) | | mCmUmUmGmGpsmApsmG | |
| M28_V | mApsmApsmGmUfGmUfGfGfCm | 165 | (VPmU)psfGpsmCmCmUfA | 166 |
| ar26- | UmCmAmUmUmAmGmGpsmCpsmA | | mAmUmGmAmGmCmCfAmCfA | |
| Chol | (Chol4) | | mCmUmUpsmGpsmG | |

Modification:
mN = 2′OMe;
fN = 2′F;
ps = phosphorothioate;
VP = vinyl phosphonate;
invAb = inverse abasic

TABLE 5
Modified Sense and Antisense Strand Nucleotide
Sequences (5′ to 3′)

| | | SEQ ID | | SEQ ID |
|---|---|---|---|---|
| NAME | Sense strand | NO | Antisense strand | NO |
| M28_V | (L2)mApsmApsfGmUmGmUfGf | 179 | (VPmU)psfGpsmCmCmUfAmAm | 180 |
| ar29 | GfCmUmCfAmUmUmAmGfGpsmC | | UmGmAmGmCmCfAmCfAmCmUmU | |
| | psmA | | psmGpsmG | |
| M28_V | mApsmApsfGmUmGmUfGfGfCm | 181 | (VPmU)psfGpsmCmCmUfAmAm | 182 |
| ar30 | UmCfAmUmUmAmGfGpsmCpsmA | | UmGmAmGmCmCfAmCfAmCmUmU | |
| | dTdT(L2) | | psmGpsmG | |
| M28_V | (L2)mApsmApsfGmUmGmUfGf | 187 | (VPmU)psfGpsmCmCmUfA | 188 |
| ar33 | GfCmUmCfAmUmUmAmGfGpsmC | | (GNA-A)mUmGmAmGmCmCfAmC | |
| | psmA | | fAmCmUmUpsmGpsmG | |
| M28_V | mApsmApsfGmUmGmUfGfGfCm | 189 | (VPmU)psfGpsmCmCmUfA | 190 |
| ar34 | UmCfAmUmUmAmGfGpsmCpsmA | | (GNA-A)mUmGmAmGmCmCfAmC | |
| | dTdT(L2) | | fAmCmUmUpsmGpsmG | |
| M28_V | mApsmApsfGmUmGmUfGfGfCm | 191 | (VPmU)psfGpsmCmCmUfAmAm | 192 |
| ar35 | UmCfAmUmUmAmGfGpsmCpsmA | | UmGmAmGmCmCfAmCfAmCmUmU | |
| | dTdT(Chol4) | | psmGpsmG | |
| M28_V | (Chol4)mApsmApsfGmUmGmU | 193 | (VPmU)psfGpsmCmCmUfAmAm | 194 |
| ar36 | fGfGfCmUmCfAmUmUmAmGfGp | | UmGmAmGmCmCfAmCfAmCmUmU | |
| | smCpsmA | | psmGpsmG | |
| M28_V | mApsmApsfGmUmG(L1)fGfGf | 195 | (VPmU)psfGpsmCmCmUfAmAm | 196 |
| ar37 | CmUmCfAmUmUmAmGfGpsmCps | | UmGmAmGmCmCfAmCfAmCmUmU | |
| | mA | | psmGpsmG | |
| M28_V | mApsmApsfGmUmG(L3)fGfGf | 197 | (VPmU)psfGpsmCmCmUfAmAm | 198 |
| ar38 | CmUmCfAmUmUmAmGfGpsmCps | | UmGmAmGmCmCfAmCfAmCmUmU | |
| | mA | | psmGpsmG | |
| M28_V | mApsmApsfGmUmG(L4)fGfGf | 199 | (VPmU)psfGpsmCmCmUfAmAm | 200 |
| ar39 | CmUmCfAmUmUmAmGfGpsmCps | | UmGmAmGmCmCfAmCfAmCmUmU | |
| | mA | | psmGpsmG | |
| M28_V | mApsmApsfGmUmG(L7)fGfGf | 201 | (VPmU)psfGpsmCmCmUfAmAm | 202 |
| ar40 | CmUmCfAmUmUmAmGfGpsmCps | | UmGmAmGmCmCfAmCfAmCmUmU | |
| | mA | | psmGpsmG | |
| M28_V | mApsmApsfGmUmG(L5)fGfGf | 203 | (VPmU)psfGpsmCmCmUfAmAm | 204 |
| ar41 | CmUmCfAmUmUmAmGfGpsmCps | | UmGmAmGmCmCfAmCfAmCmUmU | |
| | mA | | psmGpsmG | |
| M28_V | mApsmApsfGmUmG(L6)fGfGf | 205 | (VPmU)psfGpsmCmCmUfAmAm | 206 |
| ar42 | CmUmCfAmUmUmAmGfGpsmCps | | UmGmAmGmCmCfAmCfAmCmUmU | |
| | mA | | psmGpsmG | |
| M28_V | mApsmApsfGmUmGmUfGfGfCm | 207 | (VPmU)psfGpsmCmCmUfAmAm | 208 |
| ar43 | UmCfAmUmUmAmGfGpsmCpsmA | | UmGmAmGmCmCfAmCfAmCmUmU | |
| | dTdT(L10) | | psmGpsmG | |
| M28_V | mApsmApsfGmUmGmUfGfGfCm | 209 | (VPmU)psfGpsmCmCmUfAmAm | 210 |
| ar44 | UmCfAmUmUmAmGfGpsmCpsmA | | UmGmAmGmCmCfAmCfAmCmUmU | |
| | (L9) | | psmGpsmG | |
| M28_V | (L9)mApsmApsfGmUmGmUfGf | 211 | (VPmU)psfGpsmCmCmUfAmAm | 212 |
| ar45 | GfCmUmCfAmUmUmAmGfGpsmC | | UmGmAmGmCmCfAmCfAmCmUmU | |
| | psmA | | psmGpsmG | |
| M28_V | mApsmApsfGmUmGmUfGfGfCm | 213 | (VPmU)psfGpsmCmCmUfAmAm | 214 |
| ar46 | UmCfAmUmUmAmGfGpsmCpsmA | | UmGmAmGmCmCfAmCfAmCmUmU | |
| | (L8) | | psmGpsmG | |
| M28_V | (L9)mApsmApsfGmUmGmUfGf | 215 | (VPmU)psfGpsmCmCmUfAmAm | 216 |
| ar47 | GfCmUmCfAmUmUmAmGfGpsmC | | UmGmAmGmCmCfAmCfAmCmU | |
| | psmA(L9) | | mUpsmGpsmG | |
| M28_V | (L8)mApsmApsfGmUmGmUfGf | 217 | (VPmU)psfGpsmCmCmUfAmAm | 218 |
| ar48 | GfCmUmCfAmUmUmAmGfGpsmC | | UmGmAmGmCmCfAmCfAmCmUmU | |
| | psmA(L8) | | psmGpsmG | |
| M28_V | mApsmApsfGmUmGmUfGfGfCm | 219 | (VPmU)psfGpsmCmCmUfAmAm | 220 |
| ar49 | UmCfAmUmUmAmGfGpsmCpsmA | | UmGmAmGmCmCfAmCfAmCmUmU | |
| | (C7NH-L17) | | psmGpsmG | |
| M25_V | (InvAb)psmApsmUmAmGmUmC | 221 | (VPmU)psfUpsmCpsmAmAfCm | 222 |
| ar46 | mUmAfCfAfAmAmCmCmAmGmUm | | UfGfGmUmUmUmGfUmAfGmAmC | |
| | UmGmApsmAps(InvAb) | | mUpsmApsmU | |
| | (C7NH-L20) | | | |
| M25_V | (InvAb)psmApsmUmAmGmUmC | 223 | (VPmU)psfUpsmCpsmAmAfCm | 224 |
| ar47 | mUmAfCfAfAmAmCmCmAmGmUm | | UfGfGmUmUmUmGfUmAfGmAmC | |
| | UmGmApsmAps(InvAb) | | mUpsmApsmU | |
| | (C7NH-L18) | | | |
| M25_V | (InvAb)psmApsmUmAmGmUmC | 225 | (VPmU)psfUpsmCpsmAmAfCm | 226 |
| ar48 | mUmAfCfAfAmAmCmCmAmGmUm | | UfGfGmUmUmUmGfUmAfGmAmC | |
| | UmGmApsmAps(InvAb) | | mUpsmApsmU | |
| | (C7NH-L16) | | | |
| M25_V | (InvAb)psmApsmUmAmGmUmC | 227 | (VPmU)psfUpsmCpsmAmAfCm | 228 |
| ar49 | mUmAfCfAfAmAmCmCmAmGmUm | | UfGfGmUmUmUmGfUmAfGmAmC | |
| | UmGmApsmAps(InvAb) | | mUpsmApsmU | |
| | (C7NH-L17) | | | |
| M25_V | (InvAb)psmApsmUmAmGmUmC | 229 | (VPmU)psfUpsmCpsmAmAfCm | 230 |
| ar50 | mUmAfCfAfAmAmCmCmAmGmUm | | UfGfGmUmUmUmGfUmAfGmAmC | |
| | UmGmApsmAps(InvAb)(L12) | | mUpsmApsmU | |
| M25_V | (InvAb)psmApsmUmAmGmUmC | 231 | (VPmU)psfUpsmCpsmAmAfCm | 232 |
| ar51 | mUmAfCfAfAmAmCmCmAmGmUm | | UfGfGmUmUmUmGfUmAfGmAmC | |
| | UmGmApsmAps(InvAb)(L11) | | mUpsmApsmU | |
| M25_V | (InvAb)psmApsmUmAmGmUmC | 233 | (VPmU)psfUpsmCpsmAmAfCm | 234 |
| ar52 | mUmAfCfAfAmAmCmCmAmGmUm | | UfGfGmUmUmUmGfUmAfGmAmC | |
| | UmGmApsmAps(InvAb)(L24) | | mUpsmApsmU | |
| M25_V | (InvAb)psmApsmUmAmGmUmC | 235 | (VPmU)psfUpsmCpsmAmAfCm | 236 |
| ar53 | (C6NH-L15U)mAfCfAfAmAmC | | UfGfGmUmUmUmGfUmAfGmAmC | |
| | mCmAmGmUmUmGmApsmAps | | mUpsmApsmU | |
| | (InvAb) | | | |
| M25_V | (InvAb)psmApsmUmAmGmUmC | 237 | (VPmU)psfUpsmCpsmAmAfCm | 238 |
| ar54 | (C6NH-L18U)mAfCfAfAmAmC | | UfGfGmUmUmUmGfUmAfGmAmC | |
| | mCmAmGmUmUmGmApsmAps | | mUpsmApsmU | |
| | (InvAb) | | | |
| M25_V | (InvAb)psmApsmUmAmGmUmC | 239 | (VPmU)psfUpsmCpsmAmAfCm | 240 |
| ar55 | (C6NH-L19U)mAfCfAfAmAmC | | UfGfGmUmUmUmGfUmAfGmAmC | |
| | mCmAmGmUmUmGmApsmAps | | mUpsmApsmU | |
| | (InvAb) | | | |
| M25_V | (InvAb)psmApsmUmAmGmUmC | 241 | (VPmU)psfUpsmCpsmAmAfCm | 242 |
| ar56 | (C6NH-L17U)mAfCfAfAmAmC | | UfGfGmUmUmUmGfUmAfGmAmC | |
| | mCmAmGmUmUmGmApsmAps | | mUpsmApsmU | |
| | (InvAb) | | | |
| M25_V | (InvAb)psmApsmUmAmGmUmC | 243 | (VPmU)psfUpsmCpsmAmAfCm | 244 |
| ar57 | (C6NH-L4U)mAfCfAfAmAmCm | | UfGfGmUmUmUmGfUmAfGmAmC | |
| | CmAmGmUmUmGmApsmAps | | mUpsmApsmU | |
| | (InvAb) | | | |
| M25_V | (InvAb)psmApsmUmAmGmUmC | 247 | (VPmU)psfUpsmCpsmAmAfCm | 248 |
| ar59 | (C6NH-L16U)mAfCfAfAmAmC | | UfGfGmUmUmUmGfUmAfGmAmC | |
| | mCmAmGmUmUmGmApsmAps | | mUpsmApsmU | |
| | (InvAb) | | | |
| M25_V | (InvAb)psmApsmUmAmGmUmC | 249 | (VPmU)psfUpsmCpsmAmAfCm | 250 |
| ar60 | (C6NH-L20U)mAfCfAfAmAmC | | UfGfGmUmUmUmGfUmAfGmAmC | |
| | mCmAmGmUmUmGmApsmAps | | mUpsmApsmU | |
| | (InvAb) | | | |
| M25_V | (InvAb)psmApsmUmAmGmUmC | 253 | (VPmU)psfUpsmCpsmAmAfCm | 254 |
| ar62 | mUmAfCfAfAmAmCmCmAmGmUm | | UfGfGmUmUmUmGfUmAfGmAmC | |
| | UmGmApsmAps(InvAb) | | mUpsmApsmU | |
| | (C7NH-L14) | | | |
| M25_V | (InvAb)psmApsmUmAmGmUmC | 255 | (VPmU)psfUpsmCpsmAmAfCm | 256 |
| ar63 | mUmAfCfAfAmAmCmCmAmGmUm | | UfGfGmUmUmUmGfUmAfGmAmC | |
| | UmGmApsmAps(InvAb) | | mUpsmApsmU | |
| | (C7NH-L15) | | | |
| M25_V | (InvAb)psmApsmUmAmGmUmC | 257 | (VPmU)psfUpsmCpsmAmAfCm | 258 |
| ar64 | mUmAfCfAfAmAmCmCmAmGmUm | | UfGfGmUmUmUmGfUmAfGmAmC | |
| | UmGmApsmAps(InvAb) | | mUpsmApsmU | |
| | (C7NH-L19) | | | |
| M25_V | (InvAb)psmApsmUmAmGmUmC | 261 | (VPmU)psfUpsmCmAmAfCmUf | 262 |
| ar66 | (L13)mAfCfAfAmAmCmCmAmG | | GfGmUmUmUmGfUmAfGmAmCm | |
| | mUmUmGmApsmAps(InvAb) | | UpsmApsmU | |
| M25_V | (InvAb)psmApsmUmAmGmUmC | 263 | (VPmU)psfUpsmCpsmAmAfCm | 264 |
| ar68 | (L4)mAfCfAfAmAmCmCmAmGm | | UfGfGmUmUmUmGfUmAfGmAmC | |
| | UmUmGmApsmAps(InvAb) | | mUpsmApsmU | |
| M25_V | (InvAb)psmAps(L5)mAmGmU | 265 | (VPmU)psfUpsmCpsmAmAfCm | 266 |
| ar73 | mCmUmAfCfAfAmAmCmCmAmGm | | UfGfGmUmUmUmGfUmAfGmAmC | |
| | UmUmGmApsmAps(InvAb) | | mUpsmApsmU | |
| M25_V | (InvAb)psmApsmUmAmG(L5) | 267 | (VPmU)psfUpsmCpsmAmAfCm | 268 |
| ar74 | mCmUmAfCfAfAmAmCmCmAmGm | | UfGfGmUmUmUmGfUmAfGmAmC | |
| | UmUmGmApsmAps(InvAb) | | mUpsmApsmU | |
| M25_V | (InvAb)psmApsmUmAmGmUmC | 269 | (VPmU)psfUpsmCpsmAmAfCm | 270 |
| ar75 | (L5)mAfCfAfAmAmCmCmAmGm | | UfGfGmUmUmUmGfUmAfGmAmC | |
| | UmUmGmApsmAps(InvAb) | | mUpsmApsmU | |
| M25_V | (InvAb)psmApsmUmAmGmUmC | 271 | (VPmU)psfUpsmCpsmAmAfCm | 272 |
| ar76 | mUmAfCfAfAmAmCmCmAmG | | UfGfGmUmUmUmGfUmAfGmAmC | |
| | (L5)mUmGmApsmAps(InvAb) | | mUpsmApsmU | |
| M25_V | (InvAb)psmApsmUmAmGmUmC | 273 | (VPmU)psfUpsmCpsmAmAfCm | 274 |
| ar77 | mUmAfCfAfAmAmCmCmAmGmU | | UfGfGmUmUmUmGfUmAfGmAmC | |
| | (L5)mGmApsmAps(InvAb) | | mUpsmApsmU | |
| M25_V | (InvAb)psmAps(L4)mAmGmU | 275 | (VPmU)psfUpsmCpsmAmAfCm | 276 |
| ar78 | mCmUmAfCfAfAmAmCmCmAmGm | | UfGfGmUmUmUmGfUmAfGmAmC | |
| | UmUmGmApsmAps(InvAb) | | mUpsmApsmU | |
| M25_V | (InvAb)psmApsmUmAmG(L4) | 277 | (VPmU)psfUpsmCpsmAmAfCm | 278 |
| ar79 | mCmUmAfCfAfAmAmCmCmAmGm | | UfGfGmUmUmUmGfUmAfGmAmC | |
| | UmUmGmApsmAps(InvAb) | | mUpsmApsmU | |
| M25_V | (InvAb)psmApsmUmAmGmUmC | 279 | (VPmU)psfUpsmCpsmAmAfCm | 280 |
| ar68 | (L4)mAfCfAfAmAmCmCmAmGm | | UfGfGmUmUmUmGfUmAfGmAmC | |
| | UmUmGmApsmAps(InvAb) | | mUpsmApsmU | |
| M25_V | (InvAb)psmApsmUmAmGmUmC | 281 | (VPmU)psfUpsmCpsmAmAfCm | 282 |
| ar80 | mUmAfCfAfAmAmCmCmAmG | | UfGfGmUmUmUmGfUmAfGmAmC | |
| | (L4)mUmGmApsmAps(InvAb) | | mUpsmApsmU | |
| M25_V | (InvAb)psmApsmUmAmGmUmC | 283 | (VPmU)psfUpsmCpsmAmAfCm | 284 |
| ar81 | mUmAfCfAfAmAmCmCmAmGmU | | UfGfGmUmUmUmGfUmAfGmAmC | |
| | (L4)mGmApsmAps(InvAb) | | mUpsmApsmU | |
| M25_V | (InvAb)psmAps(L13)mAmGm | 285 | (VPmU)psfUpsmCpsmAmAfCm | 286 |
| ar82 | UmCmUmAfCfAfAmAmCmCmAmG | | UfGfGmUmUmUmGfUmAfGmAmC | |
| | mUmUmGmApsmAps(InvAb) | | mUpsmApsmU | |
| M25_V | (InvAb)psmApsmUmAmG | 287 | (VPmU)psfUpsmCpsmAmAfCm | 288 |
| ar83 | (L13)mCmUmAfCfAfAmAmCmC | | UfGfGmUmUmUmGfUmAfGmAmC | |
| | mAmGmUmUmGmApsmAps | | mUpsmApsmU | |
| | (InvAb) | | | |
| M25_V | (InvAb)psmApsmUmAmGmUmC | 289 | (VPmU)psfUpsmCpsmAmAfCm | 290 |
| ar84 | (L13)mAfCfAfAmAmCmCmAmG | | UfGfGmUmUmUmGfUmAfGmAmC | |
| | mUmUmGmApsmAps(InvAb) | | mUpsmApsmU | |
| M25_V | (InvAb)psmApsmUmAmGmUmC | 291 | (VPmU)psfUpsmCpsmAmAfCm | 292 |
| ar85 | mUmAfCfAfAmAmCmCmAmG | | UfGfGmUmUmUmGfUmAfGmAmC | |
| | (L13)mUmGmApsmAps | | mUpsmApsmU | |
| | (InvAb) | | | |
| M25_V | (InvAb)psmApsmUmAmGmUmC | 293 | (VPmU)psfUpsmCpsmAmAfCm | 294 |
| ar86 | mUmAfCfAfAmAmCmCmAmGmU | | UfGfGmUmUmUmGfUmAfGmAmC | |
| | (L13)mGmApsmAps(InvAb) | | mUpsmApsmU | |
| M28_V | mApsmApsfGmUmGmUfGfGfCm | 317 | (VPmU)psfGpsmCmCmUfAmAm | 318 |
| ar1 | UmCfAmUmUmAmGfGmCmA | | UmGmAmGmCmCfAmCfAmCmUmU | |
| | (Chol4) | | psmGpsmG | |
| M28_V | mApsmApsfGmUmG(L4)fGfGf | 319 | (VPmU)psfGpsmCmCmUfAmAm | 320 |
| ar39 | CmUmCfAmUmUmAmGfGpsmCps | | UmGmAmGmCmCfAmCfAmCmUmU | |
| | mA | | psmGpsmG | |
| M28_V | mApsmApsfGmUmG(L7)fGfGf | 321 | (VPmU)psfGpsmCmCmUfAmAm | 322 |
| ar40 | CmUmCfAmUmUmAmGfGpsmCps | | UmGmAmGmCmCfAmCfAmCmUmU | |
| | mA | | psmGpsmG | |
| M28_V | mApsmApsfGmUmG(L5)fGfGf | 323 | (VPmU)psfGpsmCmCmUfAmAm | 324 |
| ar41 | CmUmCfAmUmUmAmGfGpsmCps | | UmGmAmGmCmCfAmCfAmCmUmU | |
| | mA | | psmGpsmG | |
| M28_V | mApsmApsfGmUmG(L6)fGfGf | 325 | (VPmU)psfGpsmCmCmUfAmAm | 326 |
| ar42 | CmUmCfAmUmUmAmGfGpsmCps | | UmGmAmGmCmCfAmCfAmCmUmU | |
| | mA | | psmGpsmG | |
| M28_V | mApsmApsfGmUmG(L13)fGfG | 327 | (VPmU)psfGpsmCmCmUfAmAm | 328 |
| ar50 | fCmUmCfAmUmUmAmGfGpsmCp | | UmGmAmGmCmCfAmCfAmCmUmU | |
| | smA | | psmGpsmG | |
| M96_V | (InvAb)psmGpsmAmAmGmA | 329 | (VPmU)psfUpsmAmCmGfUmCf | 330 |
| ar1 | (L4)fCmAfCfGfCmUmGmGmGm | | CfCmAmGmCmGfUmGfAmUmCmU | |
| | AmCmGmUpsmApsmA | | mUmCpsmCpsmA | |
| M96_V | (InvAb)psmGpsmAmAmGmAmU | 331 | (VPmU)psfUpsmAmCmGmUdCm | 332 |
| ar5 | (L22)mAfCfGfCmUmGmGmGmA | | CmCmAmGmCmGfUmGfAmUmCmU | |
| | mCmGmUmApsmAps(InvAb) | | psmUpsmC | |
| M165 | (InvAb)psmCpsmAmAmGmUmC | 333 | (VPmU)psfUpsmGmGmAmGdCm | 334 |
| Var5 | (L22)mAfAfGfAmUmCmGmGmC | | CmGmAmUmCmUfUmGfGmAmCmU | |
| | mUmCmCmApsmAps(InvAb) | | psmUpsmG | |
| M177 | (InvAb)psmUpsmGmCmAmA | 335 | (VPmU)psfUpsmGmGmUfUmUf | 336 |
| Var1 | (L21)fUmAfGfUfCmUmAmCmA | | GfUmAmGmAmCfUmAfUmUmUmG | |
| | mAmAmCmCpsmApsmA | | mCmApsmCpsmA | |
| M177 | (InvAb)psmUpsmGmCmAmAmA | 337 | (VPmU)psfUpsmGpsfGmUfUm | 338 |
| Var2 | (L4)mAfGfUfCmUmAmCmAmAm | | UfGmUfAmGfAmCfUmAfUmUfU | |
| | AmCmCmApsmAps(InvAb) | | mGmCpsmA | |
| M177 | (InvAb)psmUpsmGmCmAmAmA | 339 | (VPmU)psfUpsmGmGmUfUmUf | 340 |
| Var3 | (L4)mAfGfUfCmUmAmCmAmAm | | GfUmAmGmAmCfUmAfUmUmUmG | |
| | AmCmCmApsmAps(InvAb) | | psmCpsmA | |
| M177 | (InvAb)psmUpsmGmCmAmAmA | 341 | (VPmU)psfUpsmGmGmUmUdTm | 342 |
| Var5 | (L4)mAfGfUfCmUmAmCmAmAm | | GmUmAmGmAmCfUmAfUmUmUmG | |
| | AmCmCmApsmAps(InvAb) | | psmCpsmA | |
| M177 | (InvAb)psmUpsmGmCmAmAmA | 343 | (VPmU)psfUpsmGmGmUfUmUf | 344 |
| Var6 | (L4)mAfGfUfCmUmAmCmAmAm | | GfUmAmGmAmCfUdAfUmUmUmG | |
| | AmCmCmApsmAps(InvAb) | | psmCpsmA | |
| M25_V | (InvAb)psmApsmUmAmG(L4) | 345 | (VPmU)psfUpsmCmAmAfCmUf | 346 |
| ar93 | mCmUmAfCfAfAmAmCmCmAmGm | | GfGmUmUmUmGfUmAfGmAmCmU | |
| | UmUmGmApsmAps(InvAb) | | psmApsmU | |
| M25_V | (InvAb)psmApsmUmAmGmU | 347 | (VPmU)psfUpsmCmAmAfCmUf | 348 |
| ar94 | (L22)mUmAfCfAfAmAmCmCmA | | GfGmUmUmUmGfUmAfGmAmCmU | |
| | mGmUmUmGmApsmAps(InvAb) | | psmApsmU | |
| M25_V | (InvAb)psmApsmUmAmGmUmC | 349 | (VPmU)psfUpsmCmAmAfCmUf | 350 |
| ar95 | (L4)mAfCfAfAmAmCmCmAmGm | | GfGmUmUmUmGfUmAfGmAmCmU | |
| | UmUmGmApsmAps(InvAb) | | psmApsmU | |
| M25_V | (InvAb)psmApsmUmAmGmUmC | 351 | (VPmU)psfUpsmCmAmAfCmUf | 352 |
| ar98 | mUmAfCfAfAmA(L22)mCmAmG | | GfGmUmUmUmGfUmAfGmAmCmU | |
| | mUmUmGmApsmAps(InvAb) | | psmApsmU | |
| M25_V | (InvAb)psmApsmUmAmGmUmC | 353 | (VPmU)psfUpsmCmAmAfCmUf | 354 |
| ar99 | mUmAfCfAfAmAmC(L22)mAmG | | GfGmUmUmUmGfUmAfGmAmCmU | |
| | mUmUmGmApsmAps(InvAb) | | psmApsmU | |
| M25_V | (InvAb)psmApsmUmAmGmUmC | 355 | (VPmU)psfUpsmCmAmAfCmUf | 356 |
| ar101 | mUmAfCfAfAmAmCmCmA(L23) | | GfGmUmUmUmGfUmAfGmAmCmU | |
| | mUmUmGmApsmAps(InvAb) | | psmApsmU | |

Modification:
mN = 2′OMe;
fN = 2′F;
ps = phosphorothioate;
VP = vinyl phosphonate;
InvAb = inverse abasic

Stability of siRNA Conjugates in Mouse Brain Homogenates

Frozen brain tissues collected from C57BL/6J wild type mice were homogenized at 200 mg/mL tissue concentration in ice-cold homogenization buffer containing 100 mM Tris-HCl, pH6.0 buffer supplemented with 1 mM MgCl₂. Tissue homogenization was performed by adding 1 mL ice-cold homogenization buffer to a 2 mL tube (MP Biomedicals™) containing lysing Matrix D and one mouse brain hemisphere, followed by processing the tube in the FastPrep®-24 Instrument for 30 see at a speed of 5 m/s. Placing the samples on ice for 2 min and repeat the processing by FastPrep®-24 Instrument for 2 rounds. 100 μL of the homogenate was added to each well of a 96-well PCR plate, and 5 μL of 20 μM siRNA conjugate (final concentration 1 μM in the reaction) was added to each well, leave equal number of blank wells containing brain homogenates without adding siRNA conjugates. Place the PCR plate with samples in a Thermomixer and incubate at 37° C. for 24 h with shaking at 450 rpm. After the reaction, place the PCR plate on ice till samples are cool, add 5 μL of 20 uM siRNA conjugates to the blank wells containing brain homogenates (0-hr samples). Dilute the samples in 300 μL 10 mM EDTA (4-fold dilution) containing 1 μg/mL internal standard oligo, transfer the diluted samples to deep well 96 well plates. After well mix, transfer 50 μL samples into another deep well 96 well plates before starting the solid phase extraction procedure.

Stability of siRNA Conjugates in Human Liver Lysosomes and Rat Liver Tritosomes

Human Liver Lysosomes (Xenotech H0610.L) or rat liver tritosomes were thawed and diluted in 20 mM sodium citrate pH 5.0 buffer to 0.05 units/mL or 0.5 unites/mL acid phosphatase levels, respectively. Aliquot 50 μL of diluted human liver lysosomes or rat liver tritosomes into each well of 96 format PCR plate, followed by adding 5 μL of 10 μM siRNA conjugates (final concentration 1 μM in the reaction) into each well. Leave equal number of blank wells containing lysosomes or tritosomes without adding siRNA conjugates. Place the PCR plate with samples in a Thermomixer and incubate at 37° C. for 24 h with shaking at 450 rpm. After the reaction, place the PCR plate on ice till samples are cool, add 5 μL of 10 μM siRNA conjugates to blank wells containing lysosomes or tritosomes (0-hr samples). Dilute the samples in 300 μL 10 mM EDTA (4-fold dilution) containing 1 μg/mL internal standard oligo, transfer the diluted samples to deep well 96 well plates. After well mix, transfer 50 μL samples into another deep well 96 well plates before starting the solid phase extraction procedure.

### Sample Preparation Before Solid Phase Extraction

After stability assay, 50 μL samples were digested by adding 10 μL proteinase K solution (A4392,0010; PanReac Applichem) and 100 μL Tris-EDTA buffer (10 mM Tris, 1 mM EDTA) pH 8.0 (VWR; E112-500 ml) incubated at 60° C. with vortex at 1100 rpm for 30 minutes. After incubation, samples were diluted by adding 100 μL lysis buffer (Phenomenex, Cat AL0-8579) and 200 μL equilibration buffer (3.45 g Na₂HPO₄ in 500 mL water, adjusted to pH 5.5 with 1N NaOH)

### Solid Phase Extraction

Solid phase extraction was then performed using Clarity OTX solid phase extraction plates (Phenomenex, Cat. 8E-S103-EGA). The plates were conditioned by 1 mL methanol, followed by 1 mL equilibration buffer (3.45 g Na₂HPO₄ in 500 mL water, adjusted to pH 5.5 with 1N NaOH), then the samples were loaded onto the column. The column was then washed with 1 mL wash buffer (equilibration buffer pH 5.5, 50% v/v acetonitrile) for 3 times, then washed once with 0.5 mL water, and then washed once in 100 mM ammonium bicarbonate pH 10 supplemented with 0.56 g/LTCEP (tris(2-carboxyethyl) phosphine). Samples were eluted 3 times each with 0.5 mL elution buffer (200 mM ammonium bicarbonate pH 10 supplemented with TCEP (tris(2-carboxyethyl) phosphine), 50% v/v acetonitrile), and dried using nitrogen flow (TurboVap, 65 psi N2 at 70° C.).

### Analytical Method

After SPE, samples were reconstituted in dissolving buffer (300 μL 100 mM ammonium bicarbonate adjust pH 10, 10% v/v acetonitrile) and analysed using liquid chromatography combined with mass spectrometry detection AB Sciex TripleTOF 6600. Samples were injected (20 μL) and separated using an DNAPac™ RP Column 50 mm×2.1 mm, 4 m maintained at 75° C. Mobile phase A was 0.2% dimethyl butylamine, 0.5% hexafluoro isopropanol and 0.5% methanol and mobile phase B was acetonitrile/iso-propanol (95/5; v/v). Gradient of the solvent: time=0 min, 98% A+2% B; time=1.5 min, 98% A+2% B; time=4.5 min, 5% A+95% B; time=7.5% A+95% B; time=7.01 min, 98% A+2% B; time=10 min, 98% A+2% B. The ESI source was operated in negative ion mode, with full scan, using spray voltage=4500 V, Source temperature=350° C. Flow speed=0.25 mL per min. Analyst TF 1.8.1 software was used for data acquisition and Sciex OS 1.7.0 software was used for data processing.

### 7.5 Example 5: In Vivo Knockdown Efficiency Assessment of siRNA Conjugates in Mouse Models

The in vivo knockdown efficiencies of lipid-siRNA conjugates were assessed in mouse models with intracerebroventricular (ICV) injection following the procedure described below.

MAPT mRNA in seven brain regions (cortex, hippocampus, brainstem, cerebellum, striatum, midbrain, and cervical spinal cord) from hTAU KI mice at 7 days after a single ICV injection of M28_Var1, M28_Var39, M28_Var40, M28_Var41, M28_Var42, M28_Var50 at 15 nmol is assessed. The compounds are listed in Table 21 and results in Table 22. Data are presented as remaining MAPT mRNA (%). Mean±S.D., n=6 mice per treatment group.

### Mice and Intracerebroventricular Injection

Male and Female PS19 mice (C57BL6; Prnp-MAPT*P301S) or hTau KI (C57BL6; hMAPT: Knock-In) mice at age of 2-to-3-month are randomly assigned to different treatment groups. Mice are anesthetised with isoflurane (induction: 4-5%; maintenance: 1.8-2.5%). They are then stereotaxically injected using a motorized drill and microinjection robot (Neurostar, Germany, Sterodrive Sofeware v 2019) into the bilateral ventricles at coordinates: AP: −0.62 mm, ML: +/−1.05 mm; and DV: 2.2 mm. Each injection is performed in 5 μL volume over 5 min and following injection the needle is withdrawn in three steps (1.1 mm in 60 sec and kept there for 5 min; 2. another 0.5 mm in 30 sec, wait 5 min; 3. withdrawal out of the brain at very slow speed) to avoid compound reflux along the needle tract. After every step, the amount of backflow is checked. At selected post-injection days, animals are sacrificed and different brain regions including cortex, hippocampus, brainstem, cerebellum, striatum, midbrain, and cervical spinal cord are dissected and snap frozen and stored at −80° C. till further analysis.

RNA Extraction from Tissues

Tissues are collected in Lysing Matrix D (MP Biomedicals 6913-500) 2 mL Tubes containing 1.4 mm ceramic spheres and stored in −80° C. freezer till analysis. Place the tissues on ice under laminar flow and immediately add 750 μL Trizol (ThermoFisher 15596026/15596018) per tube. Disrupt and homogenize the tissue using the FastPrep-24™ 5G Grinder with 3 cycles of 30 sec at speed 5 m/sec. Between cycles, cool down the samples on ice for 2 minutes. After homogenization, shortly spin the tube and add 20% volume of Chloroform (150 μL Chloroform when 750 μL Trizol was used). Vortex for 15 sec and centrifuge at 14.000×g for 15 min at 4° C. Transfer the upper aqueous phase to a deep 96well plate and add 1 volume of 70% ethanol and mix well. The next steps are performed using RNeasy 96 kit (Qiagen) following manufacture's protocol. Place a RNeasy 96 plate on top of the Square-Well Block holder, apply the samples (Trizol/Chloroform extraction) into the wells of the RNeasy 96 plate and seal the plate with AirPore cover to prevent contamination. Centrifuge at 5600×g for 3 min at RT and discard the solution. Serial wash steps including 1 time of 800 μL RW1 buffer, 2 times of 800 μL RPE buffer that are applied to the RNeasy 96 plate, and wash buffers are removed by centrifugation at 5600×g for 3 min for each step. After the last wash, the RNeasy 96 plate is centrifuged at 5600×g for 3 min to remove the residual lipid. RNA is eluted using 60 μL RNase-free water by centrifugation at 5600×g for 3 min at RT. RNA concentration is measured by Nanodrop 8000 (ThermoFisher). The eluted RNA is stored at −80° C. until analysis.

### RT-qPCR

RNA is used as template for reverse transcription using reversed transcribed using High-Capacity cDNA Reverse Transcription Kits (Applied Biosystems) following manufacture's protocol. Briefly, a final reaction of 20 μL mixture is incubated at 25° C. for 10 min, followed by reverse transcription at 37° C. for 2 hours and enzyme inactivation at 85° C. for 5 min. For qPCR reaction, reverse transcribed cDNAs are diluted 10 times and mixed with 2× PowerUp™ SYBR™ Green Mater Mix (ThermoFisher A25743) and 500 nM qPCR primers to a final volume of 10 μL reaction. The qPCR runs are performed with a QuantStudio™ 12K instrument (Applied Biosystems™) using standard thermal cycling protocol. Multiple primers are used to detect MAPT mRNA, and reference primers targeting mouse housekeeping genes AP3D1 and PAK1IP1 are included for normalization of gene expression. The primer sequences are listed in Table 8. qPCR data are analysed using qBase+ software (Biogazelle).

TABLE 7
In vitro potency (IC50 values) of MAPT siRNA hits
in human neuroblastoma cells with transfection

| | MAPT_0T1 | | MAPT_B01 | | MAPT-2 | | |
|---|---|---|---|---|---|---|---|
| | (Exon 0-1) | | (Exon 11-12) | | (Exon 12-13) | | Target |
| | IC₅₀ | IC₅₀ 95% CI | IC₅₀ | IC₅₀ 95% CI | IC₅₀ | IC₅₀ 95% CI | gene |
| Name | pM | pM | pM | pM | pM | pM | region |
| M25 | 28 | 13.6 to 51.0 | 48.5 | 30.3 to 72.0 | 36.3 | 15.5 to 68.4 | Exon 11 |
| M28 | 9.5 | 4.0 to 18.9 | 18.0 | 7.5 to 35.1 | 7.8 | 10.2 to 28.9 | Exon 11 |
| M96 | 7.9 | 4.0 to 14.3 | 14.8 | 7.2 to 27.3 | 6.8 | 1.2 to 23.8 | Exon 1 |
| M165 | 4.5 | 1.8 to 9.6 | 4.2 | 1.5 to 9.9 | 2.2 | 0.45 to 7.2 | Exon 9 |
| M177 | 7.1 | 3.8 to 12.3 | 10.2 | 5.2 to 18.0 | 7.5 | 1.8 to 19.0 | Exon 11 |

TABLE 8
The sequences of qPCR primers

| | | Primer | Forward primer | SEQ ID | Reverse primer | SEQ ID |
|---|---|---|---|---|---|---|
| Target | Species | Name | (5′-3′) | NO | (5′-3′) | NO |
| GAPDH | Human | Hs_GAPDH | AAGGTGAAGGTCGGAGTCAAC | 295 | GGGGTCATTGATGGCAACAATA | 296 |
| ENOX2 | Human | Hs_ENOX2 | TCATTGTGGAAGTTTTCGAGCA | 297 | TGCGGTAACCAGACAGATACA | 298 |
| MAPT | Human | MAPT_B01 | CCTCCAAGTGTGGCTCATTA | 299 | CAATCTTCGACTGGACTCTG | 300 |
| MAPT | Human | MAPT_B02 | CAGTGGTCCGTACTCCA | 301 | TGGACTTGACATTCTTCAGG | 302 |
| MAPT | Human | MAPT_B04 | ATTGAAACCCACAAGCTGAC | 303 | GAGGAGACATTGCTGAGATG | 304 |
| MAPT | Human | MAPT_B06 | TCAGGTGAACTTTGAACCAG | 305 | CTTCCATCACTTCGAACTCC | 306 |
| MAPT | Human | MAPT_1 | CCAAGTGTGGCTCATTAGGCA | 307 | CCAATCTTCGACTGGACTCTGT | 308 |
| MAPT | Human | MAPT_2 | GAGTCCAGTCGAAGATTGGGT | 309 | GGCGAGTCTACCATGTCGATG | 310 |
| MAPT | Human | MAPT_0T1 | CACCACAGCCACCTTCTC | 311 | CTTCCATCACTTCGAACTCCT | 312 |
| AP3D1 | Mouse | Mm_AP3D1 | CAAGGGCAGTATCGACCGC | 313 | GATCTCGTCAATGCACTGGGA | 314 |
| PAK1IP1 | Mouse | Mm_PAK1IP1 | CTGTGGCTGATTTCACTCACC | 315 | CCCACTGATTAAGTGCTGGTTG | 316 |

TABLE 10
Knockdown efficiency of MAPT siRNA M25 chemical variants
(cholesterol-conjugates) in human iPSC-neurons

| | Remaining MAPT mRNA/control | | | | | |
|---|---|---|---|---|---|---|
| | 200 nM | | 40 nM | | 8 nM | |
| NAME | mean | SD | mean | SD | mean | SD |
| M25-Chol | 0.115 | 0.026 | 0.439 | 0.078 | 0.819 | 0.048 |
| M25_Var1-Chol | 0.046 | 0.003 | 0.219 | 0.036 | 0.573 | 0.012 |
| M25_Var2-Chol | 0.053 | 0.008 | 0.303 | 0.048 | 0.698 | 0.098 |
| M25_Var3-Chol | 0.104 | 0.019 | 0.530 | 0.106 | 0.777 | 0.059 |
| M25_Var4-Chol | 0.057 | 0.005 | 0.287 | 0.030 | 0.648 | 0.092 |
| M25_Var5-Chol | 0.737 | 0.020 | 0.994 | 0.097 | 0.926 | 0.077 |
| M25_Var6-Chol | 0.036 | 0.010 | 0.229 | 0.036 | 0.504 | 0.037 |
| M25_Var7-Chol | 0.043 | 0.001 | 0.268 | 0.048 | 0.551 | 0.016 |
| M25_Var8-Chol | 0.053 | 0.004 | 0.308 | 0.060 | 0.608 | 0.106 |
| M25_Var9-Chol | 0.228 | 0.027 | 0.789 | 0.052 | 0.916 | 0.005 |
| M25_Var10-Chol | 0.062 | 0.012 | 0.389 | 0.099 | 0.633 | 0.040 |
| M25_Var11-Chol | 0.044 | 0.002 | 0.323 | 0.057 | 0.646 | 0.012 |
| M25_Var12-Chol | 0.095 | 0.014 | 0.345 | 0.028 | 0.610 | 0.036 |
| M25_Var13-Chol | 0.858 | 0.108 | 1.083 | 0.116 | 0.971 | 0.086 |
| M25_Var14-Chol | 0.050 | 0.004 | 0.255 | 0.055 | 0.572 | 0.061 |
| M25_Var15-Chol | 0.076 | 0.014 | 0.375 | 0.075 | 0.583 | 0.077 |
| M25_Var16-Chol | 0.098 | 0.007 | 0.367 | 0.030 | 0.644 | 0.092 |
| M25_Var17-Chol | 0.067 | 0.005 | 0.296 | 0.006 | 0.675 | 0.051 |
| M25_Var18-Chol | 0.058 | 0.006 | 0.298 | 0.040 | 0.651 | 0.056 |
| M25_Var19-Chol | 0.053 | 0.002 | 0.305 | 0.071 | 0.642 | 0.081 |
| M25_Var20-Chol | 0.123 | 0.005 | 0.442 | 0.030 | 0.782 | 0.115 |
| M25_Var21-Chol | 0.097 | 0.007 | 0.396 | 0.024 | 0.696 | 0.103 |
| M25_Var22-Chol | 0.062 | 0.008 | 0.283 | 0.024 | 0.735 | 0.047 |
| M25_Var23-Chol | 0.070 | 0.012 | 0.412 | 0.039 | 0.747 | 0.059 |
| M25_Var24-Chol | 0.177 | 0.024 | 0.694 | 0.079 | 1.014 | 0.003 |
| M25_Var25-Chol | 0.103 | 0.005 | 0.410 | 0.054 | 0.761 | 0.084 |
| M25_Var26-Chol | 0.225 | 0.016 | 0.608 | 0.089 | 0.861 | 0.062 |
| M25_Var27-Chol | 0.054 | 0.004 | 0.205 | 0.014 | 0.586 | 0.010 |
| M25_Var28-Chol | 0.154 | 0.011 | 0.502 | 0.073 | 0.767 | 0.018 |
| M25_Var29-Chol | 0.139 | 0.003 | 0.470 | 0.074 | 0.824 | 0.058 |
| M25_Var30-Chol | 0.188 | 0.019 | 0.563 | 0.061 | 0.781 | 0.078 |
| M25_Var31-Chol | 0.143 | 0.017 | 0.425 | 0.025 | 0.791 | 0.080 |
| M25_Var32-Chol | 0.130 | 0.007 | 0.447 | 0.041 | 0.792 | 0.067 |
| M25_Var33-Chol | 0.263 | 0.005 | 0.686 | 0.116 | 0.917 | 0.091 |
| M25_Var34-Chol | 0.069 | 0.004 | 0.307 | 0.018 | 0.694 | 0.076 |
| M25_Var35-Chol | 0.958 | 0.164 | 0.877 | 0.090 | 1.033 | 0.090 |
| M25_Var36-Chol | 0.204 | 0.049 | 0.642 | 0.119 | 0.939 | 0.155 |
| M25_Var37-Chol | 0.178 | 0.012 | 0.522 | 0.081 | 0.873 | 0.049 |
| M25_Var38-Chol | 0.152 | 0.009 | 0.551 | 0.109 | 0.872 | 0.068 |
| M25_Var39-Chol | | | 0.665 | 0.056 | 0.801 | 0.101 |
| M25_Var40-Chol | 0.093 | 0.006 | 0.386 | 0.025 | 0.683 | 0.105 |
| M25_Var41-Chol | 0.047 | 0.006 | 0.191 | 0.017 | 0.515 | 0.087 |
| M25_Var42-Chol | 0.068 | 0.004 | 0.265 | 0.018 | 0.628 | 0.095 |
| M25_Var43-Chol | 0.111 | 0.011 | 0.471 | 0.069 | 0.766 | 0.040 |
| M25_Var44-Chol | 0.115 | 0.009 | 0.441 | 0.031 | 0.795 | 0.032 |
| M25_Var45-Chol | 0.267 | 0.016 | 0.670 | 0.075 | 0.780 | 0.053 |

TABLE 11
Knockdown efficiency of MAPT siRNA M28 chemical variants
(cholesterol-conjugates) in human iPSC-neurons

| | Remaining MAPT mRNA/control | | | | | |
|---|---|---|---|---|---|---|
| | 200 nM | | 40 nM | | 8 nM | |
| NAME | mean | SD | mean | SD | mean | SD |
| M28-Chol | 0.045 | 0.004 | 0.170 | 0.023 | 0.484 | 0.067 |
| M28_Var1-Chol | 0.036 | 0.006 | 0.121 | 0.021 | 0.354 | 0.058 |
| M28_Var2-Chol | 0.059 | 0.006 | 0.272 | 0.049 | 0.597 | 0.020 |
| M28_Var3-Chol | 0.066 | 0.008 | 0.302 | 0.079 | 0.629 | 0.081 |
| M28_Var4-Chol | 0.039 | 0.006 | 0.123 | 0.022 | 0.346 | 0.024 |
| M28_Var5-Chol | 0.069 | 0.014 | 0.275 | 0.042 | 0.572 | 0.023 |
| M28_Var6-Chol | 0.648 | 0.040 | 0.916 | 0.116 | 0.891 | 0.168 |
| M28_Var7-Chol | 0.034 | 0.003 | 0.092 | 0.018 | 0.292 | 0.045 |
| M28_Var8-Chol | 0.069 | 0.007 | 0.277 | 0.094 | 0.508 | 0.035 |
| M28_Var9-Chol | 0.064 | 0.006 | 0.201 | 0.022 | 0.508 | 0.044 |
| M28_Var10-Chol | 0.084 | 0.005 | 0.329 | 0.039 | 0.620 | 0.043 |
| M28_Var11-Chol | 0.044 | 0.008 | 0.168 | 0.035 | 0.398 | 0.034 |
| M28_Var12-Chol | 0.038 | 0.003 | 0.203 | 0.042 | 0.542 | 0.100 |
| M28_Var13-Chol | 0.047 | 0.007 | 0.216 | 0.029 | 0.604 | 0.089 |
| M28_Var14-Chol | 0.682 | 0.065 | 0.880 | 0.053 | 0.964 | 0.111 |
| M28_Var15-Chol | 0.056 | 0.004 | 0.213 | 0.013 | 0.532 | 0.017 |
| M28_Var16-Chol | 0.056 | 0.005 | 0.220 | 0.024 | 0.503 | 0.081 |
| M28_Var17-Chol | 0.064 | 0.006 | 0.254 | 0.019 | 0.581 | 0.119 |
| M28_Var18-Chol | 0.066 | 0.007 | 0.265 | 0.042 | 0.594 | 0.032 |
| M28_Var19-Chol | 0.053 | 0.002 | 0.221 | 0.023 | 0.520 | 0.027 |
| M28_Var20-Chol | 0.053 | 0.009 | 0.187 | 0.028 | 0.475 | 0.027 |
| M28_Var26-Chol | 0.038 | 0.005 | 0.158 | 0.034 | 0.384 | 0.026 |

TABLE 12
MAPT mRNA knockdown efficiency of MAPT siRNA
M28 lipid conjugates in human iPSC neurons

| | Remaining MAPT mRNA/control | | | | | |
|---|---|---|---|---|---|---|
| | 1000 nM | | 200 nM | | 40 nM | |
| NAME | mean | SD | mean | SD | mean | SD |
| M28-Chol | 0.024 | 0.003 | 0.038 | 0.005 | 0.121 | 0.018 |
| M28_Var1-Chol | 0.025 | 0.002 | 0.032 | 0.004 | 0.067 | 0.011 |
| M28_Var2-Chol | 0.026 | 0.002 | 0.053 | 0.006 | 0.146 | 0.009 |
| M28_Var29 | 0.042 | 0.004 | 0.127 | 0.016 | 0.568 | 0.015 |
| M28_Var30 | 0.030 | 0.001 | 0.065 | 0.009 | 0.300 | 0.061 |
| M28_Var33 | 0.109 | 0.008 | 0.429 | 0.015 | / | / |
| M28_Var34 | 0.037 | 0.001 | 0.164 | 0.046 | 0.568 | 0.024 |
| M28_Var35 | 0.029 | 0.003 | 0.042 | 0.007 | 0.124 | 0.019 |
| M28_Var36 | 0.034 | 0.004 | 0.080 | 0.006 | 0.255 | 0.003 |
| M28_Var37 | 0.322 | 0.025 | 0.645 | 0.051 | 0.884 | 0.049 |
| M28_Var38 | 0.347 | 0.041 | 0.648 | 0.016 | 0.913 | 0.085 |
| M28_Var39 | 0.305 | 0.053 | 0.562 | 0.059 | 0.916 | 0.135 |
| M28_Var40 | 0.353 | 0.022 | 0.651 | 0.040 | 0.787 | 0.025 |
| M28_Var41 | 0.416 | 0.053 | 0.751 | 0.062 | 0.887 | 0.076 |
| M28_Var42 | 0.428 | 0.057 | 0.683 | 0.111 | 0.850 | 0.052 |
| M28_Var43 | 0.038 | 0.004 | 0.168 | 0.053 | 0.681 | 0.144 |
| M28_Var44 | 0.188 | 0.011 | 0.605 | 0.047 | 0.884 | 0.124 |
| M28_Var45 | 0.403 | 0.020 | 0.814 | 0.166 | 0.922 | 0.075 |
| M28_Var46 | 0.286 | 0.018 | 0.723 | 0.029 | 0.882 | 0.057 |
| M28_Var47 | 0.390 | 0.022 | 0.831 | 0.087 | 1.002 | 0.056 |
| M28_Var48 | 0.450 | 0.026 | 0.791 | 0.050 | 0.927 | 0.087 |
| M28_Var49 | 0.027 | 0.003 | 0.072 | 0.016 | 0.340 | 0.018 |

TABLE 13
MAPT mRNA knockdown efficiency of MAPT siRNA
M25 lipid conjugates in human iPSC neurons

| | Remaining MAPT mRNA/control | | | | | |
|---|---|---|---|---|---|---|
| | 1000 nM | | 200 nM | | 40 nM | |
| NAME | mean | SD | mean | SD | mean | SD |
| M25_Var46 | 0.187 | 0.064 | 0.649 | 0.040 | 0.968 | 0.193 |
| M25_Var47 | 0.034 | 0.007 | 0.039 | 0.006 | 0.220 | 0.067 |
| M25_Var48 | 0.182 | 0.015 | 0.682 | 0.080 | 0.708 | 0.047 |
| M25_Var49 | 0.679 | 0.053 | 0.954 | 0.095 | 0.864 | 0.120 |
| M25_Var50 | 0.041 | 0.009 | 0.218 | 0.032 | 0.662 | 0.062 |
| M25_Var51 | 0.038 | 0.004 | 0.240 | 0.056 | 0.728 | 0.075 |
| M25_Var52 | 0.068 | 0.018 | 0.390 | 0.054 | 0.889 | 0.213 |
| M25_Var53 | 0.641 | 0.041 | 0.841 | 0.066 | 1.034 | 0.052 |
| M25_Var54 | 0.149 | 0.036 | 0.529 | 0.063 | 0.882 | 0.028 |
| M25_Var55 | 0.718 | 0.060 | 0.979 | 0.084 | 0.948 | 0.052 |
| M25_Var56 | 0.503 | 0.028 | 0.878 | 0.006 | 1.077 | 0.034 |
| M25_Var57 | 0.548 | 0.056 | 0.868 | 0.060 | 0.929 | 0.043 |
| M25_Var59 | 0.626 | 0.055 | 0.884 | 0.118 | 0.984 | 0.077 |
| M25_Var60 | 0.621 | 0.031 | 0.897 | 0.109 | 1.051 | 0.180 |
| M25_Var62 | 0.300 | 0.034 | 0.706 | 0.058 | 0.910 | 0.103 |
| M25_Var63 | 0.331 | 0.040 | 0.701 | 0.031 | 1.001 | 0.065 |
| M25_Var64 | 0.202 | 0.030 | 0.718 | 0.086 | 0.882 | 0.106 |
| M25_Var66 | 0.381 | 0.032 | 0.755 | 0.058 | 0.925 | 0.066 |
| M25_Var68 | 0.590 | 0.103 | 0.778 | 0.087 | 0.935 | 0.126 |

TABLE 14
MAPT mRNA knockdown efficiency of MAPT siRNA M25 lipid
conjugates (lipid walk) in human iPSC neurons

| | Remaining MAPT mRNA/control | | | | | |
|---|---|---|---|---|---|---|
| | 1000 nM | | 200 nM | | 40 nM | |
| NAME | mean | SD | mean | SD | mean | SD |
| M25_Var73 | 0.566 | 0.051 | 0.801 | 0.061 | 0.928 | 0.046 |
| M25_Var74 | 0.706 | 0.017 | 0.774 | 0.085 | 1.003 | 0.061 |
| M25_Var75 | 0.532 | 0.080 | 0.757 | 0.025 | 1.021 | 0.076 |
| M25_Var76 | 0.536 | 0.056 | 0.819 | 0.152 | 1.037 | 0.160 |
| M25_Var77 | 0.572 | 0.073 | 0.759 | 0.090 | 0.841 | 0.160 |
| M25_Var78 | 0.608 | 0.014 | 0.919 | 0.159 | 0.944 | 0.082 |
| M25_Var79 | 0.556 | 0.055 | 0.763 | 0.096 | 0.826 | 0.063 |
| M25_Var68 | 0.553 | 0.044 | 0.870 | 0.070 | 0.986 | 0.050 |
| M25_Var80 | 0.502 | 0.116 | 0.717 | 0.059 | 0.986 | 0.068 |
| M25_Var81 | 0.551 | 0.059 | 0.749 | 0.145 | 0.822 | 0.091 |
| M25_Var82 | 0.572 | 0.060 | 0.841 | 0.060 | 0.911 | 0.150 |
| M25_Var83 | 0.662 | 0.030 | 0.962 | 0.064 | 1.090 | 0.153 |
| M25_Var84 | 0.507 | 0.046 | 0.721 | 0.127 | 0.830 | 0.081 |
| M25_Var85 | 0.432 | 0.026 | 0.831 | 0.087 | 0.891 | 0.173 |
| M25_Var86 | 0.577 | 0.059 | 0.777 | 0.147 | 1.053 | 0.071 |

TABLE 15
LC-MS measurement of MAPT siRNA stability
in mouse brain homogenates

| | % Stability | | | | | |
|---|---|---|---|---|---|---|
| | Antisense strand | | | Sense strand | | |
| NAME | Mean | SD | N | Mean | SD | N |
| M25-Chol | 61.3 | 8.3 | 9 | 65.9 | 11.9 | 9 |
| M25_Var1-Chol | 58.9 | 7.6 | 6 | 88.1 | 4.5 | 6 |
| M25_Var2-Chol | 39.9 | 9.7 | 6 | 43.7 | 19.3 | 6 |
| M25_Var3-Chol | 54.7 | 13.7 | 6 | 49.1 | 42.1 | 6 |
| M25_Var4-Chol | 73.1 | 10.9 | 6 | 71.7 | 36.6 | 6 |
| M25_Var5-Chol | 73.6 | 7.3 | 3 | 57.1 | 8.1 | 3 |
| M25_Var6-Chol | 55.2 | 7.0 | 6 | 41.9 | 10.1 | 6 |
| M25_Var7-Chol | 73.1 | 12.6 | 6 | 39.4 | 7.3 | 6 |
| M25_Var8-Chol | 69.1 | 8.4 | 6 | 67.1 | 20.7 | 6 |
| M25_Var9-Chol | 68.9 | 10.6 | 3 | 28.9 | 8.1 | 3 |
| M25_Var10-Chol | 82.4 | 11.9 | 3 | 64.5 | 9.6 | 3 |
| M25_Var11-Chol | 47.3 | 2.9 | 6 | 64.1 | 14.7 | 6 |
| M25_Var12-Chol | 68.5 | 11.5 | 6 | 76.7 | 13.7 | 6 |
| M25_Var13-Chol | 70.9 | 1.8 | 3 | 101.1 | 2.9 | 3 |
| M25_Var14-Chol | 71.8 | 12.2 | 6 | 65.0 | 18.6 | 6 |
| M25_Var15-Chol | 88.8 | 7.7 | 9 | 64.2 | 12.7 | 9 |
| M25_Var16-Chol | 68.6 | 3.7 | 6 | 60.4 | 5.6 | 6 |
| M25_Var17-Chol | 79.1 | 13.1 | 6 | 91.0 | 11.2 | 6 |
| M25_Var18-Chol | 75.9 | 9.2 | 3 | 84.3 | 4.4 | 3 |
| M25_Var19-Chol | 69.9 | 6.9 | 3 | 88.9 | 7.7 | 3 |
| M25_Var20-Chol | 87.2 | 5.5 | 3 | 51.9 | 3.7 | 3 |
| M25_Var21-Chol | 53.9 | 7.2 | 3 | 70.6 | 1.3 | 3 |
| M25_Var22-Chol | 78.8 | 7.4 | 3 | 48.3 | 4.9 | 3 |
| M25_Var23-Chol | 86.8 | 5.9 | 3 | 59.0 | 7.8 | 3 |
| M25_Var24-Chol | 41.2 | 4.0 | 3 | 75.9 | 2.2 | 3 |
| M25_Var25-Chol | 64.3 | 12.1 | 3 | 60.6 | 13.7 | 3 |
| M25_Var26-Chol | 40.9 | 8.7 | 3 | 62.3 | 19.2 | 3 |
| M25_Var27-Chol | 57.4 | 5.3 | 6 | 54.4 | 9.9 | 6 |
| M25_Var49 | 57.3 | 2.6 | 3 | 55.5 | 3.2 | 3 |
| M25_Var84 | 78.6 | 5.1 | 3 | 98.7 | 4.7 | 3 |
| M25_Var68 | 78.0 | 4.4 | 3 | 86.5 | 1.0 | 3 |
| MSC-2-V | 81.9 | 11.1 | 24 | 63.0 | 13.0 | 24 |

TABLE 16
LC-MS measurement of MAPT siRNA stability
in human liver tritosome

| | % Stability | | | | | |
|---|---|---|---|---|---|---|
| | Antisense strand | | | Sense strand | | |
| NAME | Mean | SD | N | Mean | SD | N |
| M25-Chol | 77.1 | 11.1 | 3 | 73.2 | 2.8 | 3 |
| M25_Var1-Chol | 66.5 | 4.0 | 3 | 38.1 | 0.9 | 3 |
| M25_Var6-Chol | 81.4 | 15.0 | 3 | 81.0 | 16.0 | 3 |
| M25_Var7-Chol | 80.9 | 1.6 | 3 | 75.6 | 8.1 | 3 |
| M25_Var12-Chol | 80.7 | 5.3 | 3 | 82.9 | 7.2 | 3 |
| M25_Var15-Chol | 96.1 | 3.7 | 3 | 88.9 | 9.5 | 3 |
| M25_Var16-Chol | 90.5 | 11.7 | 3 | 88.5 | 8.4 | 3 |
| M25_Var66 | 97.4 | 13.1 | 3 | 90.8 | 7.4 | 3 |
| M25_Var68 | 89.6 | 2.1 | 3 | 87.3 | 2.5 | 3 |
| MSC-2-V | 90.0 | 4.8 | 6 | 3.0 | 1.0 | 6 |

TABLE 17
LC-MS measurement of MAPT siRNA
stability in rat liver tritosome

| | % Stability | | | | | |
|---|---|---|---|---|---|---|
| | Antisense strand | | | Sense strand | | |
| NAME | Mean | SD | N | Mean | SD | N |
| M25-Chol | 78.8 | 8.3 | 3 | 81.1 | 3.8 | 3 |
| M25_Var1-Chol | 95.6 | 4.6 | 3 | 91.6 | 4.1 | 3 |
| M25_Var6-Chol | 84.2 | 13.0 | 3 | 83.3 | 4.2 | 3 |
| M25_Var7-Chol | 103.4 | 10.9 | 3 | 93.8 | 4.3 | 3 |
| M25_Var12-Chol | 90.8 | 6.0 | 3 | 97.8 | 1.9 | 3 |
| M25_Var15-Chol | 88.1 | 11.9 | 3 | 90.6 | 12.2 | 3 |
| M25_Var16-Chol | 103.4 | 6.9 | 3 | 96.8 | 3.1 | 3 |
| M25_Var66 | 91.4 | 13.5 | 3 | 90.7 | 11.1 | 3 |
| M25_Var68 | 90.9 | 15.8 | 3 | 94.8 | 7.4 | 3 |
| MSC-2-V | 82.0 | 5.7 | 6 | 17.3 | 13.5 | 6 |

TABLE 21
The modified sense and antisense strand sequences of
lipid conjugated MAPT siRNA

| | Modified sequences (5′ - 3′) | | | |
|---|---|---|---|---|
| | | SEQ ID | | SEQ ID |
| NAME | Sense strand | NO | Antisense strand | NO |
| M28_V | mApsmApsfGmUmGmUfGfGfCm | 317 | (VPmU)psfGpsmCmCmUfAmAm | 318 |
| ar1 | UmCfAmUmUmAmGfGmCmA | | UmGmAmGmCmCfAmCfAmCmUmU | |
| | (Chol4) | | psmGpsmG | |
| M28_V | mApsmApsfGmUmG(L4)fGfGf | 319 | (VPmU)psfGpsmCmCmUfAmAm | 320 |
| ar39 | CmUmCfAmUmUmAmGfGpsmCps | | UmGmAmGmCmCfAmCfAmCmUmU | |
| | mA | | psmGpsmG | |
| M28_V | mApsmApsfGmUmG(L7)fGfGf | 321 | (VPmU)psfGpsmCmCmUfAmAm | 322 |
| ar40 | CmUmCfAmUmUmAmGfGpsmCps | | UmGmAmGmCmCfAmCfAmCmUmU | |
| | mA | | psmGpsmG | |
| M28_V | mApsmApsfGmUmG(L5)fGfGf | 323 | (VPmU)psfGpsmCmCmUfAmAm | 324 |
| ar41 | CmUmCfAmUmUmAmGfGpsmCps | | UmGmAmGmCmCfAmCfAmCmUmU | |
| | mA | | psmGpsmG | |
| M28_V | mApsmApsfGmUmG(L6)fGfGf | 325 | (VPmU)psfGpsmCmCmUfAmAm | 326 |
| ar42 | CmUmCfAmUmUmAmGfGpsmCps | | UmGmAmGmCmCfAmCfAmCmUmU | |
| | mA | | psmGpsmG | |
| M28_V | mApsmApsfGmUmG(L13)fGfG | 327 | (VPmU)psfGpsmCmCmUfAmAm | 328 |
| ar50 | fCmUmCfAmUmUmAmGfGpsmCp | | UmGmAmGmCmCfAmCfAmCmUmU | |
| | smA | | psmGpsmG | |

(Modification: mN = 2′OMe; fN = 2′F; ps = phosphorothioate; VP = vinyl phosphonate; InvAb = inverse abasic)

TABLE 22
In vivo knockdown efficiency of lipid-M28Var conjugates in hTAU KI mice
at 7 days after ICV injection (15 nmol siRNA conjugate). Data are presented
as remaining MAPT mRNA (%) levels; n = 6 mice per treatment group.

| | Remaining MAPT mRNA (%) | | | | | | | | | | | | | |
|---|---|---|---|---|---|---|---|---|---|---|---|---|---|---|
| | Cortex | | Hippocampus | | Cerebellum | | Spinal cord | | Striatum | | Midbrain | | Brainstem | |
| NAME | mean | SD | mean | SD | mean | SD | mean | SD | mean | SD | mean | SD | mean | SD |
| Vehicle | 100.0 | 4.6 | 100.0 | 10.6 | 100.0 | 6.0 | 100.0 | 10.6 | 100.0 | 5.7 | 100.0 | 7.6 | 100.0 | 6.7 |
| M28_Var1-Chol | 58.6 | 4.8 | 35.5 | 4.8 | 53.3 | 6.3 | 30.8 | 1.8 | 60.6 | 9.4 | 42.0 | 3.2 | 41.6 | 5.2 |
| M28_Var39 | 41.0 | 10.8 | 28.8 | 5.9 | 38.2 | 6.7 | 21.4 | 2.7 | 39.6 | 4.1 | 31.2 | 3.9 | 29.7 | 2.1 |
| M28_Var41 | 36.7 | 7.0 | 25.4 | 3.6 | 36.5 | 7.2 | 19.9 | 0.8 | 36.4 | 3.1 | 27.4 | 2.2 | 26.1 | 2.4 |
| M28_Var40 | 33.2 | 8.1 | 23.5 | 6.1 | 37.1 | 7.4 | 21.3 | 2.1 | 36.0 | 7.8 | 26.3 | 3.2 | 29.5 | 2.7 |
| M28_Var42 | 40.2 | 5.3 | 26.3 | 2.3 | 35.6 | 1.3 | 22.8 | 4.4 | 40.4 | 5.6 | 28.4 | 3.1 | 26.2 | 2.2 |
| M28_Var50 | 34.9 | 9.3 | 26.8 | 6.4 | 38.7 | 14.2 | 21.0 | 1.3 | 38.2 | 9.9 | 29.9 | 5.9 | 27.8 | 5.4 |

TABLE 23
The modified sense and antisense strand sequences of
lipid conjugated MAPT siRNA

| | Modified sequences (5′ - 3′) | | | |
|---|---|---|---|---|
| | | SEQ ID | | SEQ ID |
| NAME | Sense strand | NO. | Antisense strand | NO. |
| M96_V | (InvAb)psmGpsmAmAmGmA(L4)f | 329 | (VPmU)psfUpsmAmCmGfUmCfCf | 330 |
| ar1 | CmAfCfGfCmUmGmGmGmAmCmGmUp | | CmAmGmCmGfUmGfAmUmCmUmUmC | |
| | smApsmA | | psmCpsmA | |
| M96_V | (InvAb)psmGpsmAmAmGmAmU | 331 | (VPmU)psfUpsmAmCmGmUdCmCm | 332 |
| ar5 | (L22)mAfCfGfCmUmGmGmGmAmCm | | CmAmGmCmGfUmGfAmUmCmUpsmU | |
| | GmUmApsmAps(InvAb) | | psmC | |
| M165 | (InvAb)psmCpsmAmAmGmUmC | 333 | (VPmU)psfUpsmGmGmAmGdCmCm | 334 |
| Var5 | (L22)mAfAfGfAmUmCmGmGmCmUm | | GmAmUmCmUfUmGfGmAmCmUpsmU | |
| | CmCmApsmAps(InvAb) | | psmG | |
| M177 | (InvAb)psmUpsmGmCmAmA(L21) | 335 | (VPmU)psfUpsmGmGmUfUmUfGf | 336 |
| Var1 | fUmAfGfUfCmUmAmCmAmAmAmCm | | UmAmGmAmCfUmAfUmUmUmGmCmA | |
| | CpsmApsmA | | psmCpsmA | |
| M177 | (InvAb)psmUpsmGmCmAmAmA | 337 | (VPmU)psfUpsmGpsfGmUfUmUf | 338 |
| Var2 | (L4)mAfGfUfCmUmAmCmAmAmAmC | | GmUfAmGfAmCfUmAfUmUfUmGmC | |
| | mCmApsmAps(InvAb) | | psmA | |
| M177 | (InvAb)psmUpsmGmCmAmAmA | 339 | (VPmU)psfUpsmGmGmUfUmUfGf | 340 |
| Var3 | (L4)mAfGfUfCmUmAmCmAmAmAmC | | UmAmGmAmCfUmAfUmUmUmGpsmC | |
| | mCmApsmAps(InvAb) | | psmA | |
| M177 | (InvAb)psmUpsmGmCmAmAmA | 341 | (VPmU)psfUpsmGmGmUmUdTmGm | 342 |
| Var5 | (L4)mAfGfUfCmUmAmCmAmAmAmC | | UmAmGmAmCfUmAfUmUmUmGpsmC | |
| | mCmApsmAps(InvAb) | | psmA | |
| M177 | (InvAb)psmUpsmGmCmAmAmA | 343 | (VPmU)psfUpsmGmGmUfUmUfGf | 344 |
| Var6 | (L4)mAfGfUfCmUmAmCmAmAmAmC | | UmAmGmAmCfUdAfUmUmUmGpsmC | |
| | mCmApsmAps(InvAb) | | psmA | |
| M25_V | (InvAb)psmApsmUmAmG(L4)mCm | 345 | (VPmU)psfUpsmCmAmAfCmUfGf | 346 |
| ar93 | UmAfCfAfAmAmCmCmAmGmUmUmGm | | GmUmUmUmGfUmAfGmAmCmUpsmA | |
| | ApsmAps(InvAb) | | psmU | |
| M25_V | (InvAb)psmApsmUmAmGmU(L22) | 347 | (VPmU)psfUpsmCmAmAfCmUfGf | 348 |
| ar94 | mUmAfCfAfAmAmCmCmAmGmUmUmG | | GmUmUmUmGfUmAfGmAmCmUpsmA | |
| | mApsmAps(InvAb) | | psmU | |
| M25_V | (InvAb)psmApsmUmAmGmUmC | 349 | (VPmU)psfUpsmCmAmAfCmUfGf | 350 |
| ar95 | (L4)mAfCfAfAmAmCmCmAmGmUmU | | GmUmUmUmGfUmAfGmAmCmUpsmA | |
| | mGmApsmAps(InvAb) | | psmU | |
| M25_V | (InvAb)psmApsmUmAmGmUmCmUm | 351 | (VPmU)psfUpsmCmAmAfCmUfGf | 352 |
| ar98 | AfCfAfAmA(L22)mCmAmGmUmUmG | | GmUmUmUmGfUmAfGmAmCmUpsmA | |
| | mApsmAps(InvAb) | | psmU | |
| M25_V | (InvAb)psmApsmUmAmGmUmCmUm | 353 | (VPmU)psfUpsmCmAmAfCmUfGf | 354 |
| ar99 | AfCfAfAmAmC(L22)mAmGmUmUmG | | GmUmUmUmGfUmAfGmAmCmUpsmA | |
| | mApsmAps(InvAb) | | psmU | |
| M25_V | (InvAb)psmApsmUmAmGmUmCmUm | 355 | (VPmU)psfUpsmCmAmAfCmUfGf | 356 |
| ar101 | AfCfAfAmAmCmCmA(L23)mUmUmG | | GmUmUmUmGfUmAfGmAmCmUpsmA | |
| | mApsmAps(InvAb) | | psmU | |

(Modification: mN = 2′OMe; fN = 2′F; ps = phosphorothioate; VP = vinyl phosphonate; InvAb = inverse abasic)

TABLE 24
LC-MS measurement of MAPT siRNA stability
in mouse brain homogenate.

% Stability

| | Antisense strand | | | Sense strand | | |
|---|---|---|---|---|---|---|
| NAME | Mean | SD | N | Mean | SD | N |
| M96_Var1 | 53.9 | 6.2 | 6 | 73.2 | 10.2 | 6 |
| M96_Var5 | 89.2 | 17.7 | 6 | 90.0 | 20.5 | 6 |
| M165_Var5 | 87.6 | 13.1 | 6 | 91.6 | 6.9 | 6 |
| M177_Var1 | 74.2 | 4.3 | 6 | 76.3 | 13.7 | 6 |
| M177_Var2 | 89.5 | 7.4 | 6 | 75.4 | 12.6 | 6 |
| M177_Var3 | 88.7 | 4.5 | 6 | 85.7 | 10.9 | 6 |
| M177_Var5 | 90.4 | 6.6 | 6 | 82.7 | 5.4 | 6 |
| MSC-2-V | 74.1 | 5.0 | 6 | 46.8 | 8.3 | 6 |

TABLE 25
LC-MS measurement of MAPT siRNA stability
in human liver tritosome.

% Stability

| | Antisense strand | | | Sense strand | | |
|---|---|---|---|---|---|---|
| NAME | Mean | SD | N | Mean | SD | N |
| M96_Var1 | 100.7 | 7.9 | 3 | 99.1 | 8.0 | 3 |
| M96_Var5 | 95.8 | 5.7 | 3 | 90.1 | 3.0 | 3 |
| M165_Var5 | 94.4 | 7.5 | 3 | 84.3 | 7.0 | 3 |
| M177_Var1 | 103.3 | 16.2 | 3 | 95.8 | 25.9 | 3 |
| M177_Var2 | 101.7 | 5.9 | 3 | 100.1 | 9.9 | 3 |
| M177_Var3 | 104.7 | 4.6 | 3 | 94.8 | 4.2 | 3 |
| M177_Var5 | 87.7 | 7.2 | 3 | 82.7 | 8.7 | 3 |
| MSC-2-V | 94.6 | 4.4 | 3 | 0.7 | 0.2 | 3 |

TABLE 26
LC-MS measurement of MAPT siRNA
stability in rat liver tritosome.

% Stability

| | Antisense strand | | | Sense strand | | |
|---|---|---|---|---|---|---|
| Name | Mean | SD | N | Mean | SD | N |
| M96_Var1 | 116.3 | 7.8 | 3 | 116.5 | 8.9 | 3 |
| M96_Var5 | 105.9 | 10.0 | 3 | 100.2 | 5.4 | 3 |
| M165_Var5 | 109.0 | 9.5 | 3 | 104.2 | 12.8 | 3 |
| M177_Var1 | 111.3 | 7.6 | 3 | 103.8 | 8.4 | 3 |
| M177_Var2 | 110.3 | 3.1 | 3 | 111.0 | 8.0 | 3 |
| M177_Var3 | 99.4 | 2.0 | 3 | 112.1 | 25.0 | 3 |
| M177_Var5 | 118.3 | 7.8 | 3 | 116.0 | 11.3 | 3 |
| MSC-2-V | 119.3 | 5.0 | 3 | 1.2 | 0.4 | 3 |

### 7.6 Example 6: In Vivo Duration of Effects of siRNA Conjugates in a Human Tau Mouse Model

The in vivo duration of effects of lipid-siRNA conjugates were assessed in a human Tau mouse model with intracerebroventricular (ICV) injection following the procedure described below.

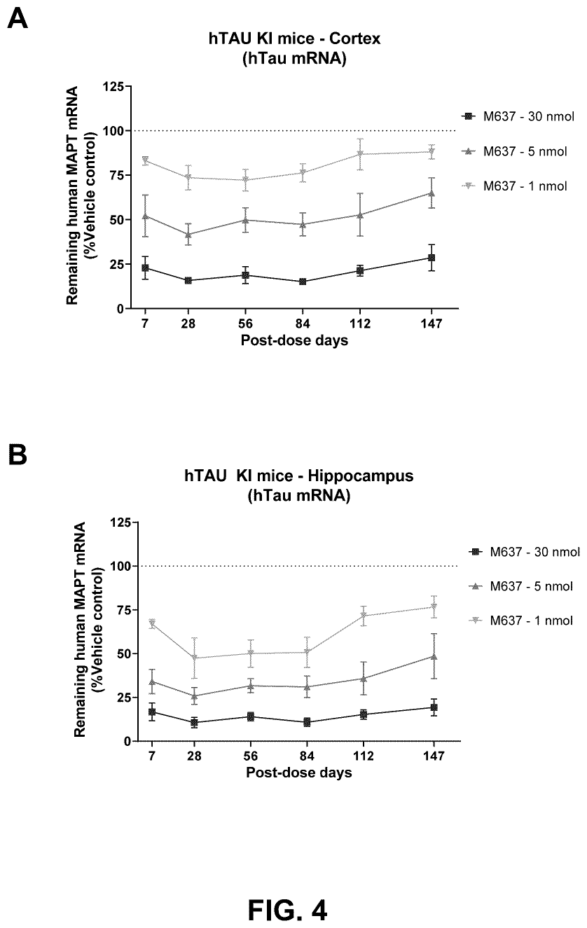

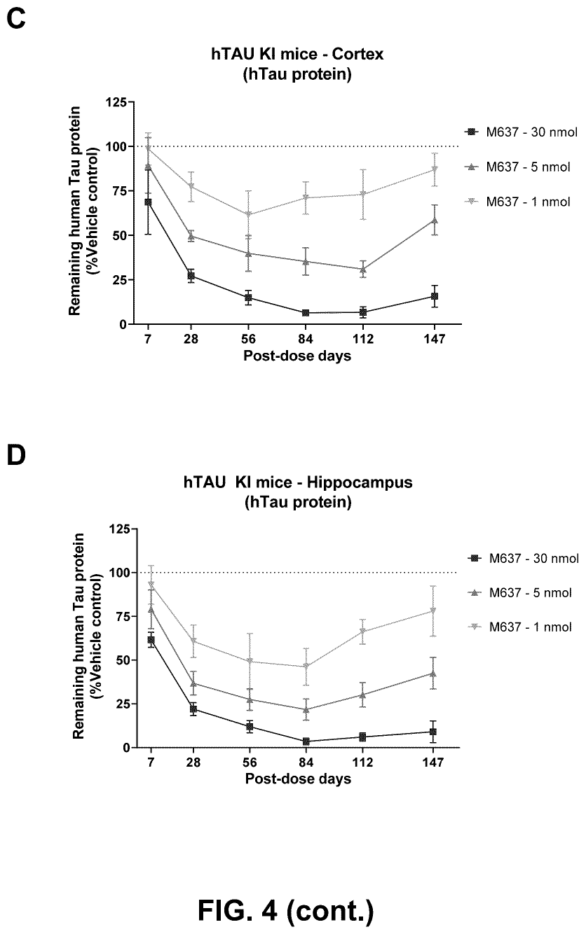

FIG. 4A-4D and Tables 27-30 summarize the levels of human MAPT mRNA and Tau protein in cortex and hippocampus from hTAU KI mice at 7-, 28-, 56-, 84-, 112-, and 147-days after a single ICV injection of M637 (i.e., M177_Var1, which is described in Table 5) at 30 nmol, 5 nmol and 1 nmol. The data in Tables 27-30 are presented as remaining MAPT mRNA (% vehicle control) or remaining Tau protein (% vehicle control). Mean±S.D., n=5 mice per treatment group. Sustained effects of M637 on target knockdown are observed from 7- to 147-days in hTAU KI mice.

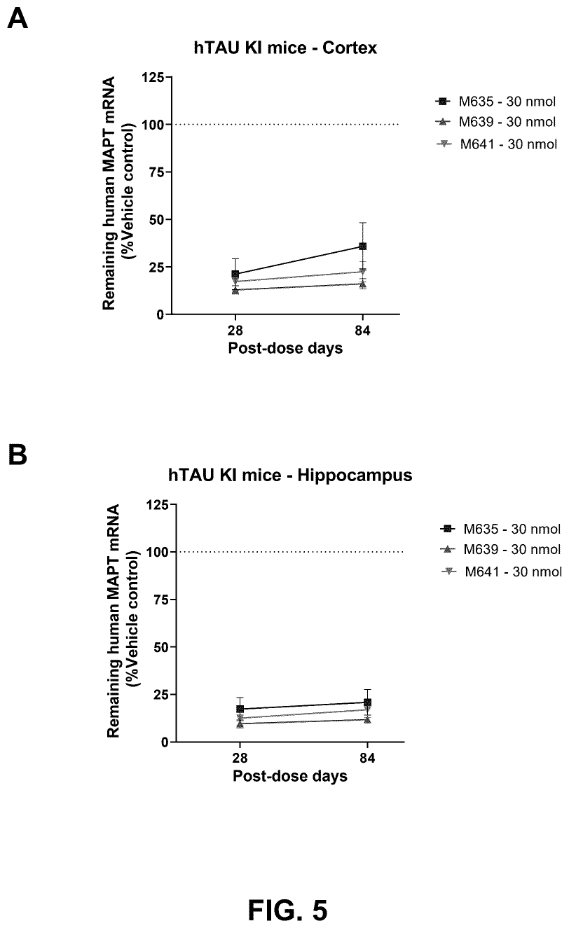

FIGS. 5A and 5B and Tables 31 and 32 summarize the levels of human MAPT mRNA in cortex and hippocampus from hTAU KI mice at 28-, 84-days after a single ICV injection of M635, M639, and M641 (M165_Var5, M177_Var3, and M177_Var5, respectively, which are described in Table 5) at 30 nmol. The data in Tables 31 and 32 are presented as remaining MAPT mRNA (% vehicle control). Mean±S.D., n=4-5 mice per treatment group. Sustained effects of M635, M639, and M641 on target knockdown are observed from 28- to 84-days in hTAU KI mice.

### Mice and Intracerebroventricular Injection

Male and Female hTau KI (C57BL6; hMAPT: Knock-In) mice at age of 2-to-3-months were randomly assigned to different treatment groups. Mice were anesthetised with isoflurane (induction: 4-5%; maintenance: 1.8-2.5%). They were then stereotaxically injected using a motorized drill and microinjection robot (Neurostar, Germany, Sterodrive Sofeware v 2019) into the bilateral ventricles at coordinates: AP: −0.62 mm, ML: +/−1.05 mm; and DV: 2.2 mm. Each injection was performed in 5 μL volume over 5 min and, following injection, the needle was withdrawn in three steps—(1) 1 mm in 60 sec and kept there for 5 min; (2) another 0.5 mm in 30 sec, wait 5 min; (3) withdrawal out of the brain at very slow speed—to avoid compound reflux along the needle tract. After every step the amount of backflow was checked. At selected post-injection days, animals were sacrificed and different brain regions including cortex, hippocampus, brainstem, cerebellum, striatum, midbrain, and cervical spinal cord were dissected and snap frozen and stored at −80° C. till further analysis.

RNA Extraction from Tissues

Tissues were collected in Lysing Matrix D (MP Biomedicals 6913-500) 2 mL tubes containing 1.4 mm ceramic spheres and were stored in −80° C. freezer until analysis. The tissues were placed on ice under laminar flow and 750 μL Trizol (ThermoFisher 15596026/15596018) was immediately added to each tube. The tissue was disrupted and homogenized using the FastPrep-24™ 5G Grinder with 3 cycles of 30 sec at speed 5 m/sec. Between cycles, the samples were cooled down on ice for 2 minutes. After homogenization, the tube was shortly spun and 20% volume of Chloroform (150 μL Chloroform when 750 μL Trizol was used) was added. The tube was vortexed for 15 sec and centrifuged at 14.000×g for 15 min at 4° C. The upper aqueous phase was transferred to a deep 96-well plate, and 1 volume of 70% ethanol was added and mixed well.

The next steps were performed using RNeasy 96 kit (Qiagen) following manufacture's protocol. An RNeasy 96 plate was placed on top of the Square-Well Block holder, the samples (Trizol/Chloroform extraction) were applied into the wells of the RNeasy 96 plate, and the plate was sealed with AirPore cover to prevent contamination. The samples were centrifuged at 5600×g for 3 min at RT and the solution was discarded. The samples were serially washed in steps including 1 time of 800 μL RW1 buffer, 2 times of 800 μL RPE buffer that were applied to the RNeasy 96 plate; wash buffers were removed by centrifugation at 5600×g for 3 min for each step. After the last wash, the RNeasy 96 plate was centrifuged at 5600×g for 3 min to remove the residual lipid. RNA was eluted using 60 μL RNase-free water by centrifugation at 5600×g for 3 min at RT. RNA concentration was measured by Nanodrop 8000 (ThermoFisher). The eluted RNA was stored at −80° C. until analysis.

### RT-qPCR

RNA was used as template for reverse transcription using and was reversed transcribed using High-Capacity cDNA Reverse Transcription Kits (Applied Biosystems) following manufacture's protocol. Briefly, a final reaction of 20 μL mixture was incubated at 25° C. for 10 min, followed by reverse transcription at 37° C. for 2 hours and enzyme inactivation at 85° C. for 5 min. For qPCR reaction, reverse transcribed cDNAs were diluted 10 times and mixed with 2× PowerUp™ SYBR™ Green Mater Mix (ThermoFisher A25743) and 500 nM qPCR primers to a final volume of 10 μL reaction. The qPCR runs were performed with a QuantStudio™ 12K instrument (Applied Biosystems™) using standard thermal cycling protocol. Multiple primers were used to detect MAPT mRNA, and reference primers targeting mouse housekeeping genes AP3D1 and PAKIIP1 were included for normalization of gene expression. The primer sequences are listed in Table 8. qPCR data were analysed using qBase+ software (Biogazelle).

Protein Extraction from Tissues and MSD Immunoassay

Tissues were collected in Lysing Matrix D (MP Biomedicals 6913-500) 2 mL tubes containing 1.4 mm ceramic spheres and stored in −80° C. freezer until analysis. Samples were transferred on ice and kept on ice, 6- to 20-volume (μl)/weight (mg) of ice-cold RIPA buffer (Sigma) supplemented with cOmplete™ Protease inhibitor cocktail (Roche) and PhosSTOP™ (Roche) were added to cortex and hippocampus tissues, and the samples were left thawing on ice for 15 minutes. Samples were homogenized in FastPrep-24 instrument (MP Biomedicals) with 6.0 m/s for 20 s (1 cycle). The total homogenate was centrifuged at 18,000 g for 40 min in Eppendorf centrifuge at 4° C.

The tissue lysates were collected and diluted 2500 folds for protein measurement using MSD immunoassays following a standardized procedure. Briefly, MSD plates were coated with 30 μL per well of coating antibody diluted in PBS at 1 μg/mL overnight at 4° C. The next day, plates were inverted on absorbent tissue to dry and afterwards incubated with 150 μL per well blocking buffer 0.1% Casein for 2 hours at RT with orbital shaking at 300 rpm. After blocking, the plates were incubated with cell lysate diluted in RIPA buffer supplemented with cOmplete™ Protease inhibitor cocktail (Roche) overnight with slow orbital shaking at 4° C. Plates were washed 5× times using Titertek Aquamax 4000 and inverted on absorbent tissue to dry. Afterwards, 25 μL/per secondary or detection antibody diluted in 0.1% casein were added to MSD plates and incubated for 2 hours at room temperature. After antibody incubation, plates were washed 5× times and developed with 150 μL/well 2×MSD reading buffer (Meso Scale Discovery) diluted in milliQ H2O. Plates were read immediately using MSD instrument. For measurement of Tau protein, hTau43 antibody (Janssen) was used for 96-well MSD plate coating, and SULFO-TAG™ labeled hTau60 antibody (Janssen) was used for detection.

### 7.7 Example 7: In Vitro Knockdown Efficiency Assessment of siRNA Conjugates in Human iPSC-Derived Neurons

The in vitro knockdown efficiencies of MAPT lipid-siRNA conjugates were assessed in human iPSC-derived cortical neurons following the procedure described below.

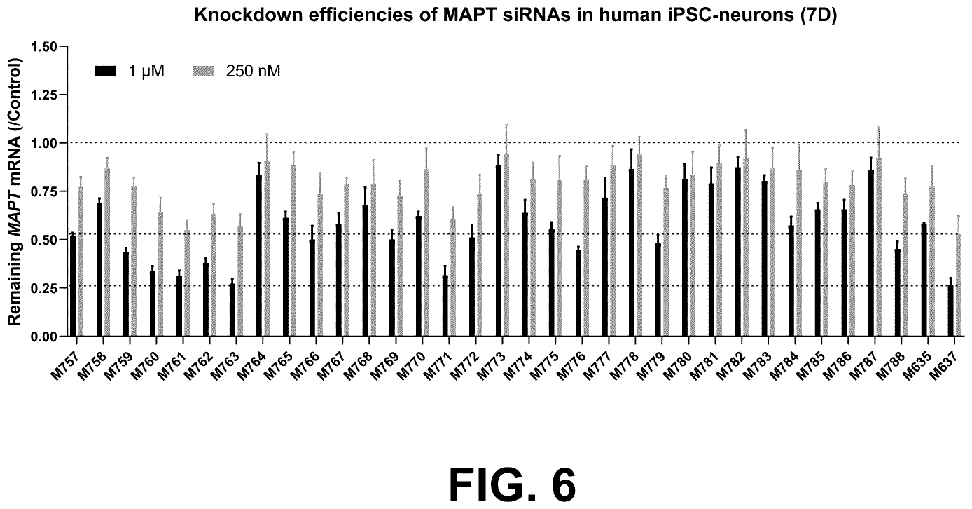

FIG. 6 and Table 34 summarize the efficiency of the MAPT lipid-siRNA conjugates in knocking down MAPT mRNA in human iPSC-neurons after incubation for 7 days. MAPT lipid-siRNA conjugates M757-MM788 are described in Table 33, and M635 (also known as M165_Var5) and M637 (also known as M177_Var1) are described in Table 5. Data are presented in Table 34 as remaining MAPT mRNA relative to control. Mean±S.D., n=3.

Differentiation of Human iPSCs into Cortical Neurons

The human iPSC line (Sigma #iPSC0028) was derived with OSKM retroviral reprogramming of epithelial cells from a 24-years old Caucasian female donor. iPSCs were first differentiated into cortical neural stem cells (NSCs) following a dual SMAD inhibitor protocol (Shi et al., 2012, Nat. Proc. 7(10):1836-46) with some modifications. Briefly, iPSCs were plated at 500,000 cells/cm² on wells coated with Matrigel (Corning 354230) in mTeSR medium (StemCell Technologies 5850) supplemented with 10 μM ROCK inhibitor (Sigma-Aldrich Y0503) and cultured at 37° C. and with 5% 02. The media were replaced with mTeSR the next day (day −1).

From day 0 until day 12, cell media were changed every day with cortical Neural Induction Medium comprised of 10 μM SB43142 (Tocris 1614) and 1 μM Dorsomorphin (Tocris 3093) supplemented in Neural Maintenance Medium (1:1 DMEM:F12 Glutamax (ThermoFisher Scientific 10565108), Neurobasal (ThermoFisher Scientific 21103049), 2.5 μg/mL Insulin (Sigma-Aldrich I9278-5 ML), 50 uM 2-mercaptoethanol (ThermoFisher Scientific, 31350010), 0.5% Non-Essential Amino Acids (ThermoFisher Scientific 11140035), 0.5% GlutaMAX supplement (ThermoFisher Scientific, 10565018), 0.5 mM Sodium Pyruvate (ThermoFisher Scientific 11360070), 1% Penicillin-Streptomycin (Sigma P4333), 0.5% N₂ supplement (ThermoFisher Scientific 17502048), 1% B27 supplement (ThermoFisher Scientific 17504044). At day 12, the neuroepithelial sheet was gently detached into large aggregates of 300 to 500 cells using a needle and lifter and the clumps were collected in a 15 mL falcon by centrifugation at 160 g for 2 min. Cell pellets were gently resuspended in Neural Induction Medium for a ½ or ⅓ passage into wells coated with 10 μg/mL laminin (Sigma-Aldrich L2020) in a total volume of 2 mL of Neural Induction Medium per well of the 6-well plate.

Media were changed at day 13 and day 15 into Neural Maintenance Medium supplemented with 20 ng/mL of FGF2 (Stemcell Technologies 2634). At day 17, the neural rosettes were detached with dispase (ThermoFisher Scientific 17105041) for a ⅓ passage and plated into laminin-coated wells. For another week, 1 or 2 extra dispase steps were performed.

At around days 25-30, neural stem cells were dissociated with Accutase (ThermoFisher Scientific A1110501) into single-cell suspension and cryopreserved in freshly prepared Neural Freezing Medium containing Neural Maintenance Medium supplemented with 10% (V/V) DMSO and 20 ng/mL FGF2. The frozen vials of neural stem cells were stored in liquid nitrogen until use.

To generate iPSC-neurons, frozen vials of neural stem cells (NSCs) were thawed and plated on laminin-coated wells at 70,000 cells/cm² in Neural Maintenance Medium supplemented with 10 μM ROCK inhibitor and 20 ng/mL FGF2. In the following two days, media were replaced daily with neural maintenance medium. At around day 4 after thawing, cells were dissociated with Accutase and placed at 28.000 cells per well in 96-well plates pre-coated with poly-L-ornithine and laminin in Neural Maintenance Medium supplemented with 10 μM ROCK inhibitor. The next day after re-plating, culture media were replaced with Neural Differentiation Medium comprised of Neural Maintenance Medium supplemented with 20 ng/mL BDNF (R & D Systems 212-BD-050/CF), 20 ng/mL GDNF (R & D Systems 212-GD-050/CF), 500 μM DB-cAMP (Sigma D0627) and 20 mM Ascorbic Acid (Sigma A4403). Cultures were differentiated in Neural Differentiation Medium with 50% medium change twice per week. Two-to-three weeks after differentiation from neural stem cells (NSCs), neurons were treated with siRNA for 7 days or 14 days for RNA analysis by Reverse transcription and Real time PCR.

TABLE 27
Remaining human MAPT mRNA (% vehicle control) in cortex
from hTAU KI mice after injection with M637

| | Vehicle | | M637 (30 mole) | | M637 (5 mole) | | M637 (1 mole) | |
|---|---|---|---|---|---|---|---|---|
| Day | Mean | SD | Mean | SD | Mean | SD | Mean | SD |
| 7 | 100.0 | 2.3 | 22.9 | 6.4 | 52.1 | 11.7 | 83.1 | 2.4 |
| 28 | 100.0 | 3.4 | 15.8 | 1.4 | 41.7 | 6.0 | 73.6 | 6.9 |
| 56 | 100.0 | 3.4 | 18.8 | 4.8 | 49.8 | 6.9 | 72.2 | 6.1 |
| 84 | 100.0 | 7.8 | 15.1 | 1.4 | 47.3 | 6.5 | 76.3 | 5.1 |
| 112 | 100.0 | 4.9 | 21.3 | 3.1 | 52.7 | 12.0 | 86.7 | 8.7 |
| 147 | 100.0 | 2.2 | 28.6 | 7.4 | 65.0 | 8.4 | 88.1 | 4.0 |

TABLE 28
Remaining human MAPT mRNA (% vehicle control) in hippocampus
from hTAU KI mice after injection with M637

| | Vehicle | | M637 (30 mole) | | M637 (5 mole) | | M637 (1 mole) | |
|---|---|---|---|---|---|---|---|---|
| Day | Mean | SD | Mean | SD | Mean | SD | Mean | SD |
| 7 | 100.0 | 2.4 | 16.7 | 5.1 | 34.0 | 6.9 | 67.0 | 2.6 |
| 28 | 100.0 | 3.9 | 10.6 | 3.0 | 25.8 | 4.9 | 47.4 | 11.6 |
| 56 | 100.0 | 6.4 | 14.0 | 2.4 | 31.6 | 4.0 | 50.0 | 7.8 |
| 84 | 100.0 | 3.6 | 10.8 | 2.4 | 31.0 | 6.2 | 50.7 | 8.6 |
| 112 | 100.0 | 5.3 | 15.2 | 2.7 | 35.8 | 9.4 | 71.5 | 5.6 |
| 147 | 100.0 | 0.9 | 19.3 | 4.8 | 48.5 | 12.9 | 76.6 | 6.3 |

TABLE 29
Remaining human Tau protein (% vehicle control) in
cortex from hTAU KI mice after injection with M637

| | Vehicle | | M637 (30 mole) | | M637 (5 mole) | | M637 (1 mole) | |
|---|---|---|---|---|---|---|---|---|
| Day | Mean | SD | Mean | SD | Mean | SD | Mean | SD |
| 7 | 100.0 | 20.4 | 68.6 | 18.1 | 89.2 | 15.6 | 98.6 | 9.1 |
| 28 | 100.0 | 13.0 | 27.2 | 3.8 | 49.5 | 3.1 | 77.2 | 8.4 |
| 56 | 100.0 | 12.1 | 14.9 | 4.1 | 39.8 | 10.1 | 61.5 | 13.5 |
| 84 | 100.0 | 15.7 | 6.4 | 1.7 | 35.2 | 7.7 | 70.9 | 9.1 |
| 112 | 100.0 | 13.0 | 6.6 | 3.2 | 31.0 | 4.7 | 72.9 | 13.9 |
| 147 | 100.0 | 5.3 | 15.7 | 6.1 | 58.5 | 8.5 | 86.8 | 9.2 |

TABLE 30
Remaining human Tau protein (% vehicle control) in hippocampus
from hTAU KI mice after injection with M637

| | Vehicle | | M637 (30 mole) | | M637 (5 mole) | | M637 (1 mole) | |
|---|---|---|---|---|---|---|---|---|
| Day | Mean | SD | Mean | SD | Mean | SD | Mean | SD |
| 7 | 100.0 | 16.0 | 61.5 | 4.3 | 79.0 | 11.1 | 92.9 | 11.0 |
| 28 | 100.0 | 12.6 | 21.9 | 3.7 | 36.7 | 6.7 | 60.7 | 9.3 |
| 56 | 100.0 | 13.9 | 11.9 | 3.5 | 27.5 | 6.2 | 49.1 | 15.9 |
| 84 | 100.0 | 23.3 | 3.5 | 1.9 | 21.7 | 6.1 | 46.1 | 10.5 |
| 112 | 100.0 | 17.1 | 6.0 | 2.3 | 30.1 | 7.0 | 66.2 | 7.0 |
| 147 | 100.0 | 2.1 | 9.0 | 6.2 | 42.5 | 9.0 | 78.0 | 14.3 |

TABLE 31
Remaining human MAPT mRNA (% vehicle control) in cortex from
hTAU KI mice after injection with M635, M639, or M641

| | Vehicle | | M635 (30 mole) | | M639 (30 mole) | | M641 (30 mole) | |
|---|---|---|---|---|---|---|---|---|
| Day | Mean | SD | Mean | SD | Mean | SD | Mean | SD |
| 28 | 100.0 | 4.6 | 21.2 | 8.2 | 12.9 | 2.2 | 17.3 | 4.7 |
| 84 | 100.0 | 6.4 | 35.7 | 12.5 | 16.1 | 2.7 | 22.4 | 5.3 |

TABLE 32
Remaining human MAPT mRNA (% vehicle control) in hippocampus
from hTAU KI mice after injection with M635, M639, or M641

| | Vehicle | | M635 (30 mole) | | M639 (30 mole) | | M641 (30 mole) | |
|---|---|---|---|---|---|---|---|---|
| Day | Mean | SD | Mean | SD | Mean | Mean | SD | Mean |
| 28 | 100.0 | 3.5 | 17.3 | 6.1 | 9.6 | 2.2 | 12.5 | 3.8 |
| 64 | 100.0 | 2.3 | 20.9 | 6.7 | 11.8 | 1.7 | 16.9 | 3.9 |

TABLE 34
Remaining MAPT mRNA (% vehicle control) in
human iPSC-derived cortical neurons after
incubation with MAPT lipid-siRNA conjugates

| | | |
|---|---|---|
| MAPT lipid- | 1 μM siRNA | 250 nM siRNA |

| siRNA conjugate | Mean | SD | Mean | SD |
|---|---|---|---|---|
| M757 | 0.521 | 0.015 | 0.772 | 0.053 |
| M758 | 0.687 | 0.027 | 0.868 | 0.055 |
| M759 | 0.438 | 0.015 | 0.774 | 0.043 |
| M760 | 0.337 | 0.026 | 0.641 | 0.076 |
| M761 | 0.313 | 0.027 | 0.550 | 0.048 |
| M762 | 0.379 | 0.023 | 0.632 | 0.056 |
| M763 | 0.273 | 0.023 | 0.567 | 0.064 |
| M764 | 0.836 | 0.061 | 0.905 | 0.139 |
| M765 | 0.612 | 0.033 | 0.884 | 0.069 |
| M766 | 0.501 | 0.070 | 0.737 | 0.105 |
| M767 | 0.582 | 0.055 | 0.786 | 0.035 |
| M768 | 0.679 | 0.091 | 0.789 | 0.122 |
| M769 | 0.502 | 0.049 | 0.730 | 0.074 |
| M770 | 0.622 | 0.023 | 0.864 | 0.108 |
| M771 | 0.316 | 0.048 | 0.604 | 0.063 |
| M772 | 0.512 | 0.065 | 0.736 | 0.097 |
| M773 | 0.884 | 0.055 | 0.947 | 0.146 |
| M774 | 0.639 | 0.066 | 0.810 | 0.089 |
| M775 | 0.553 | 0.037 | 0.807 | 0.126 |
| M776 | 0.445 | 0.018 | 0.808 | 0.073 |
| M777 | 0.717 | 0.102 | 0.883 | 0.102 |
| M778 | 0.865 | 0.102 | 0.941 | 0.090 |
| M779 | 0.481 | 0.042 | 0.768 | 0.063 |
| M780 | 0.810 | 0.078 | 0.833 | 0.120 |
| M781 | 0.790 | 0.082 | 0.898 | 0.089 |
| M782 | 0.873 | 0.053 | 0.923 | 0.145 |
| M783 | 0.803 | 0.030 | 0.872 | 0.102 |
| M784 | 0.574 | 0.045 | 0.860 | 0.129 |
| M785 | 0.656 | 0.033 | 0.796 | 0.071 |
| M786 | 0.656 | 0.049 | 0.781 | 0.074 |
| M787 | 0.858 | 0.066 | 0.921 | 0.159 |
| M788 | 0.451 | 0.039 | 0.740 | 0.081 |
| M635 | 0.582 | 0.004 | 0.774 | 0.105 |
| M637 | 0.264 | 0.038 | 0.528 | 0.094 |

The invention is not to be limited in scope by the specific embodiments described herein. Indeed, various modifications of the invention in addition to those described will become apparent to those skilled in the art from the foregoing description and accompanying Figures. Such modifications are intended to fall within the scope of the appended claims.

All references cited herein are incorporated herein by reference in their entirety and for all purposes to the same extent as if each individual publication or patent or patent application was specifically and individually indicated to be incorporated by reference in its entirety for all purposes.

1.-27. (canceled)

28. An siRNA comprising a sense strand and an antisense strand forming a double stranded duplex, wherein
(a) the sense strand comprises 21 nucleotides having the sequence: CAAGUCCAAGAUCGGCUCCAA, wherein from the 5′end
(i) the nucleotide at one or more of positions 1-6, 8, and 12-21 is optionally modified with 2′-O-methyl;
(ii) the nucleotide at one or more of positions 9-11 is optionally modified with 2′-deoxy-2′fluoro;
(iii) the nucleotide at position 7 optionally comprises a lipophilic moiety;
wherein the sense strand optionally further comprises an inverse abasic nucleotide (InvAb) at the 5′ end linked to the nucleotide at position 1 and/or an inverse abasic nucleotide (InvAb) at the 3′ end linked to the nucleotide at position 21, and wherein the sense strand optionally comprises at least two phosphorothioate linkages at the 5′end and/or at least two phosphorothioate linkages at the 3′end; and
(b) the antisense strand comprises 21 nucleotides having the sequence: UUGGAGCCGAUCUUGGACUUG, wherein from the 5′end
(i) the nucleotide at position 1 is optionally modified with vinyl phosphonate 2′-O-methyl or cis-cyclobutyl phosphonate 2′-O-methyl;
(ii) the nucleotide at one or more of positions 2, 14, and 16 is optionally modified with 2′-deoxy-2′fluoro;
(iii) the nucleotide at one or more of positions 1, 3-6, 8-13, 15, and 17-21 is optionally modified with 2′-O-methyl;
(iv) optionally, the nucleotide at one or more of positions 4, 5, and 7 is a deoxynucleotide;
(v) optionally, the nucleotide at position 7 is a 3′-O-methyl modified nucleotide with 2′-5′ linked phosphate, a (L)-α-threofuranosyl (3′-2′) modified nucleotide, or a glycol nucleic acid (GNA), or an unlocked nucleic acid (UNA);
wherein the antisense strand optionally comprises at least two phosphorothioate linkages at the 5′end and/or at least two phosphorothioate linkages at the 3′end; and
wherein the base pair at position 1 of the 5′-end of the antisense strand of the duplex is an AU base pair.

29. The siRNA of claim 28, wherein in the sense strand, the nucleotide at two or more of positions 1-6, 8 and 12-21 is modified with 2′-O-methyl.

30. The siRNA of claim 28, wherein in the sense strand, the nucleotide at three or more of positions 1-6, 8 and 12-21 is modified with 2′-O-methyl.

31. The siRNA of claim 28 wherein in the sense strand, the nucleotide at five or more of positions 1-6, 8 and 12-21 is modified with 2′-O-methyl.

32. The siRNA of claim 28, wherein in the sense strand, the nucleotide at each of positions 1-6, 8, and 12-21 is modified with 2′-O-methyl.

33. The siRNA of claim 28, wherein in the sense strand, the nucleotide at two or more of positions 9-11 is modified with 2′-deoxy-2′fluoro.

34. The siRNA of claim 28, wherein in the sense strand, the nucleotide at each of positions 9-11 is modified with 2′-deoxy-2′fluoro.

35. The siRNA of claim 28, wherein in the sense strand, the nucleotide at position 7 comprises lipophilic moiety L22.

36. The siRNA of claim 28, wherein the sense strand further comprises an InvAb at both the 5′ end linked to the nucleotide at position 1 and at the 3′ end linked to the nucleotide at position 21.

37. The siRNA of claim 28, wherein the sense strand comprises two phosphorothioate linkages at both the 5′end and the 3′end.

38. The siRNA of claim 28, wherein in the antisense strand, the nucleotide at two or more of positions 1, 3-6, 8-13, 15, and 17-21 is modified with 2′-O-methyl.

39. The siRNA of claim 28, wherein in the antisense strand, the nucleotide at three or more of positions 1, 3-6, 8-13, 15, and 17-21 is modified with 2′-O-methyl.

40. The siRNA of claim 28, wherein in the antisense strand, the nucleotide at five or more of positions 1, 3-6, 8-13, 15, and 17-21 is modified with 2′-O-methyl.

41. The siRNA of claim 28, wherein in the antisense strand, the nucleotide at each of positions 1, 3, 6, 8-13, 15, and 17-21 is modified with 2′-O-methyl.

42. The siRNA of claim 28, wherein in the antisense strand, the nucleotide at two or more of positions 2, 14, and 16 is modified with 2′-deoxy-2′fluoro.

43. The siRNA of claim 28, wherein in the antisense strand, wherein in the antisense strand, the nucleotide at each of positions 2, 14, and 16 is modified with 2′-deoxy-2′fluoro.

44. The siRNA of claim 28, wherein in the antisense strand, the nucleotides at position 4 and at position 5 are each modified with 2′-O-methyl.

45. The siRNA of claim 28, wherein in the antisense strand, the nucleotide at position 4 is modified with 2′-O-methyl and the nucleotide at position 5 is a deoxynucleotide.

46. The siRNA of claim 28, wherein in the antisense strand, the nucleotide at position 4 is a deoxynucleotide and the nucleotide at position 5 is modified with 2′-O-methyl.

47. The siRNA of claim 28, wherein the antisense strand comprises two phosphorothioate linkages at both the 5′end and the 3′end.

48. The siRNA of claim 28, wherein the sense strand comprises nucleotides having the sequence of SEQ ID NO: 333, 541, 547, 557, or 569.

49. The siRNA of claim 28, wherein the antisense strand comprises nucleotides having the sequence of SEQ ID NO: 334, 542, 548, 558, or 570.

50. An siRNA comprising a sense strand and an antisense strand forming a double stranded duplex, wherein the sense strand comprises nucleotides having the sequence of SEQ ID NO: 333, and the antisense strand comprises nucleotides having the sequence of SEQ ID NO: 334.

51.-62. (canceled)

63. An siRNA comprising a sense strand and an antisense strand forming a double stranded duplex, wherein the sense strand comprises nucleotides having the sequence of SEQ ID NO: 541, and the antisense strand comprises nucleotides having the sequence of SEQ ID NO: 542.

64. An siRNA comprising a sense strand and an antisense strand forming a double stranded duplex, wherein the sense strand comprises nucleotides having the sequence of SEQ ID NO: 547, and the antisense strand comprises nucleotides having the sequence of SEQ ID NO: 548.

65. An siRNA comprising a sense strand and an antisense strand forming a double stranded duplex, wherein the sense strand comprises nucleotides having the sequence of SEQ ID NO: 557, and the antisense strand comprises nucleotides having the sequence of SEQ ID NO: 558.

66. An siRNA comprising a sense strand and an antisense strand forming a double stranded duplex, wherein the sense strand comprises nucleotides having the sequence of SEQ ID NO: 567, and the antisense strand comprises nucleotides having the sequence of SEQ ID NO: 568.

67. An siRNA comprising a sense strand and an antisense strand forming a double stranded duplex, wherein the sense strand comprises nucleotides having the sequence of SEQ ID NO: 569, and the antisense strand comprises nucleotides having the sequence of SEQ ID NO: 570.

68. A composition comprising the siRNA agent of claim 28, and a carrier.

69. A method of inhibiting expression of MAPT gene in a cell or population of cells, the method comprising contacting the cell or population of cells with the siRNA of claim 28.

70.-75. (canceled)
